(19) United States
(12) Patent Application Publication
(10) Pub. No.: US 2025/0311949 A1
(43) Pub. Date: Oct. 9, 2025

(54) WEARABLE DEVICE WITH PHYSIOLOGICAL PARAMETERS MONITORING

(71) Applicant: MASIMO CORPORATION, Irvine, CA (US)

(72) Inventors: Ammar Al-Ali, San Juan Capistrano, CA (US); Stephen Scruggs, Newport Beach, CA (US); Richard Priddell, Irvine, CA (US); Walter M. Weber, Laguna Hills, CA (US); Sung Uk Lee, Irvine, CA (US); Anmol B. Majmudar, Irvine, CA (US); Sean Devlin, Irvine, CA (US)

(21) Appl. No.: 19/079,292

(22) Filed: Mar. 13, 2025

Related U.S. Application Data
(60) Provisional application No. 63/565,146, filed on Mar. 14, 2024.
(60) Provisional application No. 63/570,691, filed on Mar. 27, 2024.
(60) Provisional application No. 63/571,738, filed on Mar. 29, 2024.

(51) Int. Cl. A61B 5/1455; A61B 5/00

(52) U.S. Cl. CPC A61B 5/14552; A61B 5/681

(57) ABSTRACT

Various wearable health monitoring devices are described herein. Some implementations include a housing including a front side and a back side configured to face tissue of the wearer when the device is worn; and a sensor assembly positioned by the back side of the housing. The sensor assembly can include emitters and detectors positioned on a surface of a substrate and a frame positioned adjacent to the surface of the substrate and configured to inhibit transmission of optical radiation through the frame. The frame can comprise an emitter chamber housing the emitters; and one or more detector chambers housing the one or more detectors.

### INCORPORATION BY REFERENCE TO ANY PRIORITY APPLICATIONS

The present application claims priority to U.S. Provisional Application No. 63/565,146, filed Mar. 14, 2024, U.S. Provisional Application No. 63/570,691, filed Mar. 27, 2024, and U.S. Provisional Application No. 63/571,738, filed Mar. 29, 2024. All of the above-listed applications, and any and all applications for which a foreign or domestic priority claim is identified in the Application Data Sheet as filed with the present application, are hereby incorporated by reference under 37 CFR 1.57.

### TECHNICAL FIELD

The present disclosure relates to a wearable health monitoring device incorporating a plurality of sensors worn on the wrist.

### BACKGROUND

Spectroscopy is a common technique for measuring the concentration of organic and some inorganic constituents of a solution. The theoretical basis of this technique is the Beer-Lambert law, which states that the concentration cᵢ of an absorbent in solution can be determined by the intensity of light transmitted through the solution, knowing the pathlength d_{λ}, the intensity of the incident light I_{0,λ}, and the extinction coefficient ε_{i,λ} at a particular wavelength λ.

In generalized form, the Beer-Lambert law is expressed as the following equations, where μα,λ is the bulk absorption coefficient and represents the probability of absorption per unit length:

I_{λ}=I_{0,λ}e^{-d}^{_{λ}}^{·μ}^{_{α,λ}} (1)

μ_{α,λ}=Σᵢ₌₁ⁿε_{i,λ}·cᵢ (2)

The minimum number of discrete wavelengths that are required to solve equations 1 and 2 is the number of significant absorbers that are present in the solution.

A practical application of this technique is pulse oximetry or plethysmography, which utilizes a noninvasive sensor to measure oxygen saturation and pulse rate, among other physiological parameters. Pulse oximetry or plethysmography relies on a sensor attached externally to the patient (typically for example, at the fingertip, foot, ear, forehead, or other measurement sites) to output signals indicative of various physiological parameters, such as a patient's blood constituents and/or analytes, including for example a percent value for arterial oxygen saturation, among other physiological parameters. The sensor has at least one emitter that transmits optical radiation of one or more wavelengths into a tissue site and at least one detector that responds to the intensity of the optical radiation (which can be reflected from or transmitted through the tissue site) after absorption by pulsatile arterial blood flowing within the tissue site. Based upon this response, a processor determines the relative concentrations of oxygenated hemoglobin (HbO₂) and deoxygenated hemoglobin (Hb) in the blood so as to derive oxygen saturation, which can provide early detection of potentially hazardous decreases in a patient's oxygen supply, and other physiological parameters.

A patient monitoring device can include a plethysmograph sensor. The plethysmograph sensor can calculate oxygen saturation (SpO₂), pulse rate, a plethysmograph waveform, perfusion index (PI), pleth variability index (PVI), methemoglobin (MetHb), carboxyhemoglobin (CoHb), total hemoglobin (tHb), respiration rate, glucose, and/or otherwise. The parameters measured by the plethysmograph sensor can display on one or more monitors the foregoing parameters individually, in groups, in trends, as combinations, or as an overall wellness or other index.

A pulse oximetry sensor is described in U.S. Pat. No. 6,088,607 entitled Low Noise Optical Probe; pulse oximetry signal processing is described in U.S. Pat. Nos. 6,650,917 and 6,699,194 entitled Signal Processing Apparatus and Signal Processing Apparatus and Method, respectively; a pulse oximeter monitor is described in U.S. Pat. No. 6,584,336 entitled Universal/Upgrading Pulse Oximeter; all of which are assigned to Masimo Corporation, Irvine, CA, and each is incorporated by reference herein in its entirety.

### SUMMARY

A drawback to current pulse oximetry sensors is a need to be located near significant capillary beds on the body, including fingers, ears, toes, nose and forehead. Such locations are often inconvenient for monitoring a user during normal activities, outside of a healthcare facility. Further, although measuring through motion oxygen saturation technology exists, it is directed to the healthcare facility context and is not reliable for normal routines, which include sporting activities or other significant daily movement. Accordingly, the present disclosure provides a sensor which allows for measuring pulse oximetry at sparse capillary bed locations, including the wrist. The present disclosure also provides algorithms for measuring pulse oximetry though higher exertion everyday motion.

It is noted that “plethysmograph” as used herein (commonly referred to as “photoplethysmograph”), encompasses its broad ordinary meaning known to one of skill in the art, which includes at least data representative of a change in the absorption of particular wavelengths of light as a function of the changes in body tissue resulting from pulsing blood. Moreover, “oximetry” as used herein encompasses its broad ordinary meaning known to one of skill in the art, which includes at least those noninvasive procedures for measuring parameters of circulating blood through spectroscopy.

For purposes of summarization, certain aspects, advantages and novel features are described herein. Of course, it is to be understood that not necessarily all such aspects, advantages or features need to be present in any particular aspect.

Disclosed herein is a wearable health monitoring device configured to be secured to a wrist of a user, the wearable health monitoring device including: a circuit board; a first emitter mounted to a surface of the circuit board and configured to emit optical radiation towards tissue of the user's wrist; a second emitter mounted to said surface of the circuit board and configured to emit optical radiation towards said tissue; a plurality of detectors mounted to said surface of the circuit board and spaced from one another, said plurality of detectors configured to detect optical radiation emitted from the first and second emitters after attenuation by said tissue and further configured to output one or more signals based on said detected optical radiation; a frame positioned adjacent to said surface of the circuit board, the frame including glass infused with optically absorbent pigment, the frame including a plurality of walls forming: a first emitter chamber; a second emitter chamber; and a plurality of detector chambers spaced from one another and surrounding both of the first and second emitter chambers; wherein said first emitter is arranged within the first emitter chamber and said second emitter is arranged within the second emitter chamber, wherein said plurality of detectors are arranged within said plurality of detector chambers, and wherein the plurality of walls of the frame are configured to: (i) inhibit optical radiation emitted from the first emitter from entering the second emitter chamber; (ii) inhibit optical radiation emitted from the second emitter from entering the first emitter chamber; and (iii) inhibit optical radiation emitted from the first and second emitters from entering the plurality of detector chambers without first reaching at least a portion of said tissue; a first emitter chamber cover covering an opening of the first emitter chamber, the first emitter chamber cover including optically transmissive glass; a second emitter chamber cover covering an opening of the second emitter chamber, the second emitter chamber cover including optically transmissive glass; and a plurality of detector chamber covers, each of said plurality of detector chamber covers covering an opening of a different one of said plurality of detector chambers, wherein the first emitter chamber, the second emitter chamber, the plurality of detector chamber covers, and the frame include a single unitary structure.

In some implementations, the first emitter chamber, the second emitter chamber, the plurality of detector chamber covers, and the frame are fused together as a single contiguous mass.

In some implementations, the optically absorbent pigment is distributed throughout the plurality of walls of the frame rendering the plurality of walls optically opaque.

In some implementations, the frame is overmolded over the first and second emitter chamber covers and the plurality of detector chamber covers.

In some implementations, the optically absorbent pigment is black.

In some implementations, the first emitter chamber cover and the second emitter chamber cover are integrally formed from the same material.

In some implementations, the first emitter chamber cover and the second emitter chamber cover are connected to one another by one or more bridging portions.

In some implementations, the first emitter chamber cover, the second emitter chamber cover, and the one or more bridging portions are integrally formed from the same material.

In some implementations the first emitter chamber cover includes: a first portion sized and shaped to correspond to a size and shape of the opening of the first emitter chamber; and a second portion connected to the first portion and embedded with the frame. In some implementations: the second emitter chamber cover includes: a first portion sized and shaped to correspond to a size and shape of the opening of the second emitter chamber; and a second portion connected to the first portion of the second emitter chamber cover and embedded with the frame.

In some implementations, the first portion of the first emitter chamber cover protrudes from the second portion of the first emitter chamber cover; and the first portion of the second emitter chamber cover protrudes from the second portion of the second emitter chamber cover.

In some implementations, the second portion of the first emitter chamber cover extends around a perimeter of the first portion of the first emitter chamber; and the second portion of the second emitter chamber cover extends around a perimeter of the first portion of the second emitter chamber.

In some implementations, the second portion of the first emitter chamber and the second portion of the second emitter chamber cover are connected to one another by one or more bridging portions.

In some implementations, said one or more bridging portions includes two bridging portions separated from one another.

In some implementations, the first and second portions of the first emitter chamber, the first and second portions of the second emitter chamber, and the one or more bridging portions are integrally formed from the same material.

In some implementations, the plurality of detector chamber covers are integrally formed from the same material.

In some implementations, the plurality of detector chamber covers are connected to one another.

In some implementations, each of the plurality of detector chamber covers include: a first portion sized and shaped to correspond to a size and shape of respective openings of the plurality of detector chambers; and a second portion connected to the first portion and embedded with the frame.

In some implementations, respective first portions of each detector chamber cover protrudes from respective second portions of each detector chamber cover.

In some implementations, respective second portions of each detector chamber cover extend around a perimeter of respective first portions of each detector chamber cover.

In some implementations, the first and second portions of the plurality of detector chamber covers are integrally formed from the same material.

In some implementations, the second portions of the plurality of detector chamber covers are connected to one another.

In some implementations: the plurality of detector chamber covers includes a skirt wall that is connected to the second portions of the plurality of detector chamber covers; the skirt wall is centrally disposed within the plurality of detector chamber covers; the skirt wall forms a closed loop; and/or the skirt wall extends from the second portions of the plurality of detector chamber covers.

In some implementations: each of said plurality of walls of the frame includes a first end that is adjacent to said surface of the circuit board and a second end opposite said first end; and the first emitter chamber cover, the second chamber emitter chamber cover, the plurality of detector chamber covers, and the second ends of the plurality of walls of the frame form a continuous, curved surface of the wearable device that is configured to contact the user's tissue when the wearable device is secured to the user.

In some implementations, the wearable health monitoring device further includes: a housing configured to be connected to at least one strap for securing the housing to the user's wrist, the housing including a top portion, a bottom portion configured to face towards the user's wrist, and an opening in the bottom portion; and a sensor assembly configured to be at least partially retained by the opening of the housing, wherein the sensor assembly includes the circuit board, the first emitter, the second emitter, the plurality of detectors, and the frame.

In some implementations, the first emitter includes a plurality of LEDs configured to emit optical radiation of at least three wavelengths and/or the second emitter includes a plurality of LEDs configured to emit optical radiation of at least three wavelengths.

Disclosed herein is a wearable health monitoring device including: one or more emitter chamber covers injection molded out of a first material; one or more detector chamber covers injection molded out of a second material; and a light barrier construct injection molded out of a third material over the one or more emitter chamber covers and the one or more detector chamber covers; wherein: the first material includes glass and is optically transmissive; the second material includes glass and is optically transmissive; and the third material includes glass and an optically absorbent pigment.

In some implementations, the first material is transparent, wherein the second material is transparent, wherein the third material is opaque.

In some implementations, the optically absorbent pigment is black.

In some implementations, the third material is colored with the optically absorbent pigment.

In some implementations, the optically absorbent pigment is distributed throughout the light barrier construct.

In some implementations, the glass of the light barrier construct is infused with the optically absorbent pigment.

In some implementations, the first and second materials do not include the optically absorbent pigment.

In some implementations, the first material of the one or more emitter chamber covers is less transparent than the second material of the one or more detector chamber covers to allow the one or more emitter chamber covers to diffuse optical radiation passing therethrough.

In some implementations, the second material of the one or more detector chamber covers includes texture configured to diffuse optical radiation passing through the second material.

In some implementations, the light barrier construct is overmolded over the one or more emitter chamber covers and the one or more detector chamber covers.

In some implementations, the light barrier construct, the one or more emitter chamber covers, and the one or more detector chamber covers are integrally formed as a contiguous mass.

In some implementations, the light barrier construct, the one or more emitter chamber covers, and the one or more detector chamber covers are fused together.

In some implementations, the light barrier construct, the one or more emitter chamber covers, and the one or more detector chamber covers are chemically bonded together.

In some implementations, the light barrier construct, the one or more emitter chamber covers, and the one or more detector chamber covers are sintered together.

In some implementations, the wearable health monitoring device further comprises a circuit board, wherein the light barrier construct is positioned adjacent to said circuit board, the light barrier construct comprising a plurality of walls forming: one or more emitter chambers, wherein the one or more emitter chamber covers are configured to cover the one or more emitter chambers; and one or more detector chambers spaced from one another and surrounding the one or more emitter chambers, wherein the one or more detector chamber covers are configured to cover the one or more detector chambers. The wearable health monitoring device can further comprise: one or more emitters mounted to the circuit board within said one or more emitter chambers, said one or more emitters configured to emit optical radiation towards tissue of the user's wrist; and one or more detectors mounted to said circuit board within the one or more detector chambers, said one or more detectors configured to detect optical radiation emitted from the first and second emitters after attenuation by said tissue and further configured to output one or more signals based on said detected optical radiation.

Disclosed herein is a method of manufacturing a sensor assembly configured to be integrated into a wearable health monitoring device (such as a watch), the method can include: injection molding one or more emitter chamber covers out of a first material using a first mold; injection molding one or more detector chamber covers out of a second material using a second mold; and injection molding a light barrier construct out of a third material over the one or more emitter chamber covers and the one or more detector chamber covers using a third mold; wherein: the first material includes glass and is optically transmissive; the second material includes glass and is optically transmissive; and the third material includes glass and an optically absorbent pigment.

In some implementations, the first material is transparent, wherein the second material is transparent, wherein the third material is opaque.

In some implementations, the optically absorbent pigment is black.

In some implementations, the method includes coloring the third material with the optically absorbent pigment.

In some implementations, the method includes distributing the optically absorbent pigment throughout the glass of the light barrier construct.

In some implementations, the method includes infusing the glass of the light barrier construct with the optically absorbent pigment.

In some implementations, the first and second materials do not include the optically absorbent pigment.

In some implementations, the first material of the one or more emitter chamber covers is less transparent than the second material of the one or more detector chamber covers to allow the one or more emitter chamber covers to diffuse optical radiation passing therethrough.

In some implementations, the method includes adding texture to the second material of the one or more detector chamber covers to cause the second material to diffuse optical radiation passing therethrough.

In some implementations, the method includes overmolding the light barrier construct over the one or more emitter chamber covers and the one or more detector chamber covers to form a single unitary structure.

In some implementations, the method includes fusing the light barrier construct, the one or more emitter chamber covers, and the one or more detector chamber covers together into a single contiguous mass.

In some implementations, the method includes chemically bonding the light barrier construct, the one or more emitter chamber covers, and the one or more detector chamber covers together into a single contiguous mass.

In some implementations, the method includes sintering the light barrier construct, the one or more emitter chamber covers, and the one or more detector chamber covers with each other to fuse the light barrier construct, the one or more emitter chamber covers, and the one or more detector chamber covers together.

Disclosed herein is a wearable health monitoring device including: one or more emitter chamber covers injection molded out of a first material; one or more detector chamber covers injection molded out of a second material; and a light barrier construct injection molded out of a third material over the one or more emitter chamber covers and the one or more detector chamber covers. In some implementations, the first material is optically transmissive, the second material is optically transmissive, and the third material includes an optically absorbent pigment.

In some implementations, the first material includes glass, wherein the second material includes glass, wherein the third material includes glass.

In some implementations, the first material is transparent, wherein the second material is transparent, wherein the third material is opaque.

In some implementations, the optically absorbent pigment is black.

In some implementations, the third material is colored with the optically absorbent pigment.

In some implementations, the optically absorbent pigment is distributed throughout the light barrier construct.

In some implementations, the third material is infused with the optically absorbent pigment.

In some implementations, the first and second materials do not include the optically absorbent pigment.

In some implementations, the first material of the one or more emitter chamber covers is less transparent than the second material of the one or more detector chamber covers to allow the one or more emitter chamber covers to diffuse optical radiation passing therethrough.

In some implementations, the second material of the one or more detector chamber covers includes texture configured to diffuse optical radiation passing through the second material.

In some implementations, the light barrier construct is overmolded over the one or more emitter chamber covers and the one or more detector chamber covers.

In some implementations, the light barrier construct, the one or more emitter chamber covers, and the one or more detector chamber covers form a single contiguous mass.

In some implementations, the light barrier construct, the one or more emitter chamber covers, and the one or more detector chamber covers are fused together.

In some implementations, the light barrier construct, the one or more emitter chamber covers, and the one or more detector chamber covers are chemically bonded together.

In some implementations, the light barrier construct, the one or more emitter chamber covers, and the one or more detector chamber covers are sintered together.

Disclosed herein is a wearable health monitoring device configured to secure to a wrist area of a wearer, the device including: a housing including a front side and a back side, the back side configured to face tissue of the wearer when the device is worn; and a sensor assembly positioned on the back side of the housing and including: emitters positioned on a surface of a substrate; one or more detectors positioned on the surface of the substrate; a frame positioned adjacent to the surface of the substrate and configured to inhibit transmission of optical radiation through the frame, the frame including: an emitter chamber housing the emitters; and one or more detector chambers housing the one or more detectors; and a substance positioned on the surface of the substrate between the emitters and the one or more detectors, wherein the substance is configured to contact the frame between the emitter chamber and the one or more detector chambers, wherein the substance is configured to inhibit the transmission of optical radiation through the substance between the frame and the surface of the substrate from the emitter chamber to the one or more detector chambers.

In some implementations, the substance forms at least one closed loop on the surface of the substrate.

In some implementations, the substance surrounds the emitters on the substrate.

In some implementations, the substance surrounds the one or more detectors on the substrate.

In some implementations, the substance is opaque.

In some implementations, the substance is adhesive and is configured to adhere to the frame and to the surface of the substrate.

In some implementations, the substance is viscous when applied to the surface of the substrate and solidifies after applied to the surface of the substrate.

In some implementations, the substance includes a compressible foam.

In some implementations, the one or more detector chambers surround the emitter chamber.

In some implementations, each of the one or more detector chambers houses one or more of the one or more detectors.

In some implementations, the frame is opaque and contacts the tissue of the wearer when the device is worn to inhibit transmission of optical radiation along the tissue of the wearer between the emitter chamber and the one or more detector chambers or between the one or more detector chambers.

In some implementations, the frame includes an emitter lens positioned within the emitter chamber and one more detector lenses positioned within the one or more detector chambers, wherein the emitter lens and the one or more detector lenses are configured to transmit optical radiation therethrough.

In some implementations, the emitters form a first group of emitters, wherein the sensor assembly includes a second group of emitters, wherein the frame includes a second emitter chamber housing the second group of emitters, wherein a portion of the substance is positioned on the surface of the substrate between the first group of emitters and the second group of emitters, wherein the substance is configured to contact the frame between the emitter chamber and the second emitter chamber, wherein the substance is configured to inhibit the transmission of optical radiation through the substance between the frame and the surface of the substrate from the emitter chamber to the second emitter chamber.

In some implementations, the substance forms a first closed loop around the emitter chamber and a second closed loop around the second emitter chamber.

Disclosed herein is a wearable health monitoring device configured to secure to a wrist area of a wearer, the device including: a housing including a front side and a back side, the back side configured to face tissue of the wearer when the device is worn; and a sensor assembly positioned on the back side of the housing and including: a first group of emitters positioned on a surface of a substrate; a second group of emitters positioned on the surface of the substrate; one or more detectors positioned on the surface of the substrate; a frame positioned adjacent to the surface of the substrate and configured to inhibit transmission of optical radiation through the frame, the frame including: a first emitter chamber housing the first group of emitters; a second emitter chamber housing the second group of emitters; and one or more detector chambers housing the one or more detectors; and a substance positioned on the surface of the substrate between the first group of emitters and the second group of emitters, wherein the substance is configured to contact the frame between the first emitter chamber and the second emitter chamber, wherein the substance is configured to inhibit the transmission of optical radiation through the substance between the frame and the surface of the substrate from the first emitter chamber to the second emitter chamber.

In some implementations, the substance is opaque.

In some implementations, the substance is adhesive and is configured to adhere to the frame and to the surface of the substrate.

In some implementations, the frame is opaque and contacts the tissue of the wearer when the device is worn to inhibit transmission of optical radiation along the tissue of the wearer between the first and second emitter chambers, between the first and second emitter chambers and the one or more detector chambers, or between the one or more detector chambers.

Disclosed herein is a wearable health monitoring device configured to secure to a wrist area of a wearer, the device including: a housing including a front side and a back side, the back side configured to face tissue of the wearer when the device is worn; and a sensor assembly positioned by the back side of the housing and including: emitters positioned on a surface of a substrate; one or more detectors positioned on the surface of the substrate; and a frame positioned adjacent to the surface of the substrate and configured to inhibit transmission of optical radiation through the frame, the frame including: an emitter chamber housing the emitters and forming an emitter chamber window for optical radiation to pass through; and one or more detector chambers housing the one or more detectors and forming one or more detector chamber windows for optical radiation to pass through, wherein the frame is opaque and configured to directly contact the tissue of the wearer when the device is worn to inhibit transmission of optical radiation between the frame and the tissue of the wearer.

In some implementations, the sensor assembly does not include a light transmissive material positioned between the frame and the tissue of the wearer.

In some implementations, the frame includes one or more edges defining the emitter chamber window and the one or more detector chamber windows, wherein the one or more edges of the frame are configured to contact the tissue of the wearer when the device is worn.

Disclosed herein is a wearable health monitoring device configured to secure to a wrist area of a wearer, the device including: a housing including a front side and a back side, the back side configured to face tissue of the wearer when the device is worn; a sensor assembly positioned by the back side of the housing and including: a first group of emitters positioned on a surface of a substrate and configured to emit optical radiation to the tissue of the wearer; a second group of emitters positioned on the surface of the substrate and configured to emit optical radiation to the tissue of the wearer; and a detector positioned on the surface of the substrate a first distance from the first group of emitters and a second distance from the second group of emitters, wherein the second distance is different than the first distance, wherein the detector is configured to: generate a first signal responsive to detecting optical radiation originating from the first group of emitters after attenuation by the tissue of the wearer, the optical radiation including a first wavelength; and generate a second signal responsive to detecting optical radiation originating from the second group of emitters after attenuation by the tissue of the wearer, the optical radiation including a second wavelength; and one or more hardware processors configured to determine a physiological parameter of the wearer from the first signal and the second signal.

In some implementations, the first wavelength is between 510 nm and 530 nm.

In some implementations, the second wavelength is between 600 nm and 680 nm.

In some implementations, the detector is configured to generate a third signal responsive to detecting optical radiation originating from the second group of emitters after attenuation by the tissue of the wearer, the optical radiation including a third wavelength, wherein the second wavelength is between 600 nm and 640 nm, wherein the third wavelength is between 640 nm and 680 nm.

In some implementations, the second distance is greater than the first distance.

Disclosed herein is a wearable health monitoring device configured to secure to a wrist area of a wearer, the device including: a housing including a front side and a back side, the back side configured to face tissue of the wearer when the device is worn; and a sensor assembly positioned by the back side of the housing and including: a group of emitters positioned on a surface of a substrate; a first group of detectors including one or more detectors positioned on the surface of the substrate; a second group of detectors including one or more detectors positioned on the surface of the substrate; and a frame positioned adjacent to the surface of the substrate and configured to inhibit transmission of optical radiation through the frame, the frame including: an emitter chamber housing the group of emitters; a first detector chamber housing the first group of detectors; and a second detector chamber housing the second group of detectors, wherein the second detector chamber is semi-annular and is symmetrical with the first detector chamber about a first line, wherein the first and second detector chambers surround the emitter chamber.

In some implementations, each of the detectors in the first group of detectors housed by the first detector chamber is separated from the emitter chamber by a respective distance, wherein each of the respective distances is different.

In some implementations, a minimum distance between the first detector chamber and the emitter chamber is between 1 mm and 2 mm.

In some implementations, an optical radiation detecting area of the first detector chamber is between 40 mm² and 50 mm².

In some implementations, the frame includes another emitter chamber housing another group of emitters positioned on the surface of the substrate, the emitter chamber being symmetrical with the another emitter chamber about a second line.

In some implementations, the first line is orthogonal with the second line.

In some implementations, a detector of the first group of detectors is bisected by the second line.

Disclosed herein is a wearable health monitoring device configured to secure to a wrist area of a wearer, the device including: a housing including a front side and a back side, the back side configured to face tissue of the wearer when the device is worn; and a sensor assembly positioned by the back side of the housing and including: a group of emitters positioned on a surface of a substrate; a first group of detectors including one or more detectors positioned on the surface of the substrate; a second group of detectors including one or more detectors positioned on the surface of the substrate; a third group of detectors including one or more detectors positioned on the surface of the substrate; and a frame positioned adjacent to the surface of the substrate and configured to inhibit transmission of optical radiation through the frame, the frame including: an emitter chamber housing the group of emitters; a first detector chamber housing the first group of detectors; a second detector chamber housing the second group of detectors; and a third detector chamber housing the third group of detectors, wherein the first detector chamber, the second detector chamber, and the third detector chamber form an annulus surrounding the emitter chamber.

In some implementations, each of the detectors in the first group of detectors housed by the first detector chamber is separated from the emitter chamber by a respective distance, wherein each of the respective distances is different.

In some aspects, a minimum distance between the first detector chamber and the emitter chamber is between 1 mm and 2 mm.

In some implementations, an optical radiation detecting area of the first detector chamber is between 20 mm² and 35 mm².

In some implementations, the frame includes another emitter chamber housing another group of emitters positioned on the surface of the substrate, the emitter chamber being symmetrical with the another emitter chamber about a line.

In some implementations, a detector of the first group of detectors is bisected by the line.

Various combinations of the above and below recited features, embodiments, implementations, and aspects are also disclosed and contemplated by the present disclosure.

Additional implementations of the disclosure are described below in reference to the appended claims, which may serve as an additional summary of the disclosure.

### BRIEF DESCRIPTION OF THE DRAWINGS

The drawings and the associated descriptions are provided to illustrate aspects of the disclosure and not to limit the scope of the claims. In the drawings, similar elements may have similar reference numerals.

FIGS. 1A-1B illustrates an example wearable device including a sensor assembly worn on a wrist using straps.

FIGS. 2A-2B illustrate perspective views of the example wearable device of FIGS. 1A-1B with a partial view of the straps.

FIG. 2C illustrates a side view of an example wearable device without the straps when the device is interfacing with a wearer's skin.

FIGS. 2D-2E illustrate perspective views of the example wearable device of FIG. 2C.

FIG. 3 illustrates an example wearable device including a sensor assembly worn on a wrist using straps.

FIG. 4 illustrates a schematic system diagram of a wearable device including a sensor assembly.

FIG. 5 illustrates a partially exploded view of an example wearable device.

FIG. 6A illustrates an example light transmissive cover of the sensor assembly of FIG. 5.

FIG. 6B illustrates an exploded view of ECG electrodes, light transmissive cover(s), and an opaque frame of the sensor assembly of FIG. 5.

FIG. 6C illustrates a bottom perspective view of a sensor assembly incorporating the ECG electrodes, light transmissive cover(s), and a opaque frame of FIG. 6A or 6B.

FIG. 7 illustrates a longitudinal cross-sectional view of an example sensor assembly and example light paths between emitters and detectors of the sensor assembly.

FIG. 8A illustrates schematically an example plethysmograph sensor arrangement on a sensor assembly processor board of a sensor assembly of a wearable device.

FIG. 8B illustrates a bottom view of an example sensor assembly incorporating the plethysmograph sensor arrangement of FIG. 8A.

FIG. 8C illustrates a side view of the example sensor assembly of FIG. 8B.

FIG. 8D illustrates a bottom perspective view of the example sensor assembly of FIG. 8B.

FIG. 9A illustrates a bottom view of a variation of the example sensor assembly of FIG. 8B including ECG electrodes.

FIG. 9B illustrates a side view of the example sensor assembly of FIG. 9A.

FIG. 9C illustrates a bottom perspective view of the example sensor assembly of FIG. 9A with the opaque frame and light transmissive cover hidden to show ECG electrodes assembled with the sensor assembly processor board.

FIG. 9D illustrates a bottom perspective view of an example sensor assembly incorporating the plethysmograph sensor arrangement of FIG. 8A.

FIG. 9E illustrates a bottom view of the example sensor assembly of FIG. 9D.

FIG. 9F illustrates a side view of the example sensor assembly of FIG. 9D.

FIG. 10A illustrate schematically an example plethysmograph sensor arrangement on a sensor assembly processor board of a sensor assembly of a wearable device.

FIG. 10B illustrates a bottom view of an example sensor assembly incorporating the plethysmograph sensor arrangement of FIG. 10A.

FIG. 10C illustrates a side view of the sensor assembly of FIG. 10B.

FIG. 11A illustrates a bottom view of an example sensor assembly of a wearable device as worn on a schematic representation of a wearer's wrist.

FIG. 11B illustrates a side view of the sensor assembly of FIG. 11A.

FIGS. 11C and 11D illustrate exploded views of the sensor assembly of FIG. 11A.

FIG. 12A illustrates a bottom view of another example sensor assembly of a wearable device.

FIG. 12B illustrates a side view of the sensor assembly of FIG. 12A.

FIG. 13A illustrates a bottom view of another example sensor assembly of a wearable device.

FIG. 13B illustrates a side view of the sensor assembly of FIG. 13A.

FIG. 14A illustrates a front view of an example aspect of a sensor assembly.

FIG. 14B illustrates an exploded view of an example aspect of a sensor assembly.

FIG. 14C illustrates an example sensor assembly and example light paths between emitters and detectors of the sensor assembly.

FIG. 14D illustrates a perspective view of PCB substrate of a sensor assembly with example plethysmograph sensor arrangement.

FIG. 14E illustrates a longitudinal cross-sectional view of an example sensor assembly.

FIG. 14F illustrates a longitudinal cross-sectional, that is orthogonal to the view of FIG. 14E, of an example sensor assembly.

FIG. 15A illustrates a perspective view of PCB substrate of a sensor assembly with example plethysmograph sensor arrangement.

FIGS. 15B-15C illustrate an example sensor assembly and example light paths between emitters and detectors of the sensor assembly.

FIGS. 15D-15G illustrate an example sensor assembly and example light barriers or light blocks between emitter and detector chambers of the sensor assembly.

FIG. 15H illustrates an example sensor assembly and example light diffusing material and light transmissive lens(es) or cover(s).

FIGS. 16A-16B are front views of a tissue facing portion of a sensor assembly with electrodes.

FIG. 16C is a front view of a tissue facing portion of a sensor assembly without electrodes.

FIGS. 17A-17B are front views of a tissue facing portion of a sensor assembly with electrodes.

FIG. 18A is a front view of an example substrate of a sensor assembly.

FIGS. 18B-18C are side cutaway views of an example sensor assembly.

FIG. 18D is a front view of an interior region of a frame of a sensor assembly.

FIG. 19A is a front view of an example substrate of a sensor assembly.

FIG. 19B is a side cutaway view of an example sensor assembly.

FIGS. 20A-20F illustrate example implementations of a sensor assembly.

FIG. 21 is a front view of a sensor assembly with charging connectors.

FIG. 22 illustrates an example method of manufacturing a housing of a sensor assembly.

FIGS. 23A-23F illustrate various views of an example implementation of emitter chamber covers.

FIGS. 23G-23L illustrate various views of another example implementation of emitter chamber covers.

FIGS. 24A-24E illustrate various views of an example implementation of detector chamber covers.

FIGS. 25A-25G illustrate various views of a frame overmolded with emitter chamber covers and detector chamber covers, according to an example implementation.

FIGS. 26A-26G illustrate various views of a frame overmolded with emitter chamber covers and detector chamber covers, according to another example implementation

FIGS. 27A-27E illustrate various views of a mold assembly for emitter chamber covers.

FIGS. 28A-28E illustrate various views of a mold assembly for detector chamber covers.

FIGS. 29A-29H illustrate various views of a mold assembly for overmolding frames with emitter chamber covers and detector chamber covers.

### DETAILED DESCRIPTION

The present disclosure will now be described with reference to the accompanying figures, wherein like numerals may refer to like elements throughout. The following description is merely illustrative in nature and is in no way intended to limit the disclosure, its application, or uses. Furthermore, the devices, systems, and/or methods disclosed herein can include several novel features, no single one of which is solely responsible for its desirable attributes or which is essential to practicing the devices, systems, and/or methods disclosed herein. Additionally, the structures, systems, and/or devices described herein may be embodied as integrated components or as separate components.

Some aspects and/or implementations have been described in connection with the accompanying drawings. The figures may be drawn to scale, but such scale is not limiting, since dimensions and proportions other than what are shown are contemplated and are within the scope of the disclosed invention. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, any methods described herein may be practiced using any device suitable for performing the recited steps. Various steps within a method may be executed in different order without altering the principles of the present disclosure.

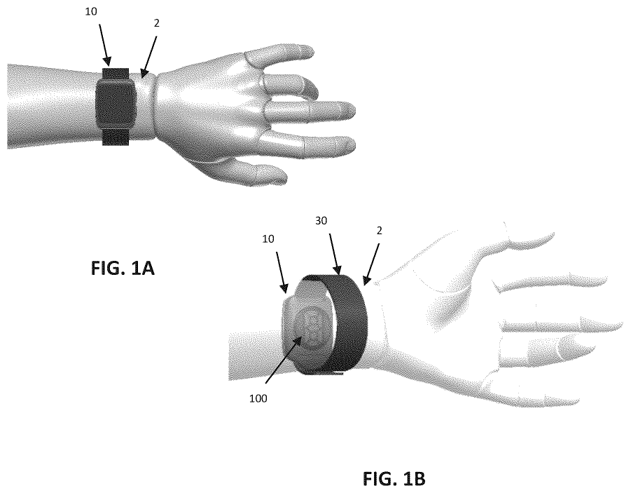

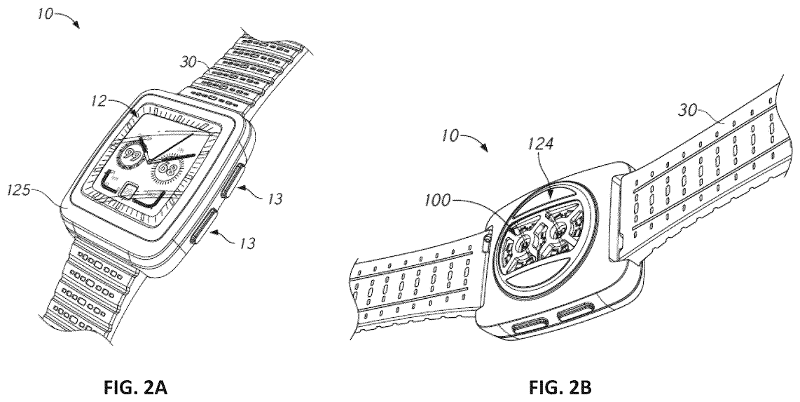

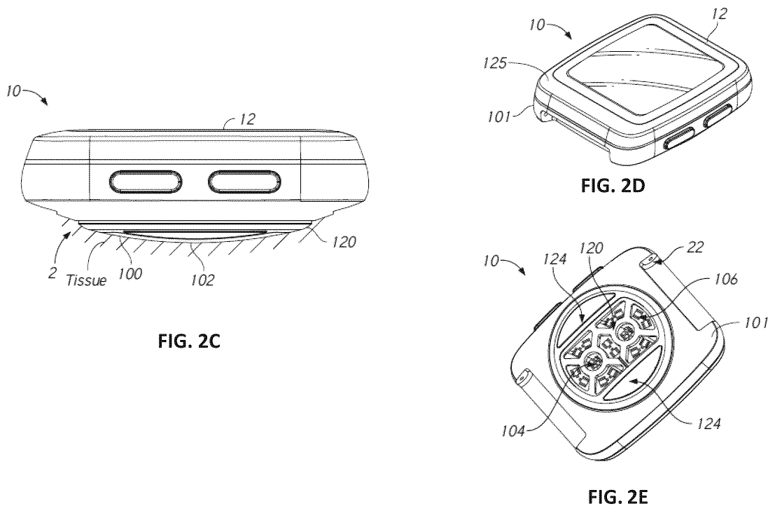

Daily use of a wearable healthcare monitoring device, which can include oximetry- or plethmosmograph-based and/or ECG physiological parameters, can be beneficial to the wearer. The device, such as a device 10 as shown in FIGS. 1A-2E, can be a wristwatch incorporating a sensor assembly 100 or a wrist-worn physiological parameter measurement sensor with built-in watch or time-indicating functions. The device 10 can include an adjustable strap 30. Accordingly, the wearer needs not wear an additional sensor when going about daily activities and the appearance of the device attracts less attention from the general public so that the wearer may feel less self-conscious about wearing a pulse oximeter sensor on the wearer's body. The wearer can also connect additional sensors (for example, a fingertip plethysmograph sensor) and/or other physiological monitoring devices to the wearable device to expand the functionality of the wearable device.

The wearer can be informed of physiological parameters, such as vital signs including but not limited to heart rate (or pulse rate), and oxygen saturation by the wearable device 10. The device 10 can display one or more of the measured physiological parameters on its display 12. The information can be helpful in providing feedback to the wearer and/or a third party user, for example, a healthcare professional or the wearer's family member, when the wearer is exercising, or otherwise for warning the wearer of possible health-related conditions, including but not limited to changes in the wearer's physiological parameters in response to medication that is being administered to the wearer.

As shown in FIGS. 1A-1B and 2A-2E, the wearable device 10 can be a watch, which can include a sensor assembly 100 configured to measure an indication of the wearer's physiological parameters, which can include, for example, pulse rate, respiration rate, oxygen saturation (SpO2), Pleth Variability Index (PVI), Perfusion Index (PI), Respiration from the pleth (RRp), hydration, glucose, blood pressure, and/or other parameters. The sensor assembly 100 can be an optical sensor. Additionally, the sensor assembly 100 can optionally calculate a wellness index based on more than one individual physiological parameter measured by the sensor assembly and/or received by the sensor assembly 100 based on externally connected sensors and/or patient monitoring devices. The sensor assembly 100 can perform intermittent and/or continuous monitoring of the measured parameters. The sensor assembly 100 can additionally and/or alternatively perform a spot check of the measured parameters, for example, upon request by the wearer.

As shown in FIGS. 2B and 2E, a bottom side of a device (or watch) housing 101 can include an opening sized to retain the sensor assembly 100 while still allowing the tissue-facing surface of the sensor assembly 100 to be exposed. The retaining of the sensor assembly 100 in the device housing 101 can be aided by any suitable retaining mechanisms. As shown in FIGS. 2C and 2E, the sensor assembly 100 can include a skin-interfacing light transmissive cover 102 that encloses a plurality of light emitters 104 (such as LEDs) and one or more photodetectors (also referred to as “detectors”) 106. Additionally, the sensor assembly 100 can optionally include an electrocardiogram (ECG) sensor, which can include a plurality of ECG electrodes 124, 125. As shown in FIGS. 2D and 2E, some of the ECG electrodes 125 can be located away from the sensor assembly 100 and some of the ECG electrodes 124 can be located on the sensor assembly 100. The cover 102 can include a plurality of lenses or covers or a single construct of lens or cover. The sensor assembly 100 is designed to reduce noise in the signals detected by the detectors 106, for example, by reducing mixing of the emitted light and the reflected light using light barriers that are substantially opaque. As shown in FIG. 2C, the light barrier construct 120 can include a first light barrier which can be placed between the emitters and the detectors of the sensor assembly 100. The first light barrier can extend (for example, entirely extend) along an inner portion of the cover 102. The first light barrier can also suppress light emitted by the emitters at an angle. The sensor assembly 100 can include additional light barriers, including for example, a side perimeter wall and additional light barriers to separate the detectors from the emitters, and/or separate different detector groups from one another.

FIG. 2C illustrates the device 10 being worn on the wrist 2 of the wearer, with the sensor assembly 100 facing the wrist 2. The sensor assembly 100 on the device 10 is designed so as to reduce and/or eliminate a gap between a surface of the sensor assembly 100 and the wearer's skin at the measurement site where the device 10 is worn. At the wrist, if the device 10 is worn too loosely (which can be the case when the device 10 is able to slide over the skin when the device 10 is moved), the gap between the tissue-facing surface of the sensor assembly 100 and the wearer's skin can cause inaccurate measurements. This is because the gap can result in both light-piping and in the emitted light not penetrating deep enough into the wearer's tissue, for example, by going no deeper than within a top skin layer (for example, the epidermis) of the wearer's tissue, which typically does not have any blood vessels present. Therefore, light cannot reach and or interact with tissues, such as the arterial blood in the dermis, located below the top skin layer. The gap can also result in loss of the attenuated and reflected light through the gap so that less of the attenuated and reflected light can arrive at the detectors 106.

The tightness of the device 10 on the wearer's body (for example, the wrist) can be adjusted by adjusting any suitable strap(s) 30 used to secure the device to the wearer's body. The strap(s) can be connected to the device 10 using any suitable strap connections 22. For example, the strap connections 22 can be compatible with third party watch bands, wearable blood pressure monitors, and/or the like.

Additionally, the gap between a surface of the sensor assembly 100 and the wearer's skin at the measurement site can be reduced by the design of the light transmissive cover 102. As shown in FIG. 2C, a cover 102 of the sensor assembly 100 can include a convex curvature or convex protrusion on its skin-interfacing cover 102. As will be described in greater detail below, the curvature of the cover 102 of the sensor assembly 100, which can include a plurality of lenses or covers or a single lens or cover, can be discontinuous or continuous.

As shown in FIG. 2C, when the device 10 is worn by the wearer, the convex cover 102 can be pressed onto the skin and the tissue 2 of the wearer can conform around the convex curvature. The contact between the convex cover 102 and the tissue 2 of the wearer can leave no air gaps between the tissue 2 and the convex cover 102. And as the emitters and/or detectors can be surrounded by a light-diffusing material (as will be described below), the sensor assembly 100 may leave no air gap between the tissue 2 and any of the emitters and/or detectors. Optionally, certain portion(s) of the cover 102 can protrude more into the skin than the remainder of the cover. The pressure exerted by the curvature of the cover 102 on the skin and/or the absence of air gap can increase a light illuminated and/or detection area, improve the optical coupling of the emitted light and the blood vessels and/or of the reflected light and the detectors, reduce light piping, and/or reduce stagnation of the blood. The cover curvature can be configured so as to balance the pressure needed to improve contact between the cover 102 and the skin, and the comfort of the wearer.

The wearable device 10 can be used in a standalone manner and/or in combination with other devices and/or sensors. The wireless connection can be based on Bluetooth technology, near-field communication (NFC) technology, and/or the like.

Optionally, the device 10 can be integrated with more sensors and/or configured to connect to a plurality of external sensors, wirelessly or with a connecting cable. The connecting cable can be a universal connector configured to connect to any of the medical devices and/or sensors disclosed herein to provide communication between the wearable device 10 and the connected medical devices and/or sensors. The cable can optionally include a board-in-cable device that includes its own processor, but may not include its own display.

The device 10 can act as hub for the external sensors, for example, the sensors described in U.S. Patent Publication No. 2020/0138288, published on May 7, 2020 (the entirety of which is hereby incorporated herein by reference). The sensors described in U.S. Patent Publication No. 2020/0138288 can collect patient physiological data and provide power for a reusable pairing device. The reusable pairing device can establish wireless communication with a patient monitoring device. The wearable device 10 can replace the patient monitoring device in U.S. Patent Publication No. 2020/0138288. As another example, the device 10 can replace a patient monitor device described in U.S. Patent Publication No. 2020/0329993, published on Oct. 22, 2020, the entirety of which is hereby incorporated herein by reference. By replacing the patient monitor device in U.S. Patent Publication No. 2020/0329993, the wearable device 10 performs all the computations based on the sensor data so that the connected external sensors, for example, the ECG sensors disclosed in U.S. Patent Publication No. 2020/0329993, do not require heavy computing power.

The device 10 can include open architecture to allow connection of third party wireless sensor, and/or allow third party access to a plurality of sensors on the wearable device 10 or connected to the wearable device 10. The plurality of sensors can include, for example, a temperature sensor, an altimeter, a gyroscope, an accelerometer, emitters, LEDs, etc. Third party applications can be installed on the wearable device 10 and can use data from one or more of the sensors on the wearable device 10 and/or in electrical communication with the wearable device.

Optionally, the wearable device 10 can communicate with any other suitable noninvasive sensor, such as an acoustic sensor, a blood pressure sensor, temperature sensor, movement sensor, ECG sensor, etc. Examples of some of these devices include Masimo's Radius PPG™ sensor, Radius T™ sensor, and Centroid™ sensor, or otherwise. One or more of those sensors, for example, the Centroid™ sensor, can be used for stroke detection. The wearable device 10 can output an alert of stroke detection of the wearer and/or automatically initiate communication with a first respondent and/or the wearer's guardian or next-of-kin upon stroke detection.

The wearable device 10 can optionally communicate with chemical sensors, which can detect, for example, chemicals on the wearer's skin, and/or sweat, and/or the odor of certain chemicals in the air. The chemical sensors can include electrochemical sensors or any other suitable types of chemical sensors. A chemical sensor configured to analyze compositions of sweat can output measurements aiding the wearable device 10 in detecting stress and/or the wearer's hydration status. The wearable device 10 can optionally communicate with a skin impedance sensor, which can be used for monitoring the hydration status of the wearer.

Another example sensor that can be integrated into or connected to the device 10 and/or the sensor assembly 100 can include a toxin and/or radiation detector configured to detect toxins in air (for example, pollution or contaminant particulates, carbon monoxide, smoke, and the like in the air). The toxin detection can aid care providers and/or firefighters who wear the device 10. Alternatively, the device 10 can be connected wirelessly to an external toxin and/or radiation detector. The toxin and/or radiation detector can be used with a smart mask. For example, the external toxin and/or radiation detector can be located on the mask, which can allow the mask to output a warning to the wearer of the mask when the mask filter or cartridge needs replacement.

Optionally, the wearable device 10 can communicate with glucose monitors, which can be invasive or minimally invasive such as finger prick type of glucose monitors, or a continuous noninvasive glucose monitor. The wearable device 10 can receive and display the wearer's glucose level from the glucose monitor. The wearable device 10 can also optionally be in communication with an insulin pump. The wearable device 10 can send a control signal to dispense insulin from the insulin pump to the wearer based on the monitored glucose level of the wearer.

The sensor assembly 100 can be applied to locations on the body other than the wrist. Alternatively or additionally, multiple sensor assemblies 100 can be applied to different locations of the body of the wearer. Other types of straps or fastening mechanism may be used to attach the multiple sensor assemblies 100 onto other parts of the body. The other types of straps or fastening mechanism can optionally include a power source (for example, battery) to power a sensor assembly 100 that is not integrated into the wearable device 10, but may not have its own display. For example, an optical sensor can be placed on the wearer's neck to measure arterial and venous oxygen saturation, which can be transmitted to and displayed on the wearable device 10. The wearer can view his or her oxygen consumption information on the wearable device 10 based on the signals from the optical sensor on the neck and/or the signals from the sensor assembly 100 that is located on the wearable device 10.

As described above, the sensor assembly can include a plurality of emitters and a plurality of detectors. The emitters can transmit optical radiation of a plurality of wavelengths into a tissue site (near the wrist of the wearer) and the detectors can respond to the intensity of the optical radiation (which can be reflected from the tissue site) after absorption by pulsatile arterial blood flowing within the tissue site. In addition to the light being attenuated by blood in the arteries, light interaction also happens at the capillary level. Arteries are located deeper below the skin surface than the capillaries, requiring LED emitters of greater light intensity and thus greater power consumption in order for the emitted light to reach the arteries. Moreover, measuring the light intensities signal of the light after attenuation by blood in the artery requires more selective placement of the emitters and detectors directly above the arteries to capture the pulsation of the blood. The sensor assembly disclosed herein is designed to utilize attenuation by blood in the capillaries and is not reliant on the blood flow in arteries. The patient parameter measurements made by the sensor assembly disclosed herein can be accurate enough for clinical use. The sensor assembly disclosed herein can provide plethysmograph-based patient parameter measurements with an accuracy of within about 4% error, or about 2% error.

When measuring oxygen saturation based on attenuation by blood in the capillaries, it is desirable to avoid veins. Because venous blood contains less oxygen, intensity signals of light attenuated by venous blood can cause errant readings oxygen saturation measurement. Optionally, the sensor assembly processor of the sensor assemblies disclosed herein can reduce the effect of pulsing vein on the signal by comparing the signals from the plurality of detectors to determine which detectors receive better and/or clearer signals and deactivating the detectors that are more likely to cover and/or be around the pulsing veins. The sensor assembly processor can dynamically adjust which detectors to deactivate. Deactivating the detectors can include deactivating operation of that detector and/or ignoring signals from that detector.

Optionally, the sensor assembly processor of the sensor assembly can map the physiological parameter measurements calculated from signals received at the detectors and/or clusters of detectors located at different regions of the sensor assembly. Variations (for example, if outside a certain range) in the mapped measurements can be an indication that the pressure distribution of the wearable device on the body of the wearer is unbalanced, and therefore the pressure of the device on the wearer is either too high or too low and/or the wearable device is tilted on the wrist. The wearable device can output an instruction to the wearer to readjust the tightness of the straps and/or to re-center of the wearable device on the wrist. Variations (for example, if outside a certain range) in the mapped measurements can additionally or alternatively provide an indication that a certain detector or cluster of detectors is/are placed over a large pulsing vein as described above. Readings from that certain detector or cluster of detectors can be ignored or the detector(s) suspected to be cover a pulsing vein may be deactivated. When two or more physiological parameter measurements, such as oxygen saturation measurements, do not agree among two or more detectors (for example, having a variation exceeding a certain range), the sensor assembly processor can use the higher or highest measurement value, or alternatively use a combination of the measurement values from the two or more detectors (for example, using one of the two measurement values at different times or otherwise).

Alternatively or additionally, the mapped measurements can be compared with experimentally determined data at the same detector location or detector cluster location. The experimentally determined data can be obtained using, for example, a conventional reflectance type pulse oximeter taped over the corresponding detector location, or any other suitable known methods for making the same measurements, including the same wrist-based sensor arrangements described herein. The comparison between the mapped measurements and the experimentally determined data can provide indication of whether the device has achieved a desired pressure on the body of the wearer, whether certain detectors and/or clusters of detectors are placed over or near a pulsing vein, which may interfere with the physiological parameter measurements, or otherwise. For example, if the difference between the mapped measurements and the experimental data at a certain location falls outside a predetermined range, the sensor assembly processor can determine that pressure is too high or too low at that location, and/or that the pressure distribution over the body is not sufficiently balanced to make accurate measurements, and/or a detector or cluster of detectors is/are placed over the wearer's pulsing vein. The experimental data can be stored in a memory device of the sensor assembly processor.

The comparison among the mapped measurements and/or between the mapped measurements and the experimental data can be performed when the wearer first puts on the device and/or at certain time intervals in the duration when the device is worn on the wearer. Additionally, running the comparison-based diagnostics can allow the sensor assembly processor to determine, at the start of the measurement and/or dynamically during use of the device, which detector(s) provide the most accurate and/or reliable measurements.

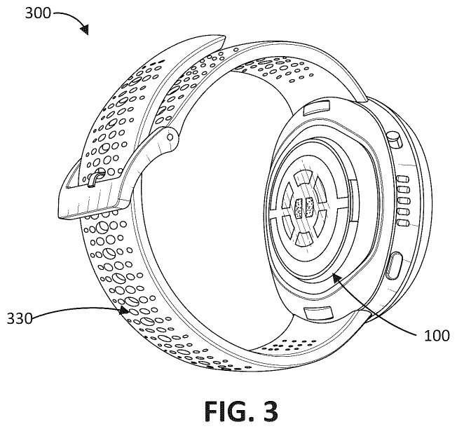

FIG. 3 is a perspective view of an example wearable device 300 which can include similar structural and/or operational features as any of the other wearable devices shown and/or described herein. The device 300 can include one or more straps 330. The device 300 can include a sensor assembly 100 which can include optical emitters and/or detectors within respective chambers, and electrodes which can contact the skin of a user.

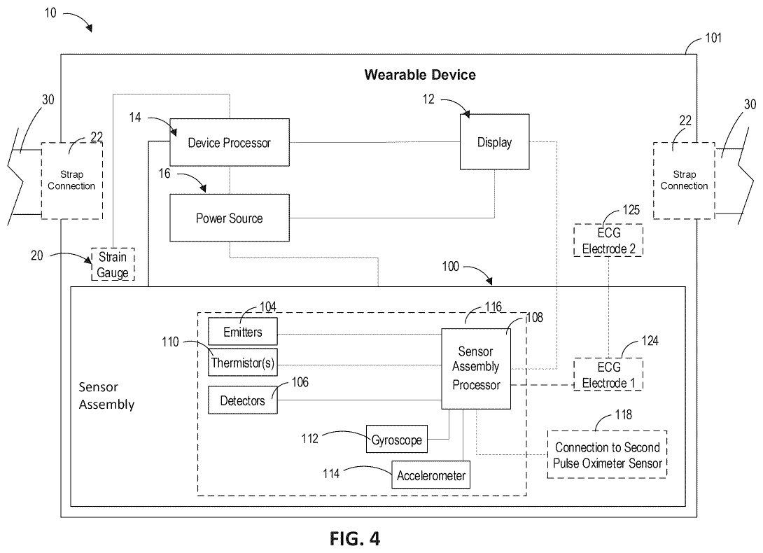

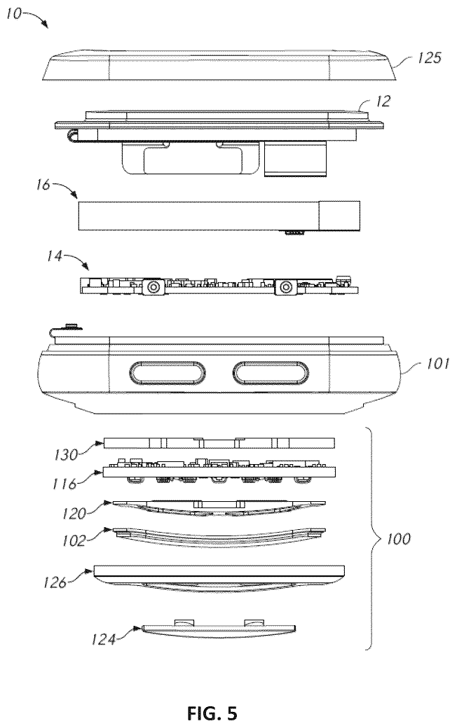

Components of the wearable device will now be described. As shown in FIGS. 4 and 5, the device 10 can include its own device processor 14, which can be a digital/analog chip or other processor(s), such as a digital watch processor or a smartwatch processor. As shown in FIGS. 5, the device processor 14 can be located on a PCB. As shown in FIGS. 4 and 5, the device 10 can include a power source 16, which can be a battery, for powering the device processor 14, the display screen 12, and/or the sensor assembly 100. The battery 16 can last at least 10 hours, or at least 12 hours, or at least 14 hours, or at least about 16 hours after each charge, with continuous measurements and/or displaying of certain physiological parameters, such as SpO2 and pulse rate.

The device 10 can be configured to display time after the battery 16 has been depleted, even if other features (for example, measuring physiological parameters using the sensor assembly) may not be available when the battery 16 has been depleted. Additionally, when the device 10 is used clinically, the display 12 can also continue displaying critical patient information (for example, the patient's name, date of admission, etc.) after the battery 16 has been depleted. The device 10 may include nonvolatile memory to store the critical patient information. The device 10 can include a dual-battery configuration with a main battery and a backup battery. Power management of the device 10 may switch automatically for the device 10 to be powered by the backup battery when the main battery has been depleted. The device can additionally or alternatively be configured to be solar-powered, for example, by including a solar panel on the dial or elsewhere of the wearable device 10. The display 12 of the device 10 can use e-ink or ULP (ultra low power screen) technology, which draws little amount of current for displaying information. The display 12 may automatically adjust the brightness, being brighter when outdoors and dimmer when indoors to further prolong battery life.

As shown in FIGS. 4 and 5, the sensor assembly 100 of the wearable device 10 can include a sensor assembly processor 108. The sensor assembly processor 108 can process signals from one or more of the sensors in the sensor assembly 100 (or optionally other sensors in communication with the device 10) to determine a plurality of physiological parameters. All the processing of the raw sensor data of the sensors in communication (via a wired and/or wireless connection) with the sensor assembly processor 108 is performed by the sensor assembly processor 108. The sensor assembly processor 108 can be configured to drive the emitters 104 to emit light of different wavelengths and/or to process signals of attenuated light after absorption by the body tissue of the wearer from the detectors 106. The sensor assembly processor 108 can determine and output for display on the device display screen 12 the physiological parameters based on the detected signals. Optionally, the sensor assembly 100 can send the signals from the detectors 106 (for example, preprocessed signals) to the device processor 14, which can determine and output for display the physiological parameters based on the detected signals. The absorption of light can be via transreflectance by the wearer's body tissue, for example, by the pulsatile arterial blood flowing through the capillaries (and optionally also the arteries) within a tissue site where the device 10 is worn (for example, the wrist). The sensor assembly processor 108 can be located on a PCB 116, such as shown in FIG. 5.

The sensor assembly 100 can include more than one group or cluster of light emitters (such as LEDs) 104 and more than one group of photodetectors (also referred to as “detectors”) 106. Each group of emitters 104 can be configured to emit four (or three) different wavelengths described herein. The sensor assembly 100 can include one or more thermistors 110 or other types of temperature sensors. The thermistor(s) 110 can be placed near one or more groups of emitters 104. There can be at least one thermistor 110 near each group of emitters 104. The thermistor(s) 110 can provide for wavelength correction of the light emitted by the emitters 104. Optionally, the thermistor(s) 110 can additionally measure a temperature of the wearer of the device 10. Optionally there can be one or more thermistors 110 located at other places of the sensor assembly 100. The emitters 104, the thermistor(s) 110, and/or the detectors 106 can be positioned on the PCB 116.

The emitters 104 of the sensor assembly 340 can be configured to emit a plurality of (for example, three, four, five, or more) wavelengths. The emitters 104 can be configured to emit light of a first wavelength providing an intensity signal that can act as a reference signal. The first wavelength can be more absorbent by the human body than light of other wavelengths emitted by the emitters 104. The reference signal can be used by the sensor assembly processor 348 to extract information from the other signals, for example, information relevant to and/or indicative of the pulsing rate, harmonics, or otherwise. The sensor assembly processor 108 can focus the analysis on the extracted information for calculating the physiological parameters of the wearer. The first wavelength can be between about 500 nm and about 540 nm, between about 505 nm and about 535 nm, between about 510 nm and about 530 nm, between about 515 nm and about 525 nm, or about 520 nm, or any value or range between any of these values or ranges or any value or range bounded by any combination of these values, although values or ranges outside these values or ranges can be used in some cases. The light providing the reference signal can have a green color or alternatively an orange color or yellow color.

The emitters 104 can be configured to emit light of a second wavelength having a red or orange color. The second wavelength can be between about 600 nm and about 640 nm, between about 605 nm and about 635 nm, between about 610 nm and about 630 nm, between about 615 nm and about 625 nm, or about 620 nm, or any value or range between any of these values or ranges or any value or range bounded by any combination of these values, although values or ranges outside these values or ranges can be used in some cases. Light of the second wavelength can be more sensitive to changes in SpO2. The second wavelength is can be closer to 620 nm (for example, about 625 nm or 620 nm), which results in greater absorption by the body tissue of the wearer, and therefore a stronger signal and/or a steeper curve in the signal, than a wavelength that is closer to 660 nm. The sensor assembly processor 348 can extract information such as the pleth waveform from signals of the second wavelength.

The emitters 104 can be configured to emit light of a third wavelength between about 640 nm and about 680 nm, between about 645 nm and about 675 nm, between about 650 nm and about 670 nm, between about 655 nm and about 665 nm, or about 660 nm, or any value or range between any of these values or ranges or any value or range bounded by any combination of these values, although values or ranges outside these values or ranges can be used in some cases.

The emitters 104 can be configured to emit light of a fourth wavelength between about 900 nm and about 910 nm, between about 900 nm and about 905 nm, between about 905 nm and about 910 nm, or about 905 nm, or about 907 nm, or any value or range between any of these values or ranges or any value or range bounded by any combination of these values, although values or ranges outside these values or ranges can be used in some cases. The fourth wavelength can be in the infrared range. The pulse oximeter processor can use the fourth wavelength as a normalizing wavelength when calculating ratios of the intensity signals of the other wavelengths, for example, a ratio of the intensity signals of the second wavelength (red) to the third wavelength (infrared).

Additionally or optionally, the emitters 104 can be configured to emit light having a fifth wavelength that is more sensitive to changes in water than the other emitted wavelengths. The fifth wavelength can be in the infrared range or between about 950 nm and about 990 nm, between about 955 nm and about 985 nm, between about 960 nm and about 980 nm, between about 965 nm and about 975 nm, or about 970 nm, or any value or range between any of these values or ranges or any value or range bounded by any combination of these values, although values or ranges outside these values or ranges can be used in some cases. The sensor assembly processor 108 can determine physiological parameters such as a hydration status of the wearer based at least in part on a comparison of the intensity signals of the fifth wavelength and a different wavelength detected by certain detectors 106.

In some aspects, drivers may drive the emitters at varying intensities. The intensity at which the drivers drive the emitters may affect the amount of light that is outputted (e.g., lumens), the strength of the light signal that is outputted, and/or the distance that the outputted light travels. The drivers may drive the emitters at varying intensities according to modeling, logic and/or algorithms. The logic and/or algorithms may be based, at least in part, on various inputs. The inputs may include historical data, the amount of light that is attenuated, for example as the light penetrates and travels through the tissue of the wearer, or the amount of blood with which the light is interacting, or the type of blood (e.g., venous, arterial) or type of blood vessel (e.g., capillary, arteriole) with which the light is interacting and/or the heat being generated by the emitters. For example, the drivers may increase the intensity at which they drive the emitters based upon a determination that too much light is being attenuated in the tissue or that the light is not interacting with enough blood. As another example, the drivers may decrease the intensity at which they drive the emitters based upon a determination that the emitters have exceeded a threshold temperature. The threshold temperature may be a temperature which may be uncomfortable for human skin.

In some aspects, each of the drivers may be capable of driving a corresponding emitter at various intensities independently of the other drivers. In some aspects, each of the drivers may drive a corresponding emitter at various intensities in unison with each of the other drivers.

Additionally, various LEDs may be used in various aspects. For example, certain LEDs may be used which are capable of outputting more light with the same amount of power as other LEDs. These LEDs may be more expensive. In some aspects, less expensive LEDs may be used. In some aspects, a combination of various types of LEDs may be used.

As shown in FIG. 4, the device 10 can include a gyroscope 112, an accelerometer 114, and/or other position and/or posture detection sensor(s). The gyroscope 112 and/or the accelerometer 114 can be in electrical communication with the sensor assembly processor 108. The sensor assembly processor 108 can determine motion information from signals from the gyroscope 112 and/or the accelerometer 114. The motion information can provide noise reference for analysis of the pleth information and other signal processing (for example, processing of ECG signals) performed by the sensor assembly processor 108. The gyroscope 112 and/or the accelerometer 114 can be located on the PCB 116.

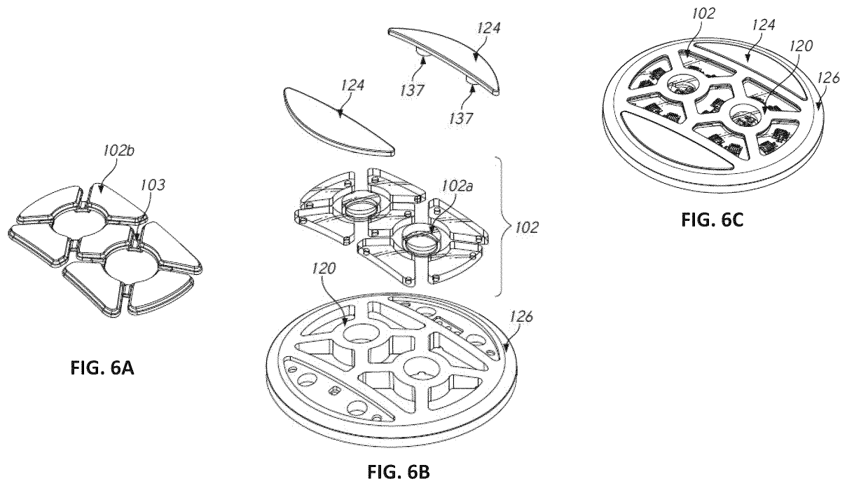

Optionally, as shown in FIGS. 4, 5, and 6B, the device 10 can include an electrocardiogram (ECG) sensor including a plurality of electrodes 124, 125 configured to make contact with the wearer's skin. One or more ECG electrodes 124 may be located on the sensor assembly 100 (such as shown in FIGS. 5 and 6C). One or more ECG electrodes 125 may be located elsewhere on the device (for example, an ECG electrode 125 can form a part of the housing of the wearable device 10 as shown in FIG. 5). The ECG sensor can be in electrical communication with the sensor assembly processor 108 via an ECG connector.

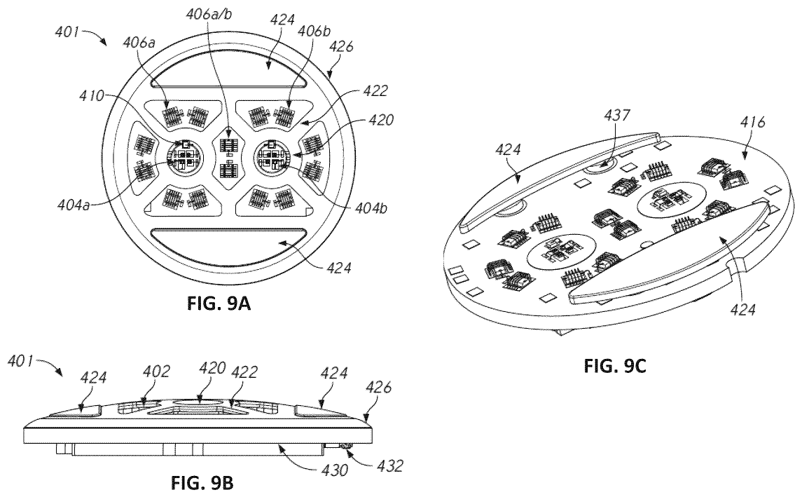

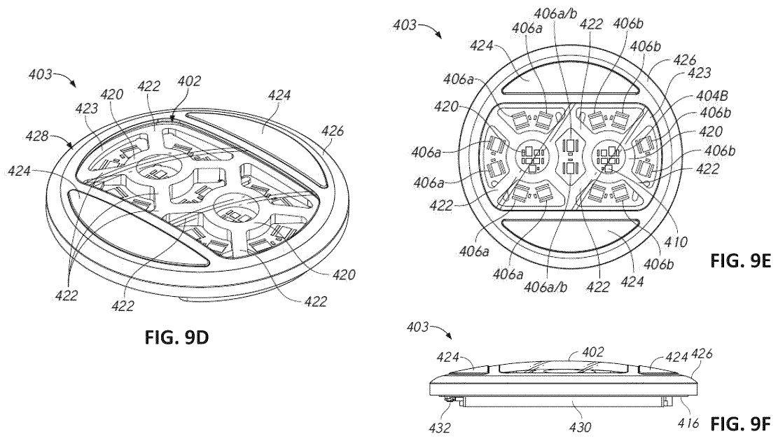

As shown in FIGS. 5-6C, the sensor assembly 100 can include a skin-interfacing light transmissive cover 102 that encloses the first side of the PCB 116, which positions the plurality of light emitters 104 and detectors 106. The sensor assembly 100 can include a light barrier construct 120 that is configured to divide the emitters 104 and the detectors 106 into different chambers such that light cannot travel or substantially cannot travel between the chambers. The light transmissive cover 102 can extend over the various emitter and detector chambers formed by the light barrier construct 120 and the PCB 116. The light transmissive cover 102 can include individual lenses or covers such as shown in FIG. 6B, a single lens or cover such as shown in FIGS. 9D-9F, or a combination of individual emitter chamber covering lenses or covers and a single lens or cover covering a plurality of detector chambers, such as shown in FIG. 6A. In the example lens or cover 102b shown in FIG. 6A, the individual lenses or covers that are configured to cover the detector chambers such as shown in FIG. 6B can be interconnected with bridging portions 103 between the detector chambers, forming a single piece of lens or cover. The lens or cover 102b can be combined with the lenses or covers 102a covering the emitter chambers to cover all the openings in the light barrier construct 120 for forming sealed emitter and detector chambers. The light barrier construct 120 can be overmoulded to the lens or cover 102b and the lenses or covers 102a. The lens or cover 102b may not be configured to cover the emitter chambers, which can be covered by individual lenses, so as to avoid any light traveling between an emitter chamber and a detector chamber.

As shown in FIG. 5, the sensor assembly 100 can include an opaque frame 126. The opaque frame 126 can accommodate the light barrier construct 120. Alternatively, the opaque frame 126 and the light barrier construct 120 can form an integral piece, such as shown in FIG. 6B. The opaque frame 126 can include indentations having the shape and size to accommodate the ECG electrodes 124 or other components with a suitable shape and size. A front side of the electrodes 124 can have one or more posts 137 extending past openings in the opaque frame 126 into corresponding openings on the PCB 116. The posts 137 of the electrodes 124 can establish an electrical connection with the corresponding openings of the PCB 116. A plurality of screws (or other types of fasteners) can extend into the corresponding openings of the PCB 116 from the front side of the PCB 116 to secure the electrodes 124 to the sensor assembly 100 by threadedly mating or otherwise with the posts 137. When a wearer puts the wearable device incorporating the sensor assembly 100 onto the wearer's wrist, the electrodes 124 can make contact with the wearer's skin.

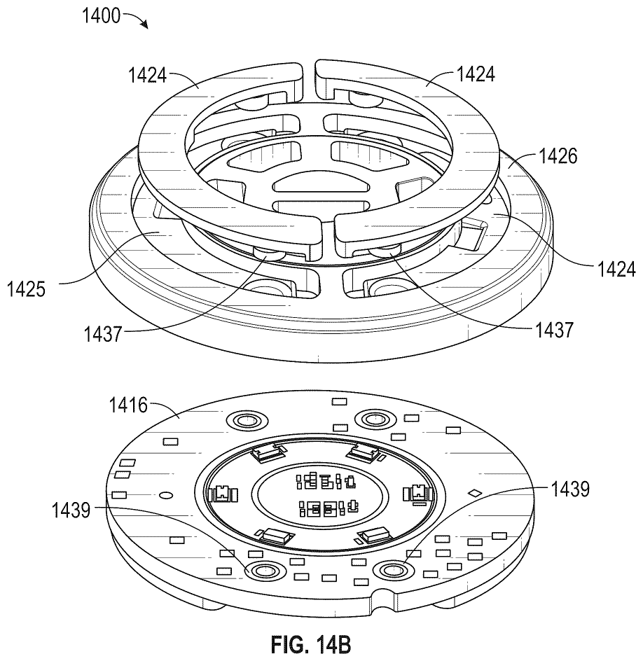

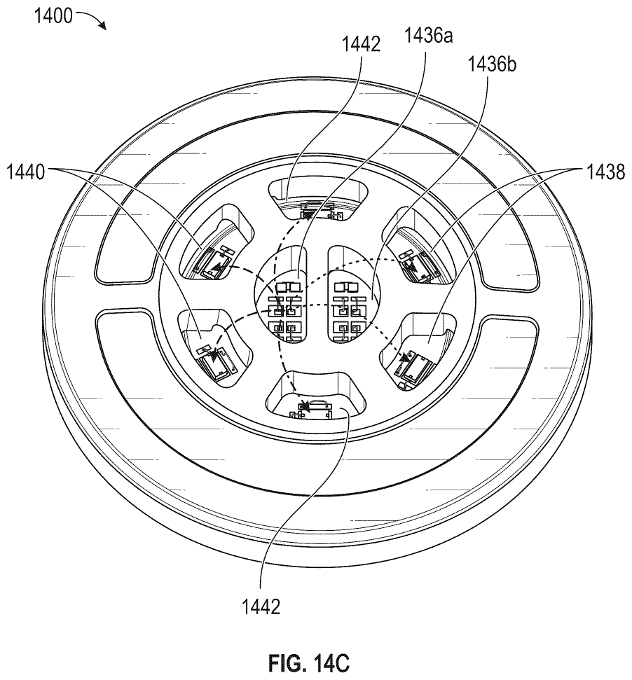

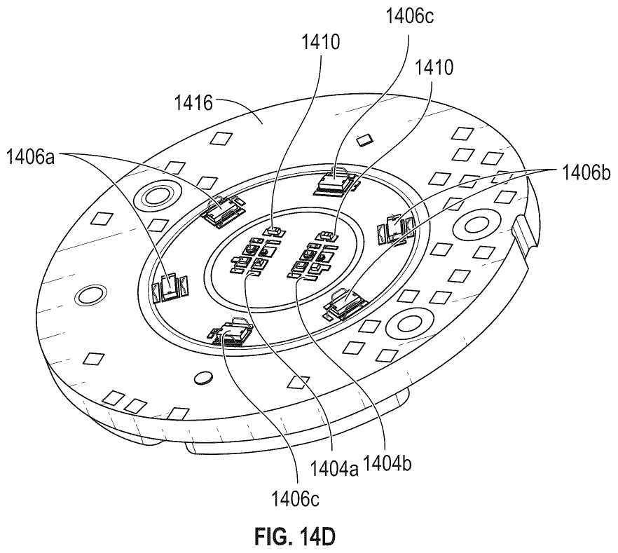

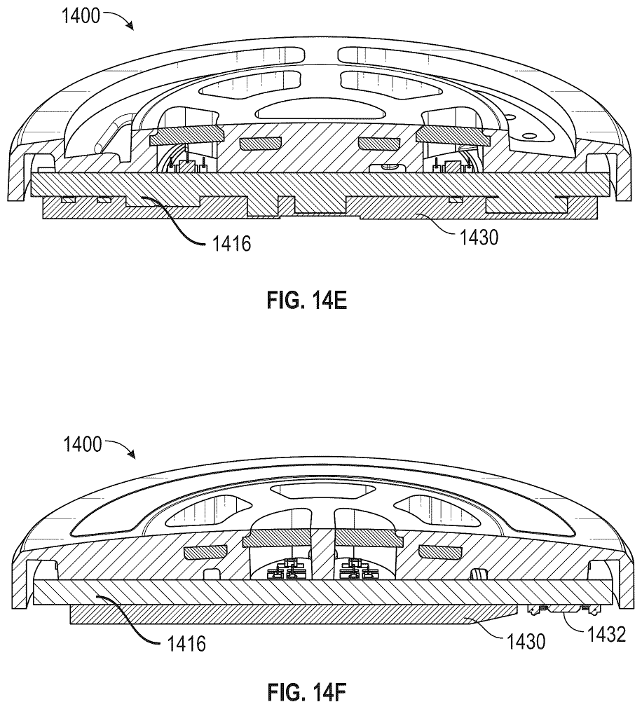

FIGS. 14A, 14B, and 14C illustrate an additional example aspect of an optional electrocardiogram (ECG) sensor. The electrocardiogram (ECG) sensor may include a plurality of electrodes 1424 configured to make contact with the wearer's skin. The plurality of ECG electrodes 1424 may be located on the sensor assembly 1400 (such as shown in FIGS. 14A, 14B and 6C). As disclosed herein, the wearable device incorporating the sensor assembly can include another ECG electrode 125 located on the housing of the wearable device configured to make contact with the wearer's skin.

FIG. 14B is an exploded perspective view of an example aspects of a sensor assembly 1400. As shown in FIG. 14B, the opaque frame 1426 can include recesses (which may also be referred to as “indentations”) having the shape and size to accommodate the ECG electrodes 1424 or other components with a suitable shape and size. For example, in some implementations, frame 1426 includes recesses 1425. Recesses 1425 can be sized and/or shaped to receive ECG electrodes 1424. In some implementations, recesses 1425 have a depth (for example, measured from a plane of the frame 1426) that is substantially equal to a thickness of the ECG electrodes 1424. In some implementations, recesses 1425 have a size and/or shape that matches a size and/or shape of the ECG electrodes 1424. For example, in some implementations in which the ECG electrodes have a semi-annular shape (such as that illustrated in at least FIGS. 14A-14B), the recesses 1425 can have a semi-annular shape.

A front side of the electrodes 1424 can have one or more posts 1437 extending past openings in the opaque frame 1426 into corresponding openings on the substrate 1416. The posts 1437 of the electrodes 1424 can establish an electrical connection with the corresponding openings of the substrate 1416. A plurality of screws (or other types of fasteners) can extend into the corresponding openings of the substrate 1416 from the front side of the substrate 1416 to secure the electrodes 1424 to the sensor assembly 1400 by threadedly mating or otherwise with the posts 1437. When a wearer puts the wearable device incorporating the sensor assembly 1400 onto the wearer's wrist, the electrodes 1424 can make contact with the wearer's skin.

With continued reference to FIG. 14B, the substrate 1416 can include a printed circuit board (PCB). The substrate 1416 can include a conductive liquid adhesive 1439. The conductive liquid adhesive 1439 may be provided on the copper of the substrate 1416. The conductive liquid adhesive 1439 may facilitate conductive electrical connection between the electrodes 1424 and the substrate 1416.

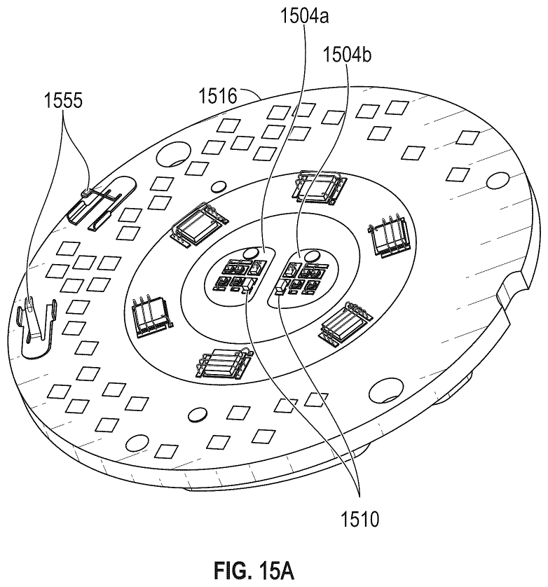

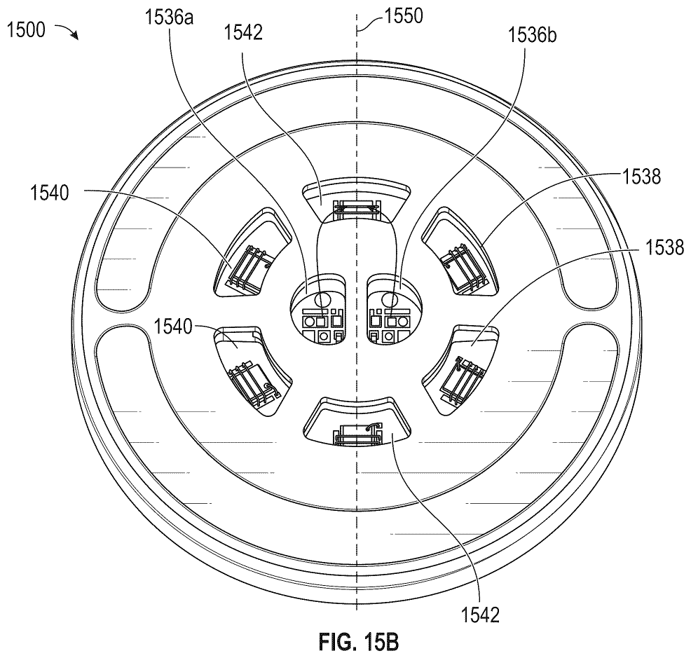

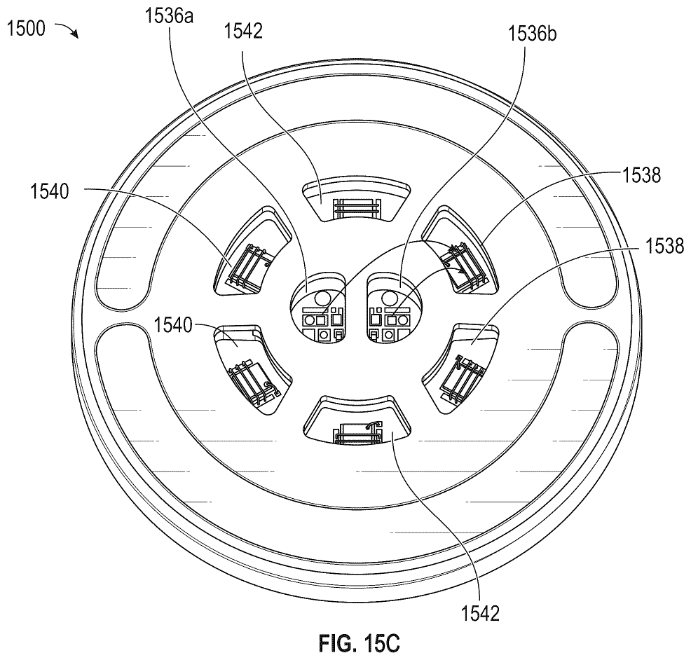

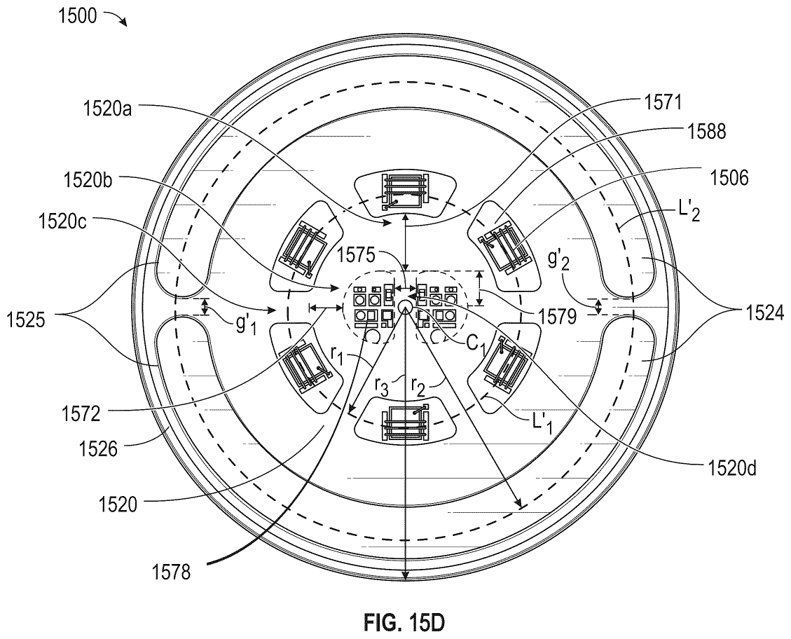

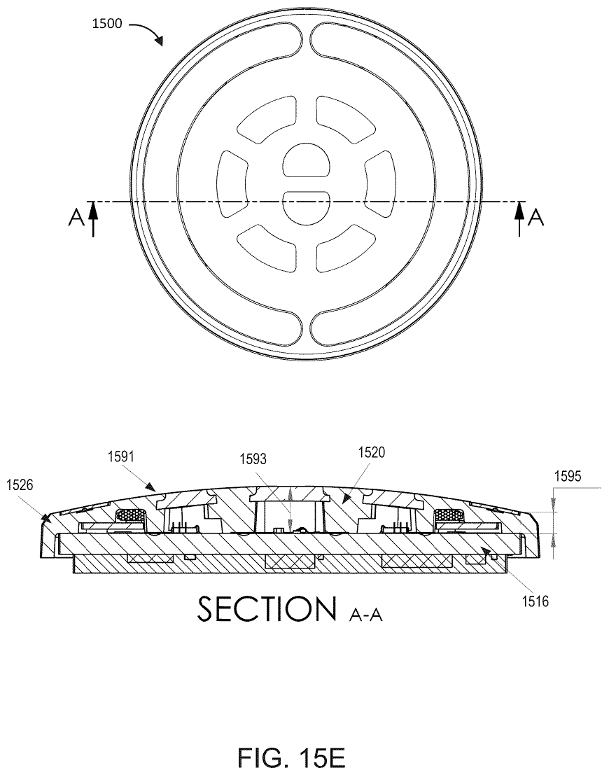

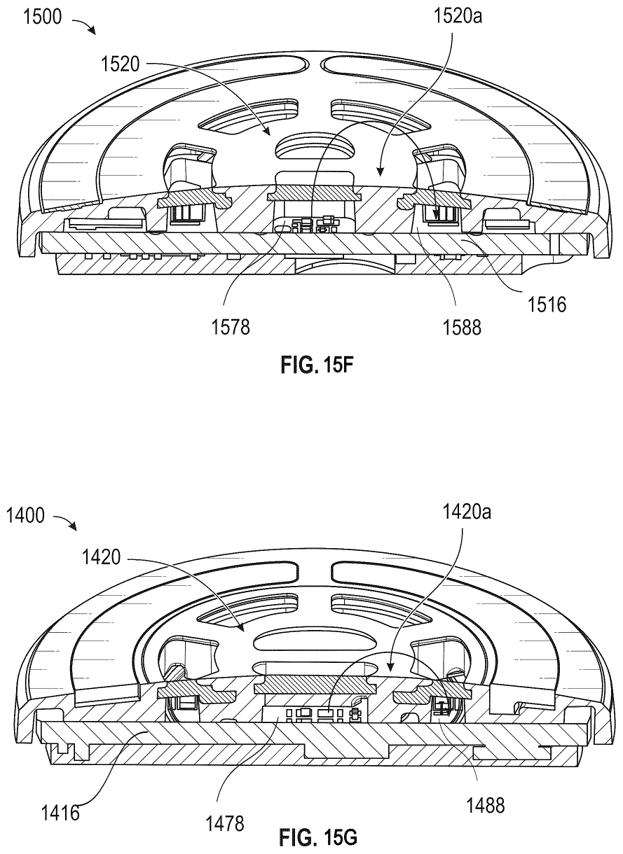

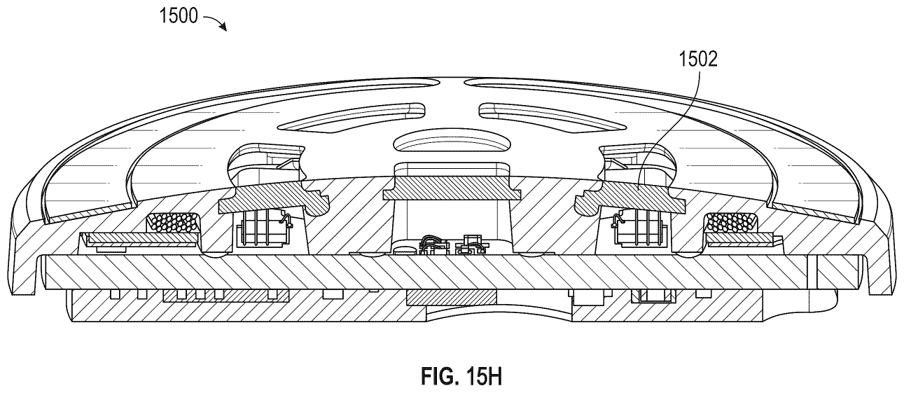

With continued reference to FIG. 14B, one or more spring contacts (such as spring contacts 1555 shown in FIG. 15A) may be located between the electrodes 1424 and the substrate 1416. The shape, size, and/or number of the spring contacts can vary. The spring contacts can establish an electrical connection between the electrodes 1424 and the substrate 1416. The spring contacts can be biased toward the electrodes 1424 to ensure a firm electrical connection between the spring contacts and the electrodes 1424 and the substrate 1416.

The sensor assembly 100 can include diffusing materials or encapsulant, which can include, for example, microspheres or glass microspheres. As described above, the encapsulant can eliminate air gaps between the surface of the light transmissive cover 102 and the emitters 104 and/or the detectors 106. The encapsulant can be included around the emitters 104 to more evenly spread the emitted light, which appears to be emitted from an entire emitter chamber rather than from a point source (that is, a single LED emitter) if the encapsulant is absent. The encapsulant can allow the emitted light to travel through a greater volume of the tissue at the tissue site. The diffusing material can act as a beam shaper that can homogenize the input light beam from the emitter, shape the output intensity profile of the received light, and define the way (for example, the shape or pattern) the emitted light is distributed to a tissue measurement site. Such diffuser materials can, for example, deliver substantially uniform illumination over a specified target area in an energy-efficient manner. According to the Beer-Lambert law, the amount of light absorbed by a substance is proportional to the concentration of the light-absorbing substance in the irradiated solution (for example, the arterial blood). Therefore, by irradiating a larger volume of tissue and/or by increasing the amount of detected light, a larger sample size of light attenuated by the wearer's tissue can be measured. The larger sample size provides a data set that can be more representative of the complete interaction of the emitted light as it passes through the patient's blood as compared to a smaller sample size.

The diffusing materials can be any suitable materials, for example, glass, ground glass, glass beads, opal glass, greyed glass, polytetrafluoroethylene, or a microlens-based, band-limited, engineered diffuser that can deliver efficient and uniform illumination UV-cured flow glass microspheres injected into one or more openings on the sensor assembly 100 (for example, after the sensor assembly 100 has been assembled). Examples of engineered diffusers can include molded plastics with specific shapes, patterns, and/or textures designed to diffuse the emitter light across the entirety of a tissue surface. The diffusing material can be made of ground glass, which spreads the emitted light with a Gaussian intensity profile. The diffusing material can include glass beads. The diffusing material can be constructed so as to diffuse the emitted light in a Lambertian pattern. A Lambertian pattern is one in which the radiation intensity is substantially constant throughout the area of dispersion. One such diffusing material can be made from opal glass. Opal glass is similar to ground glass, but has one surface coated with a milky white coating to diffuse light evenly. The diffusing material can be capable of distributing the emitted light on the surface of a plane (for example, the surface of the tissue measurement site) in a predefined geometry (for example, a rectangle, square, circle, or otherwise), and with a substantially uniform intensity profile and energy distribution. The efficiency, or the amount of light transmitted by the diffusing material, can be greater than 70% of the light emitted by the emitter. The efficiency can be greater than 90% of the emitted light. Additional examples of the diffusing material are described in U.S. Pat. No. 10,448,871, the entirety of which is hereby incorporated herein by reference and should be considered part of the disclosure.

Additionally or alternatively, the sensor assembly 100 can include encapsulant or light diffusing materials in the detector chambers to more evenly spread the reflected light to so as to increase the amount of the reflected light reaching the detectors. The sensor assembly can include light diffusing materials positioned around the detectors to scatter and/or deflect the reflected light so that more reflected light can be detected by the detectors. For example, the reflected light can keep bouncing off the diffusing materials until the reflected light reaches the detector. Accordingly, the light detecting surface area in the sensor assembly can be greater than the surface area of the detectors. Having the light diffusing materials can reduce the power needed to drive the LEDs of the emitters and/or the number of detectors at a particular location of the sensor assembly, which can reduce the power consumption of the sensor assembly.

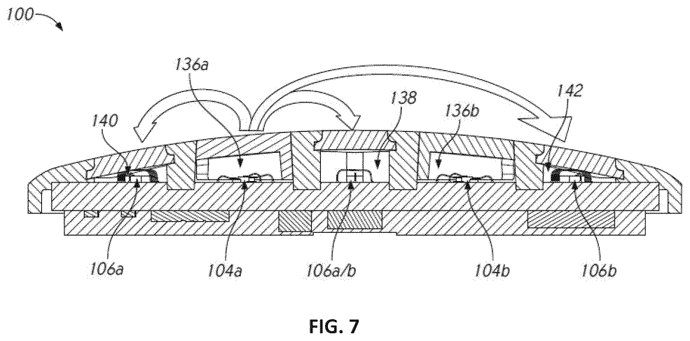

In FIG. 7, a cross-sectional view of the sensor assembly 100 illustrates some of the emitter and detector chambers. The chambers illustrated in FIG. 7 include a first emitter chamber 136a enclosing a first emitter group 104a, a second emitter chamber 136b enclosing a second emitter group 104b, a first detector chamber 140 enclosing one of first groups of detectors 106a that surround the first emitter group 104a, a second detector chamber 142 enclosing one of second groups of detectors 106b that surround the second emitter group 104b, and a third detector chamber 138 enclosing one of shared groups of detectors 106a/b that surround both the first and second emitter groups 104a, 104b on opposite sides of the third detector chamber 138.

As shown in FIG. 7, light from the first emitter group 104a can travel a shorter path, as indicated by the shorter arrows, to the first group of detectors 106a or the shared group of detectors 106a/b; and light from the first emitter group 104a can travel a longer path, as indicated by the longer arrows, to the second group of detectors 106b. The reverse is true for light from the second emitter group 104b, which can travel a shorter path to the second group of detectors 106b or the shared group of detectors 106a/b and a longer path to the first group of detectors 106a. As described herein, the different groups of emitters 104a, 104b and/or detectors 106a, 106b, 106a/b can be run independently and/or simultaneously. Signals outputted by the different groups of detectors 106a, 106b, 106a/b based on light emitted from the first emitter group 104a and/or the second emitter group 104b can provide different information due to the different light paths, which can travel through different areas of the tissue. The longer path penetrates deeper into the tissue and through a greater volume of the tissue to reach the “far” groups of detectors than the shorter path, which penetrates less deep into the tissue and travels through a smaller volume of tissue to reach the “near” group of detectors. The different information can be separated and/or combined to calculate a plurality of physiological parameters of the wearer of the sensor assembly 100, for example, an indication of the wearer's hydration status, which will be described in greater detail below.

FIG. 14C is a front perspective view of an example aspect of a sensor assembly 1400 including another example arrangement of emitter and detector chambers. The emitter and detector chambers may comprise one or more light blocks. The chambers illustrated in FIG. 14C include a first emitter chamber 1436a enclosing a first emitter group, a second emitter chamber 1436b enclosing a second emitter group, a first group of detector chambers 1440, a second group of detector chambers 1442, and a third group of detector chambers 1438. Each detector chamber can enclose one detector. The first and second emitter chambers 1436a, 1436b can be adjacent to each other. The first, second and third groups of detector chambers 1440, 1442, 1438 can extend around the first and second emitter chambers 1436a, 1436b.

As shown in FIG. 14C, light from the first and second emitter groups in the first and second emitter chambers, respectively, can emit light that travel paths of different lengths for example to different detectors. Light from the first emitter group can travel a shorter path, as indicated by the shorter arrows, to the first group of detector chambers 1440; and light from the first emitter group can travel an intermediate path, as indicated by the intermediate arrows, to the second group of detector chambers 1442; and light from the first emitter group can travel a longer path, as indicated by the longer arrows, to the third group of detector chambers 1438. The reverse is true for light from the second emitter group, which can travel a shorter path to the third group of detector chambers 1438, and an intermediate path to the second group of detector chambers 1442, and a longer path to the first group of detector chambers 1440.

As described herein, the different emitters can be run independently and/or simultaneously. For example, the emitters can be selectively activated (e.g., modulated) so that only one emitter (or subset of emitters) is emitting light at a given time. For example, in aspects wherein the first emitter group comprises four emitters, each of the four emitters of the first emitter group may be activated for a quarter cycle (e.g., a different quarter cycle than the other emitters) and off for the remaining three-quarters cycle. For example, a first emitter of the first emitter group may be activated to emit light during only a first quarter cycle, a second emitter of the first emitter group may be activated to emit light during only a second quarter cycle, a third emitter of the first emitter group may be activated to emit light during only a third quarter cycle and a fourth emitter of the first emitter group may be activated to emit light during only a fourth quarter cycle. The emitters of the second emitter group may operate in a similar manner as described.

As another example, in aspects wherein the first emitter group comprises four emitters, each of the four emitters of the first emitter group may be activated for an eighth of a cycle (e.g., a different eighth of the cycle than the other emitters) and off for the remaining seven-eighths cycle. An eighth of a cycle wherein no emitter is activated may occur between each of the cycles wherein an emitter is activated. For example, a first emitter of the first emitter group may be activated to emit light during only a first quarter cycle, a second emitter of the first emitter group may be activated to emit light during only a third quarter cycle, a third emitter of the first emitter group may be activated to emit light during only a fifth quarter cycle and a fourth emitter of the first emitter group may be activated to emit light during only a seventh quarter cycle. The emitters of the second emitter group may operate in a similar manner as described.

The above examples are not meant to be limiting. Alternative activation sequences for the emitters may be used to provide a time-multiplexed signal. In some aspects, the emitters can be selectively activated (e.g., modulated) so that two or more emitters are emitting light at a given time (e.g., during the same cycle or during overlapping cycles), for example in a manner similar to the examples given above.

The emitters may be modulated within an emitter group (e.g., first emitter group and second emitter group) or all of the emitters of the wearable device 10 may be modulated according to a single activation sequence. For example, the emitters of the first group may be modulated according to one activation sequence and the emitters of the second group may be modulated according to a second activation sequence. Alternatively, the emitters of the first and second emitters groups can all be modulated according to a single activation sequence.

In some aspects, the detectors may operate independently from and/or simultaneously with each of the other detectors. For example, each of the detectors may provide an individual signal to the sensor assembly processor 108.

Signals outputted by the different detectors of the different detector chambers 1440, 1442, 1438 based on light emitted from the first emitter group and/or the second emitter group can provide different information due to the different light paths, which can travel through different areas of the tissue. The longer path penetrates deeper into the tissue and through a greater volume of the tissue to reach the detectors of the “far” group of detector chambers than the intermediate and shorter paths. The shorter path penetrates less deep into the tissue and travels through a smaller volume of tissue to reach the detectors of the “near” group of detector chambers than the intermediate and longer paths. The different information can be separated and/or combined to calculate a plurality of physiological parameters of the wearer of the sensor assembly 1400.

For convenience, the terms “proximal” and “distal” are used herein to describe structures relative to the first emitter group or the second emitter group. For example, a detector may be proximal or distal to the first emitter group and may be proximal or distal to the second emitter group. The term “distal” refers to one or more detectors that are farther away from an emitter group than at least some of the other detectors. The term “proximal” refers to one or more detectors that are closer to an emitter group than at least some of the other detectors. The term “intermediate detector” refers to detectors that are closer to an emitter group than distal detectors and farther from an emitter group than proximal detectors. The term “proximal detector” may be used interchangeably with “near detector” and the term “distal detector” may be used interchangeably with “far detector”.

A single detector may be both a proximal to one detector and distal to another detector. For example, a detector may be a proximal detector relative to the first emitter group and may be a distal detector relative to the second emitter group.

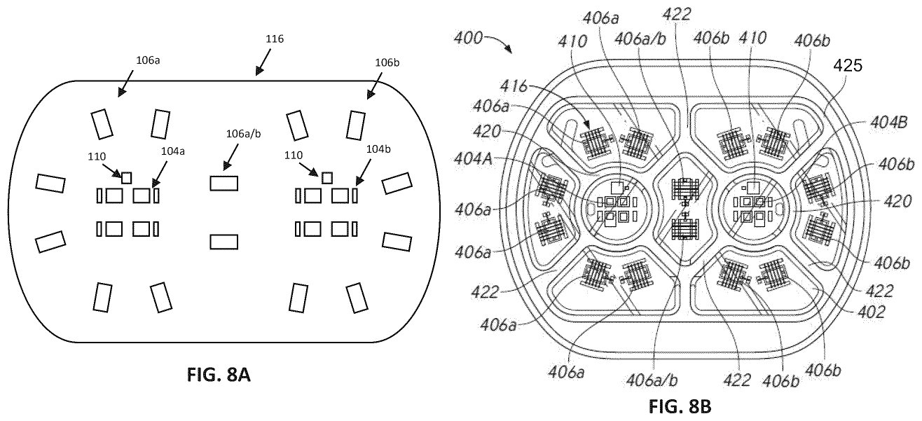

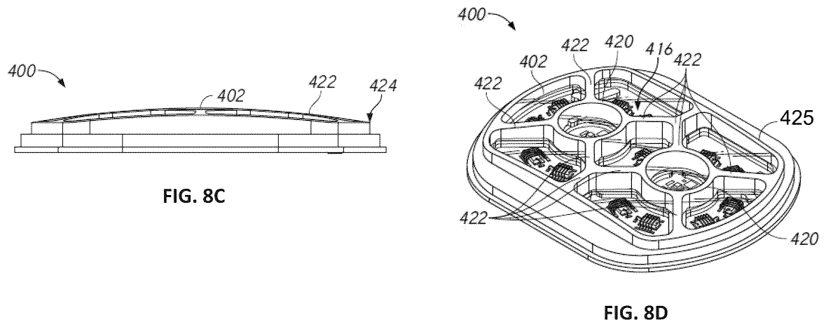

FIG. 8A illustrates schematically an example arrangement of an optical sensor, including emitters, detectors, and thermistors, on a sensor assembly processor PCB 116. As shown in FIG. 8A, the PCB 116 can include a first group of emitters 104a and a second group of emitters 104b. Each group of emitters can include four emitters. The emitters in each group 104a, 104b can emit at least the first, second, third, and fourth wavelengths as described above. The first and second groups of emitters 104a, 104b can be located a distance from each other on a first side of a PCB 116. The PCB 116 can include a temperature sensor (such as a thermistor) 110 as described above located on the first side of the PCB 116. One temperature sensor 110 can be near the first group of emitters 104a. Another temperature sensor 110 can be near the second group of emitters 104b.

The PCB 116 can be elliptical in shape, although the shape of the PCB is not limiting. The two groups of the emitters 104a, 104b can be located on different parts of the first side of the PCB 116 divided along the minor diameter of the ellipse. Each of the two groups of the emitters 104a, 104b can be surrounded by a first light barrier and form an emitter chamber.

The first and second groups of emitters 104a, 104b can be surrounded by two rings of detectors 106a, 106b that are separated from the first and second groups of emitters 104a, 104b respectively by a distance. The two rings of detectors 106a, 106b can share a plurality of (for example, two or more) detectors 106a/b common to both rings. The detectors 106a/b common to both rings can be located along the minor axis of the ellipse. In the illustrated example, the PCB 116 can include fourteen detectors coupled to the PCB 116, but the total number of detectors can vary.

The detectors 106b can be the far detectors for the first group of emitters 104a and the detectors 106a, 106a/b can be the near detectors for the first group of emitters 104a. The detectors 106a can be the far detectors for the second group of emitters 104b and the detectors 106b, 106a/b can be the near detectors for the second group of emitters 104b. Accordingly, each detector 106a, 106b, 106a/b can receive two signals for each wavelength emitted by the first and second groups of emitters 104a, 104b respectively. As described above, signals outputted by the far and near detectors can provide different information due to the different light paths, which can travel through different areas of the tissue. In addition, the far detectors for each group of emitters 104a, 104b can detect the light emitted by the respective group of emitters 104a, 104b, for example, light of the fourth wavelength and another wavelength, and attenuated by tissue to provide an indication of the wearer's hydration status as described herein.

The detectors 106a, 106b, 106a/b can be separated or partitioned into seven detector regions. Each detector region can include two detectors, or any other number of detectors. Each detector region can form a detector chamber surrounded by light barriers. As described above, the sensor assembly processor can process signals from a particular emitter and received at the detectors within the same detector region as one signal source. Accordingly, for each wavelength, the sensor assembly processor can receive data from a total of fourteen signal sources, two from each detector region acting as the far and near detectors for the different groups of emitters respectively.

FIGS. 8B-8D illustrate an example sensor assembly 400 of a wearable device. The sensor assembly 400 can incorporate any of the features of the sensor assembly examples described herein.

As shown in FIG. 8B, the sensor assembly 400 can include a first group of emitters 404a and a second group of emitters 404b incorporating the arrangement shown in FIG. 8A. Each group of emitters can include four emitters (or optionally a different number of emitters, such as six or eight emitters). The emitters in each group 404a, 404b can emit at least the first, second, third, and fourth wavelengths as described above. Each of the two groups of the emitters 404a, 404b can be surrounded by a first light barrier 420 and form an emitter chamber.

The first and second groups of emitters 404a, 404b in the sensor assembly 400 can be surrounded by two rings of detectors 406a, 406b that are separated from the first and second groups of emitters 404a, 404b by the first light barrier 420. The two rings of detectors 406a, 406b can share a plurality of (for example, two or more) detectors 406a/b common to both rings. The detectors 406a, 406b, 406a/b can have the same arrangement as the detectors shown in FIG. 8A. In the illustrated example, the sensor assembly 400 can include fourteen detectors, but the sensor assembly 400 can also include a different total number of detectors.

As shown in FIGS. 8B and 8D, the detectors 406a, 406b, 406a/b can be separated or partitioned into seven detector chambers by a portion of the first light barrier 420 and second light barriers 422. Each detector region can include two detectors, or any other number of detectors. Along an outer perimeter of the sensor assembly 400, the detectors 406a, 406b, 406a/b can be enclosed within a sensor assembly side wall 425. A sensor assembly processor of the sensor assembly 400 can process signals from a particular emitter and received at the detectors within the same detector region as one signal source as described above. The arrangement of emitters 104a, 104b and detectors 106a, 106b, 106a/b and the light diffusing materials encapsulating the emitters 104a, 104b and/or detectors 106a, 106b, 106a/b can improve the sensing coverage on the wearer's wrist, which has fewer capillaries per volume than the fingertip as described above. The aggregate light detecting area of the 106a, 106b, 106a/b in FIG. 8B, that is, the aggregate surface area of all the detector chambers, can occupy about 50% or more of the tissue-facing surface of the sensor assembly. The aggregate light detecting area in FIG. 8B can be, for example, greater than about 100 mm², or greater than about 125 mm², or about 150 mm², or about 165 mm². The aggregate light emitting area in FIG. 8B, that is, the aggregate surface area of both emitters chambers, can be, for example, greater than about 25 mm², or about 30 mm², or about 35 mm². Any other sensor assembly examples disclosed herein can have the same or substantially similar aggregate light detecting area and/or light emitting area as the sensor assembly 400 shown in FIG. 8B.

On the first side of the PCB 416, the sensor assembly 400 can be enclosed by a curved light transmissive cover 402 with a convex protrusion. As shown in FIG. 8C, the cover 402 can have a continuous curvature. The first and second light barriers 420, 422 are configured to be in contact with the first side of the PCB 416 at one end. At the other end, the height of the first and second light barriers 420, 422, and of the side wall 425 can generally follow the curvature of the cover 402. The side wall 425 can be shorter than the second light barrier 422. The height of the second light barrier 422 can increase from the perimeter of the sensor assembly 400 toward a center of the sensor assembly 400 until the second light barrier 422 merges with the first light barrier 420, which is the highest among the light barriers. The light barriers 420, 422 can extend to the tissue-facing surface of the cover 402 so that when the sensor assembly 400 is pressed into the skin of the wearer of a device incorporating the sensor assembly 400, the tissue-facing surfaces of the first and second light barriers 420, 422, and of the side wall 425 can be configured to contact the skin of the wearer. The cover 402 can include individual lenses or covers such as shown in FIG. 6B or a combination of individual emitter chamber covering lenses or covers and a lens or cover covering a plurality of detector chambers, such as shown in FIG. 6A. The lenses or covers may be polycarbonate. The tissue-facing surface of the sensor assembly 400 can include a continuous convex curvature.

The first and second light barriers 420, 422 and the side wall 425 can optionally form a single light barrier construct. The single light barrier construct can be formed by any suitable manufacturing techniques and any suitable materials, for example, plastic, colored, or opaque sapphire glass, or others. The single light barrier construct can include at one end a recess that is shaped and sized to receive the PCB 416, including the electronics on the PCB 416. The first side of the PCB 416 can include the emitters 404a, 404b, detectors 406a, 406b, 406a/b, temperature sensor 410, and any other sensors, for example, the gyroscope, the accelerometer, and/or the like. The second side of the PCB 416 can include the sensor assembly processor and other circuit hardware.

As described above, the sensor assembly 400 can include a plurality of chambers such that light cannot travel between the chambers because of the various light barriers extending from the PCB 416 to the tissue-facing surface of the cover 402 as described herein. The light diffusing materials described above can be added above (for example, via the fill holes described herein) and around the emitters 404a, 404b, and/or optionally above and around the detectors 406a, 406b, 406a/b, to improve distribution of emitted lighted and/or detected light after attenuation by the tissue. The light diffusing materials can include a flow of glass microsphere solution, which can be injected into the chambers after the sensor assembly 400 has been assembled. After being injected into the respective chamber, the solution can be UV-cured. Air can escape via the vent openings disclosed herein as the diffusing material solution is injected into the respective chambers via the injection openings, making it easier for the glass microsphere solution to flow into the respective chamber. The cover 402 can also include glass microspheres. The light diffusing materials in the cover 402 and inside the emitter chambers and/or the first light barrier 420 can make the emitted light leave the emitter chambers enclosing the emitters 404a, 404b in a direction generally parallel to the height of the first light barrier 420. The light diffusing materials in the cover 402 and the detector chambers can increase the amount of reflected light being directed to and detected by the detectors 406a, 406b, 406a/b.

FIGS. 9A-9C illustrate an example sensor assembly 401 of a wearable device. The sensor assembly 401 can include the same optical sensor arrangements as shown in FIGS. 8A-8D and have any of the features of the sensor assembly 400 in FIGS. 8B-8D with the differences noted in the description of FIGS. 9A-9C. The sensor assembly 401 can have any of the features of the other sensor assembly examples described herein.

The sensor assembly 401 can include a generally circular outer shape. The generally circular outer shape can be defined by an opaque frame 426 extending over of the PCB 416 from a first side of the PCB 416. The opaque frame 426 can have a height such that a top side of the opaque frame 426 can be generally level with (or receding or protruding slightly from) a second side of the PCB 416. As shown in FIG. 9C, the PCB 416 can be generally circular in shape. The opaque frame 426 can be generally concentric with the PCB 416. The opaque frame 426 and the PCB 416 are not transmissive to light. The opaque frame 426 in FIGS. 9A and 9B can include the first light barrier 420 and second light barriers 422 as an integral piece.

The sensor assembly 401 can include one or more (for example, two or otherwise) ECG electrodes 424. In the illustrated examples of FIGS. 9A-9C, one of the ECG electrodes 424 can be a reference electrode and the other one of the ECG electrode 424 can be a negative or positive electrode. The opaque frame 426 can have indentations having the shape and size to accommodate the electrodes 424, similar to the indentations on the opaque frame 126 shown in FIG. 6B. As shown in FIG. 9B, a bottom surface of the electrodes 424 can have a curvature that is generally continuous with the curvature of the opaque frame 426 and the light-transmissive cover 402. As shown in FIG. 9C, a top side of the electrodes 424 can have one or more posts 437 extending past openings in the opaque frame 426 into corresponding openings on the PCB 416. The posts 437 of the electrodes 424 can establish an electrical connection with the corresponding openings of the PCB 416. A plurality of screws (or other types of fasteners) can extend into the corresponding openings of the PCB 416 from the front side of the PCB 416 to secure the electrodes 424 to the sensor assembly 401 by threadedly mating with the posts. When a wearer puts the wearable device incorporating the sensor assembly 401 onto the wearer's wrist, the electrodes 424 can make contact with the wearer's skin. The electrodes 424 can have the same polarity as the electrodes 124 disclosed herein. As disclosed herein, the wearable device incorporating the sensor assembly 401 can include another ECG electrode 125 located on the housing of the wearable device configured to make contact with the wearer's skin.

On the second side of the PCB 416, which faces away from the cover 402, the PCB 416 can be covered by melt plastic or other suitable electronics protective material 430 (similar to the protective material 130 disclosed herein) except that a flex connector 432 can remain exposed. The flex connector 432 can be configured to connect the sensor assembly 401 electrically to the wearable device incorporating the sensor assembly 401.

FIGS. 9D-9F illustrate an example sensor assembly 403 of a wearable device. The sensor assembly 403 can include the same optical sensor arrangements as shown in FIGS. 8A-9C and have any of the features of the sensor assembly 400 in FIGS. 8B-8D and any of the features of the sensor assembly 401 in FIGS. 9A-9C with the differences noted in the description of FIGS. 9D-9F. The sensor assembly 401 can have any of the features of the other sensor assembly examples described herein.

As shown in FIGS. 9D-9F, the opaque frame 426 can include an opening fitted with the light transmissive cover 402. The cover 402 extending over emitter chambers or detector chambers formed by the light barriers 420, 422, 423 and the PCB 415 can include a single lens or cover. The cover 402 can be elliptical in shape. The cover 402 can have a continuous convex curvature. As shown in FIG. 9F, the light barriers 420, 422, 423 may not extend to the tissue-facing surface of the cover 402 and can extend to below the cover 402 such that when a wearer puts on a wearable device incorporating the sensor assembly 403, the wearer's tissue comes into contact with the cover 402 and the electrodes 424, but not with any of the light barriers 420, 422, 423.

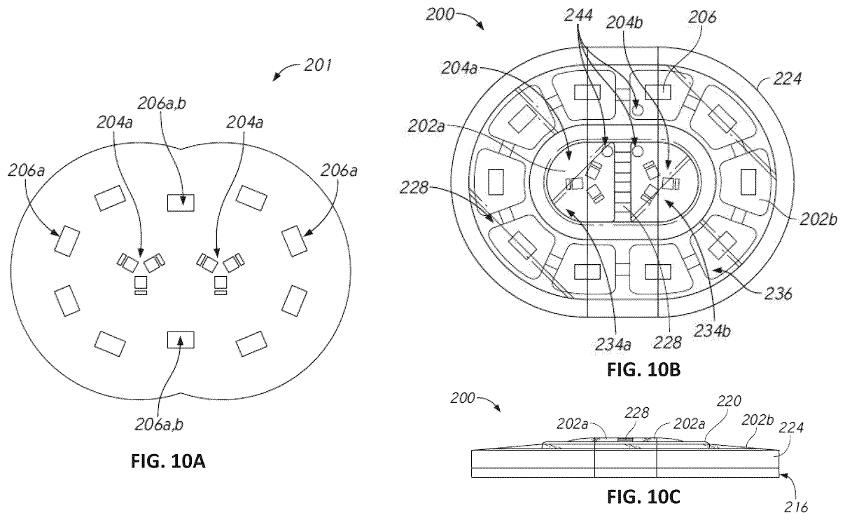

FIGS. 10A-10C illustrate other non-limiting examples of a sensor assembly with two emitter groups in two separate emitter chambers formed by a light barrier. In those configurations, the perimeter of the sensor assembly can have a different shape. For example, FIG. 10A illustrates schematically a sensor assembly 201 having an outer shape of two circles partially overlapped with each other. The circle in the sensor assembly 201 can have a radius, for example, between about 6 mm and about 12 mm, or between about 8 mm and about 10 mm. The sensor assembly 201 can have any of the features of the other sensor assemblies disclosed herein. The sensor assembly 201 can include the substantially the same arrangement of emitters 204a, 204b and detectors 206a, 206b, 206a/b as the sensor assembly 400, 401, 403 described above except that each emitter group 204a, 204b includes three emitters. The sensor assembly 201 can include a thermistor near each emitter group 204a, 204b. The sensor assembly 201 can have a length of, for example, between about 22 mm and about 28 mm, or between about 24 mm and about 26 mm. In some embodiments, the detectors 206a/b may not be located between or separating the two emitter groups 204a, 204b. The sensor assembly 201 can include two groups of emitters that are separated from each other by one light barrier. Each of the detectors in the sensor assembly 201 can form its own detector chamber with one or more light barriers.

The sensor assembly 200 in FIGS. 10B and 10C can have any of the features of the sensor assembly examples described herein. In FIGS. 10B and 10C, a sensor assembly 200 can include two groups of emitters 204a, 204b surrounded by one ring of detectors 206. The sensor assembly 200 can have a width, for example, between about 16 mm and about 22 mm, or between about 18 mm and about 20 mm. The sensor assembly 200 can have a length, for example, between about 20 mm and about 28 mm, or between about 22 mm and about 25 mm.

Each group of the emitters 204a, 204b can include three emitters. Each group of the emitters 204a, 204b can emit at least the first, second, and third wavelength described above. Optionally, each emitter group 204a, 204b can include a fourth emitter configured to emit the fourth wavelength that is more sensitive to water. The emitters can be located at or near a center portion of a PCB 216 of the sensor assembly 200. The sensor assembly 200 can include a temperature sensor located on the PCB 216 near each group of the emitters 204a, 204b.

The emitters can be covered by an inner lens or cover 202a. In the illustrated example, the inner lens or cover 202a can be generally elliptical. In other examples, the inner lens or cover may have any other shapes. The two groups of the emitters 204a, 204b can be located on two parts of the central portion of the PCB divided along the minor diameter of the ellipse. The two groups of the emitters 204a, 204b can be divided by an opaque divider barrier 228, which can reduce mixing of light emitted by the two groups of the emitters 204a, 204b. As shown in FIG. 10C, the divider barrier 228 can have a same or substantially the same height as the highest point of the inner lens or cover 202a when assembled in the sensor assembly 200. The inner lens or cover 202a can include two components divided by the divider barrier 228.

The sensor assembly 200 can include a plurality of detectors 206 (for example, about six, eight, ten, or more) that can be arranged on the PCB so that the detectors 206 are spaced apart around the emitters 204a, 204b. The emitters groups 204a, 204b and the detectors 206 can be separated by a first light barrier 220. The first light barrier 220 can extend along and surround the inner lens or cover 202a. The divider barrier 228 and the first light barrier 220 can form two emitter chambers 234a, 234b, each enclosing one of the two emitter groups 204a, 204b. The first light barrier 220 and the divider barrier 228 can also suppress light emitted by the emitters 204a, 204b at an angle so the light emitted by each group of emitters 204a, 204b can exit the inner lens or cover 202a in a direction generally parallel to the height of the first light barrier 220. The detectors 206 can be enclosed within a sensor assembly side wall 224. The sensor assembly side wall 224 can define a perimeter of the sensor assembly 200. As shown in FIG. 10B, the perimeter of the sensor assembly 200 can have a generally elliptical outer shape. The detectors 206 can be further separated from one another by a plurality of divider barriers 226, forming detector chambers 236, each containing one detector 206.

As shown in FIG. 10C, the first light barrier 220 can protrude slightly from, that is, proud of the edge of the inner lens or cover 202a and the other lenses or covers that will be described below. The detectors 206 can be covered by an outer lens or cover 202b. The outer lens or cover 202b can be generally concentric with the inner lens or cover 202a. In the illustrated examples, the outer lens or cover 202b can be an elliptical disc as shown in FIG. 10B. In other examples such as those disclosed herein, the outer lens or cover can have other shapes. As shown in FIG. 10C, the outer lens or cover 202b can have a smaller curvature than the inner lens or cover 202a such that the inner lens or cover 202a protrudes more than if the inner lens or cover had the same curvature as the outer lens or cover 202b.

As shown in FIG. 10C, the side wall 224 can be shorter than the first light barrier 220. The height of the side wall 224 can be configured such that the tissue-facing end of the side wall 224 is generally continuous with the curvature of outer lenses or covers 202b. The divider barriers 226 can have a height lower than the first light barrier 220. The height of the divider barriers 226 can be configured to accommodate the outer lens or cover 202b such that when assembled, the outer lens or cover 202b forms a substantially smooth surface with the sensor assembly side wall 224. The tissue-facing ends of the first light barrier 220 and the side wall 224, and the tissue-facing surfaces of the inner lens or cover 202a and the outer lens or cover 202b can form the tissue-facing surface of the sensor assembly 200. The slightly protruding first light barrier 220 and/or inner lens or cover 202a can be pressed into the wearer's skin at a higher pressure than the remainder of the lens or cover or light barriers.

The light diffusing materials described above can be included in one or more of the chambers 234a, 234b, 236 of the sensor assembly 200 to improve distribution of emitted lighted and/or detected light. As shown in FIG. 10B, one or more of the lenses or covers 202a, 202b can include an injection opening 244 so that the light diffusing materials, which can include a flow of glass microsphere solution, can be injected into the respective chambers 234a, 234b, 236 after the sensor assembly 200 has been assembled. After the injection, the solution can be UV-cured. The lenses or covers 202a, 202b can include one or more venting openings that are smaller than the injection openings 244. Air can optionally escape via separate vent openings as the diffusing material solution is injected into the respective chambers 234a, 234b, 236 via the injection openings 244. The inner lens or cover 202a and the outer lens or cover 202b can also include glass microspheres so as to act as light diffusers.

FIGS. 14A-15A illustrate other non-limiting examples of a sensor assembly 1400 of a wearable device. The sensor assembly 1400 can incorporate any of the features of the sensor assembly examples described herein. FIG. 14D illustrates an example arrangement of an optical sensor, including emitters, detectors, and thermistors, on a sensor assembly processor substrate 1416. The substrate 1416 can include a printed circuit board (PCB). As shown in FIG. 14D, the substrate 1416 can include a first group of emitters 1404a and a second group of emitters 1404b. Each of the first and second groups of emitters 1404a, 1404b can include four emitters (or optionally a different number of emitters, such as three, six or eight emitters or any other number of emitters as required or desired). Each of the emitters of the first and second groups of emitters 1404a, 1404b may comprise an LED.

Each of the emitters in the first group of emitters 1404a may be located within close proximity to each of the other emitters in the first group of emitters 1404a. For example, each of the emitters of the first group of emitters 1404a may be located on the PCB between 0.2 mm and 2 mm from each of the other emitters in the first group of emitters 1404a. For example, each of the emitters of the first group of emitters 1404a may be located on the PCB about 0.5 mm from each of the other emitters of the first group of emitters 1404a. In some aspects, each of the emitters in the first group of emitters 1404a are positioned such that no more than a certain distance is between each of the emitters in the first group of emitters 1404a, such as 0.5 mm, 1 mm, 1.5 mm and/or 2 mm, or any other distance as required or desired. Each of the emitters in the second group of emitters 1404b may be located within close proximity to each of the other emitters in the second group of emitters 1404b, for example as described above with reference to the first group of emitters 1404a.

Each of the two groups of the emitters 1404a, 1404b can be surrounded by a first light barrier and form emitter chambers for the group of emitters 1404a and group of emitters 1404b, respectively. The first and second groups of emitters 1404a, 1404b can be located a distance from each other on a first side of a substrate 1416. The substrate 1416 can include one or more temperature sensor(s) (such as a thermistor) 1410 as described above located on the first side of the substrate 1416. One temperature sensor 1410 can be near the first group of emitters 1404a within the respective emitter chamber. Another temperature sensor 1410 can be near the second group of emitters 1404b within the respective emitter chamber.

The substrate 1416 can be circular in shape, although the shape of the PCB is not limiting. The two groups of the emitters 1404a, 1404b can be located on different parts of the first side of the substrate 1416 divided along a center line of the circle. Each of the two groups of the emitters 1404a, 1404b can be surrounded by a first light barrier and form an emitter chamber.

The first and second groups of emitters 1404a, 1404b can be surrounded by detectors 1406. As described in greater detail with reference to FIG. 14A, the detectors 1406 can be positioned on the substrate 1416 in a substantially circular or annular arrangement. The detectors 1406 may surround and/or enclose the first and second groups of 1404a, 1404b. Each of the detectors may be a similar or same distance from a geometric center of the substrate 1416 and/or the sensor assembly 1400. In some aspects, the detectors 1406 may be rectangular including longer sides and shorter sides. The detectors 1406 may be positioned on the substrate 1416 such that a long side of each detector is orthogonal to a radius of the substrate 1416. In the illustrated example, the substrate 1416 includes six detectors coupled to the substrate 1416, but the total number of detectors can vary.

The detectors 1406b can be the far detectors for the first group of emitters 1404a. The detectors 1406a can be the near detectors for the first group of emitters 1404a. The detectors 1406a can be the far detectors for the second group of emitters 1404b. The detectors 1406b can be the near detectors for the second group of emitters 1404b. The detectors 1406c can be the intermediate detectors for the first and second groups of emitters 1404a, 1404b. Accordingly, each detector 1406a, 1406b, 1406c can receive two signals for each wavelength emitted by the first and second groups of emitters 1404a, 1404b respectively. As described above, signals outputted by the far, near and intermediate detectors can provide different information due to the different light paths, which can travel through different areas of the tissue.

In some aspects, the sensor assembly processor may evaluate the various signals outputted by the detectors for example by comparing the signal quality of the detectors. The sensor assembly processor may select less than all of the detector signals for processing for each of the far, near and intermediate detectors. For example, the sensor assembly processor may rely on signals from one or two detectors from the four possible far detectors, and one or two detectors from the four possible near detectors, and one or two detectors from the four possible intermediate detectors.

In addition, the far detectors for each group of emitters 1404a, 1404b can detect the light emitted by the respective group of emitters 1404a, 1404b, for example, light of the fourth wavelength and another wavelength, and attenuated by tissue to provide an indication of the wearer's hydration status as described herein.

The detectors 1406a, 1406b, 1406c can be separated or partitioned into six detector regions. Each detector region can include one detector, or any other number of detectors. Each detector region can form a detector chamber surrounded by light barriers. As described above, the sensor assembly processor can process signals from a particular emitter and received at the detectors within the same detector region as one signal source.

The sensor assembly 1400 can include individual lenses or covers or a combination of individual emitter chamber covering lenses or covers and a lens or cover covering a plurality of detector chambers. The lenses or covers may be polycarbonate. The tissue-facing surface of the sensor assembly 1400 can include a continuous convex curvature.

FIG. 15A illustrates another example arrangement of an optical sensor, including emitters, detectors, and thermistors, on a sensor assembly processor substrate 1516. The substrate 1516 of FIG. 15A can include structural and/or operational features similar to those discussed above with reference to FIG. 14D. As shown in FIG. 15A, each of the first and second groups of emitters 1504a, 1504b can include five emitters (or optionally a different number of emitters as required or desired). Each of the emitters of the first and second groups of emitters 1504a, 1504b may comprise an LED and can be configured to emit light at various wavelengths such as any of the wavelengths discussed herein, for example, a first wavelength of about 525 nm to about 650 nm (such as about 525 nm or about 580 nm or about 645 nm), a second wavelength from about 620 nm to about 660 nm (such as about 625 nm), a third wavelength from about 650 nm to about 670 nm (such as about 660 nm), a fourth wavelength from about 900 nm to about 910 nm, and a fifth wavelength at about 970 nm.

As shown in FIG. 15A, the substrate 1516 can include spring contacts 1555 for facilitating physical and/or electrical connection between the substrate 1516 and electrodes (e.g., electrodes 1424 shown in FIG. 14B, for example).

As shown in FIGS. 14E-14F, on the second side of the substrate 1416, which faces away from the cover, the substrate 1416 can be covered by melt plastic or other suitable electronics protective material 1430 (similar to the protective material 130 disclosed herein) except that a flex connector 1432 can remain exposed. The flex connector 1432 can be configured to connect the sensor assembly 1400 electrically to the wearable device incorporating the sensor assembly 1400.

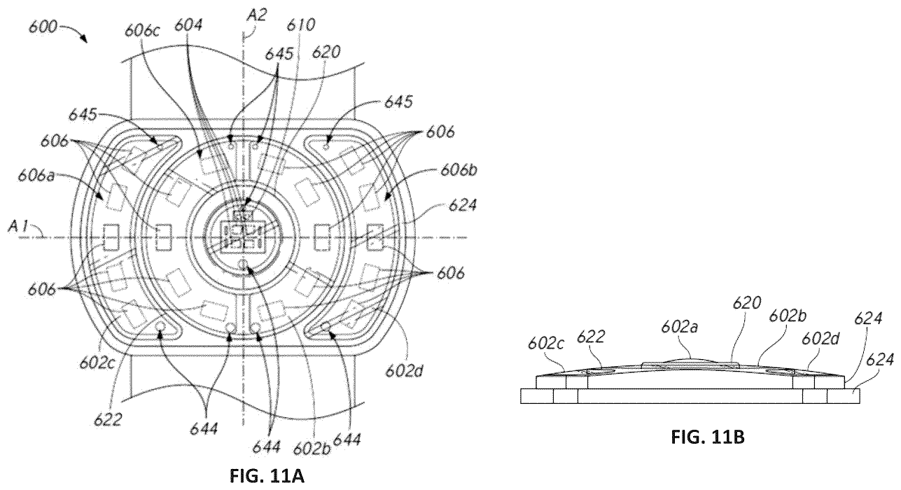

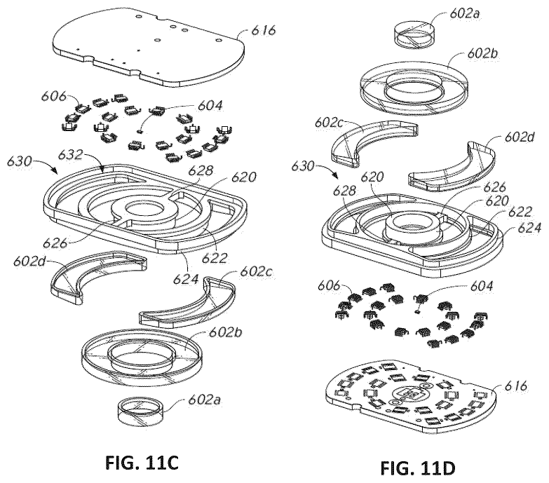

FIGS. 11A-11D illustrate an example sensor assembly 600 of a wearable device. The sensor assembly 600 can have any of the features of the sensor assembly examples described herein, with the differences noted in the description of FIGS. 11A-11D. The sensor assembly 600 can include a single emitter group having a plurality of emitters 604, such as four emitters as shown in FIG. 11A, six emitters, or eight emitters. The emitters 604 of the sensor assembly 600 can emit at least the first, second, third, and fourth wavelengths as described above. The emitters 604 can be located at or near a center portion of a PCB 616 of the sensor assembly 600. The sensor assembly 600 can include a temperature sensor 610 located on the PCB 616 near the emitters 604.

The sensor assembly 600 can include a plurality of detectors 606 that can be arranged on the PCB 616 as an inner group of detectors 606 and an outer group of detectors 606. The inner group 606c of detectors 606, which can include, for example, about ten (or a different number of) detectors 606, can surround the emitters 604 and be spaced apart from one another.

The outer group of detectors 606 can be located further away from the emitters 604 than the inner group of detectors 606. The outer group of detectors 606 can be separated into a first outer group 606a and a second outer group 606b of detectors 606. As shown in FIG. 11A, the sensor assembly 600 can have a first axis A1 and a second axis A2. The outer groups 606a, 606b of detectors 606 can be located further away from the emitters 204 than the inner group of detectors 606 generally along the first axis A1. The two outer groups 606a, 606b of detectors 606 are on opposite sides of the inner group of detectors along the first axis A1. The first and second outer groups 606a, 606b of detectors 606 can be generally symmetrical about the first axis A2 and the second axis A2. Each of the first or second outer groups 606a, 606b of detectors 606 can include about five (or a different number) of detectors 606 that are spaced apart from one another generally along the second axis A2. The outer groups 606a, 606b of detectors 606 can be arranged to be generally concentric with the inner group 606c of detectors 606.

The sensor assembly 600 can be longer in the first axis A1 than in the second axis A2. The sensor assembly 600 can have a dimension of about 25.4 mm (1 inch) along the first axis A1. The sensor assembly can have a dimension of about 19.1 mm (0.75 inch) along the second axis A2. As shown in FIG. 11A, when a watch incorporating the sensor assembly 600 is worn on the wrist of a wearer, the first axis A1 can be generally parallel to the width of the wrist and generally perpendicular to the direction of blood flow along the wrist (that is, along a direction between the hand and the forearm) and the second axis A2 can be generally perpendicular to the width of the wrist and generally parallel to the direction of blood flow along the wrist. The distribution of the detectors 606 along the first axis A1 can improve detection of the light attenuated by the pulsing arterial blood in the capillaries as the detectors 606 are arranged to cover a greater cross-section of the blood flow through the wrist. Similarly, in the other example sensor assemblies described herein, such as the sensor assembly 100, 400, 401, 403, 301, 200, 201, the sensor assembly is incorporated in the wearable device such that the longer side of the sensor assembly is generally perpendicular to the direction of the blood flow along the wrist (see, for example, FIG. 1B) when the wearable device is worn on the wrist.

As shown in FIG. 11A, the emitters 604 can be covered by an inner lens or cover 602a. In the illustrated example, the inner lens or cover 602a can be generally circular. In other examples such as disclosed herein, the inner lens or cover may not be generally circular, but can have other shapes, for example, elliptical, rectangular, square, diamond, or otherwise. The inner group 606c of detectors 606 can be covered by a first outer lens or cover 602b. The first outer lens or cover 602b can be generally concentric with the inner lens or cover 602a. In the illustrated example, the first outer lens or cover 602b can be disc shaped. The first and second outer groups 606a, 606b of detectors 606 can be covered by a second outer lens or cover 602c and a third outer lens or cover 602d respectively. The second and third outer lenses or covers 602c, 602d can be symmetrical about the second axis A2. As shown in FIG. 11B, the first, second, and third outer lenses or covers 602b, 602c, 602d can have substantially the same curvature. The inner lens or cover 602a can be more curved than the outer lenses or covers 602b, 602c, 602d such that the inner lens or cover 602a protrudes more than if the inner lens or cover 602a had same curvature as the outer lenses or covers 602b, 602c, 602d.

The inner group 606c of detectors 606 and the emitters 604 can be separate by a first light barrier 620. The first light barrier 620 can extend along and surround the inner lens or cover 602a, forming an emitter chamber. The first and second outer groups 606a, 606b of detectors 606 can be separated from the inner group 606c of detectors 606 by a second light barrier 622. The second light barrier 622 can be shorter than the first light barrier 620. The first and second outer groups 606a, 606b of detectors 606 can be enclosed within a sensor assembly side wall 624 enclosing a perimeter of the sensor assembly 600. The perimeter of the sensor assembly 600 can be elliptical or any other shape. The side wall 624 can be shorter than the second light barrier 622. The height of the first and second light barriers 620, 622, and of the side wall 624 can generally follow or be substantially continuous with the curvature of the first, second, and third outer lenses or covers 602b, 602c, 602d. The first and second light barriers 620, 622, and of the side wall 624 can have a height so as to be configured to contact the skin of the wearer. Accordingly, the tissue-facing surface of the sensor assembly 600 can be defined by the tissue-facing side of the first and second light barriers 620, 622, and of the side wall 624 and tissue-facing surfaces of the inner lens or cover 602a and the first, second, and third outer lenses or covers 602b, 602c, 602d.

In the illustrated example, the inner group 606c of detectors 606 can be separated by a third light barrier 626 and a fourth light barrier 628 (see FIGS. 11C and 11D). The third and fourth light barriers 626, 628 can have a height lower than the first light barrier 620 or the second light barrier 622. The height of the third and fourth light barriers 626, 628 can be configured to accommodate the first outer lens or cover 602b such that when assembled, the first outer lens or cover 602b forms a substantially smooth surface with the second and third outer lenses or covers 602c, 602d. The first outer lens or cover 602b can sit on top of the third and fourth light barriers 626, 628.

The first light barrier 620 can protrude slightly from, that is, sit proud of the edge of the inner lens or cover 602a and the outer lenses or covers 602b, 602c, 602d. The slightly protruding first light barrier 620 and/or inner lens or cover 602a can be pressed into the wearer's skin at a higher pressure than the remainder of the lenses or covers or light barriers. The first light barrier 620 can also reduce mixing of the emitted and reflected light and/or suppress light emitted by the emitters 604 at an angle so that the emitted light exits the inner lens or cover 602a generally in a direction parallel to the height of the first light barrier 620.

As shown in FIGS. 11C and 11D, the first, second, third, and fourth light barriers 620, 622, 626, 628 and the side wall 624 can optionally form a single light barrier construct 630. The single light barrier construct 630 can be formed by any suitable manufacturing techniques. The single light barrier construct 630 can include at one end a recess 632 (see FIG. 11C) that is configured to receive the PCB 616 (and the emitters 604, detectors 606, temperature sensor 610, and any other sensors, for example, the gyroscope, the accelerometer, and/or the like, and the sensor assembly processor, which are located on the PCB 616). The single light barrier construct 630 can receive the lenses, including the inner lens or cover 602a, the first, second, and third outer lenses or covers 602b, 602c, 602d at another end that is opposite to the end including the recess 632.

The sensor assembly housing can include a plurality of chambers such that light cannot travel between the chambers because of the various light barriers described herein. As described above, the first chamber can be enclosed by the inner lens or cover 602a, the first light battier 620, and a portion of the PCB 616. The first chamber 634 enclose the emitters 604. A second chamber and a third chamber can be enclosed by the first outer lens or cover 602b, the first light barrier 620, the second light barrier 622, the third light barrier 626, the fourth light barrier 628, and a portion of the PCB 616. The second and third chambers can enclose the inner group 606c of detectors 606, with half of the inner group 606c of detectors enclosed by each of the second and third chambers. A fourth chamber can be closed by the second outer lens or cover 602c, the second light barrier 622, the side wall 624, and part of the PCB 616. A fifth chamber can be enclosed by the third outer lens or cover 602d, the second light barrier 622, the side wall 624, and part of the PCB 616. The fourth and fifth chambers can enclose the first and second outer groups 606a, 606b of detectors 606 respectively.

Light from the emitters 604 can travel a shorter path to the inner group 606c of detectors 606 and a longer path to the first and second outer groups 606a, 606b of detectors 606. The inner group 606c of detectors 606 and the first and second outer groups 606a, 606b of detectors 606 can be run independently and/or simultaneously. Signals outputted by the inner and outer groups 606a, 606b of detectors 606 can provide different information due to the different light paths, which can travel through different areas of the tissue. The longer path penetrates deeper into the tissue and through a greater volume of the tissue to reach one of the outer groups 606a, 606b of detectors 606 than the short path, which penetrates less deep into the tissue and travels through a smaller volume of tissue to reach one of the inner group 606c of detectors 606. The different information can be separated and/or combined to calculate a plurality of physiological parameters of the wearer of the sensor assembly 600, for example, an indication of the wearer's hydration status, which will be described in greater detail below.

The light diffusing materials described above can be included in one or more chambers of the sensor assembly 600 to improve distribution of emitted lighted and/or detected light after attenuation by the tissue. As shown in FIG. 11A, one or more of the lenses or covers 602a, 602b, 602c, 602d can include an injection opening 644 so that the light diffusing materials, which can include a flow of glass microsphere solution, can be injected into the respective chambers after the sensor assembly 600 has been assembled. After being injected into the respective chamber, the solution can be UV-cured. The lenses or covers 602a, 602b, 602c, 602d can include one or more venting openings 645 that are smaller than the injection openings 644. Each of the lenses or covers can include at least one venting opening 645. Air can escape via the vent openings 645 as the diffusing material solution is injected into the respective chambers via the injection openings 644, making it easier for the glass microsphere solution to flow into the respective chamber. The inner lens or cover 602a and/or the outer lenses or covers 602b, 602c, 602d can also include glass microspheres. The light diffusing materials in the inner lens or cover 602a and the UV-cured material in the first chamber 634 and/or the first light barrier 620 can make the emitted light leave the first chamber 634 in a direction generally parallel to the height of the first light barrier 620. The light diffusing materials in the outer lenses or covers 602b, 602c, 602d and the UV-cured material in the other chambers 636, 638, 640, 642 can increase the amount of reflected light being directed to the detectors 606.

The sensor assembly examples disclosed herein can monitor a hydration status of the wearer. This is because water in the body tissue can allow a greater portion of the light of the third (or first or second) wavelength disclosed herein to go through (that is, acting as a light pipe), but can bulk absorb the light of the fourth wavelength disclosed herein. The sensor assembly processor can compare intensity signals of the fourth wavelength and another wavelength that is less sensitive to changes in water from the same detector(s). When the wearer's hydration status is in a normal range such that the wearer is not considered dehydrated in a medical sense, the signals of the fourth wavelength and the other wavelength can show opposite trends, that is, one is increasing when the other one is decreasing. When the wearer becomes dehydrated in a medical sense, the opposite trends can become less distinct, for example, by falling below a threshold.

Hydration monitoring can be performed when the sensor assembly, such as the sensor assembly 100, is configured such that at least some of the detectors 106 are located further away (far detector) from one of the emitters 104 or closer to (near detector) the one of the emitters 104. In configurations where there are two emitter groups, each detector 106 or detector region (which can include more than one detector 106 placed enclosed in the same detector chamber) can act as a near (or shallow) detector or detector region for the group of emitters that are closer to that detector 106 or detector region and as a far (or deeper) detector or detector region for the group of emitters that are further away from that detector 106 or detector region.

The sensor assembly 400, 401, 403 illustrates an example configuration for hydration monitoring of the wearer. The detectors 406a can be the far detectors for the second group of emitters 404b and the detectors 406b, 406a/b can be the near detectors for the second group of emitters 404b. The detectors 406b can be the far detectors for the first group of emitters 404a and the detectors 406a, 406a/b can be the near detectors for the first group of emitters 404a.

The sensor assemblies 200, 201 illustrate additional example detectors configurations that can include “near” detectors for one emitter group and “far” detectors for another emitter group, in configurations where the sensor assemblies 200, 201 include a fourth emitter configured to emit light of the fourth wavelength. For example, the detectors 206 on the far side of each group of emitters 204a, 204b can act as “far” detectors for detecting the light emitted by the respective group of emitters 204a, 204b, for example, light of the fourth wavelength and another wavelength, and attenuated by tissue to provide an indication of the wearer's hydration status

The sensor assembly 600 illustrates an example configuration for hydration monitoring of the wearer, with the inner group 606c of detectors 606 acting as the “near” detectors and the outer groups 606a, 606b of the detectors acting as the “far” detectors.

In the above-described configurations, each detector or detector region can provide two measurements calculated from the signals received from the closer emitter group and the signals from the further emitter group respectively. Signals detected at the far detectors can provide indication of the hydration status of the wearer as light travels through a deeper portion of the tissue of the wearer to reach the far detectors than to reach the near detectors). Signals detected at the near detectors can optionally be used as reference or for comparison with the signals detected at the far detectors when the sensor assembly processor determines the wearer's hydration status. The sensor assembly processor of the sensor assembly disclosed herein can compare intensity signals of the fourth wavelength and another wavelength (for example, the third wavelength or about 905 nm) that is less sensitive to changes in water from one of the “far” detectors. The sensor assembly processor can focus on the DC component, or the DC bulk absorption measurement of the signals detected by the “far” detectors for hydration status monitoring. At the DC level, water can act as a light block (that is, less transmissive of light) for the fourth wavelength and as a lens or cover (that is, more transmissive of light) for the other wavelength.

Additionally and/or alternatively, any of the sensor assemblies disclosed herein can monitor the wearer's hydration status by monitoring the wearer's PVI values. The sensor assembly can determine a baseline PVI value of the wearer, and can output a notification that the wearer is dehydrated or hydrated based on fluctuations in the PVI value from the baseline.

The sensor assembly can further combine the hydration status monitoring by the optical detectors and other sensors (such as a sweat sensor or a skin impedance sensors) in outputting a final hydration status indication of the wearer. The sensor assembly can calculate an average, a weight average or otherwise of raw hydration index values calculated based on signals from the different sensors, and/or rely on the different hydration monitoring sensors for redundancy.

As a person's hydration status is not expected to change rapidly, the sensor assembly can optionally make a measurement of the hydration status less frequently than making measurements related to the wearer's pulse rate or SpO2 or other parameters. For example, the sensor assembly processor can make a measurement of hydration status every 5 minutes, or longer, and/or upon (for example, only upon) a request by the wearer, such as when the wearer presses a button (a physical button and/or a touch button on the display) on the device or otherwise instructs the device using voice commands, hand gestures, and/or the like.

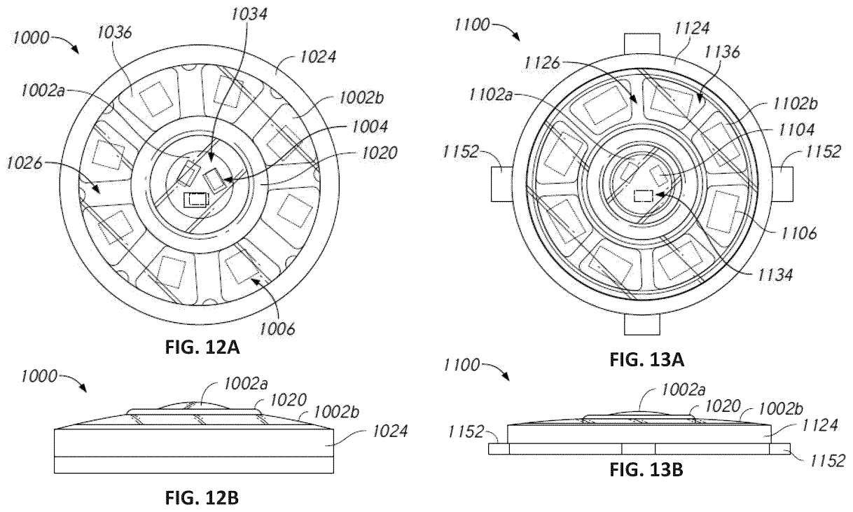

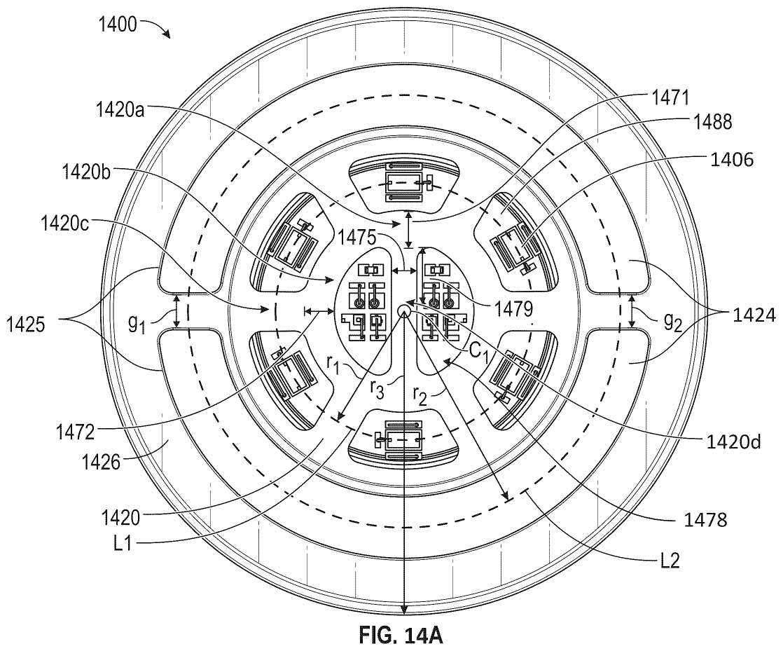

A sensor assembly can alternatively include an inner portion of emitters and an outer ring of detectors as shown in FIGS. 12A-12B and FIGS. 13A-13B. The sensor assembly 1000 in FIGS. 12A-12B and the sensor assembly 1100 in FIGS. 13A-13B can have any of the features of the sensor assembly examples described herein, with the differences noted in the description of FIGS. 12A-12B and 13A-13B. Such a sensor assembly can have a generally circular outer shape. The sensor assembly 1000 in FIGS. 12A-12B can be smaller than the sensor assembly 1100 in FIGS. 13A-13B. For example, the sensor assembly 1000 can have an outer diameter between about 12 mm and about 16 mm, or between about 14 mm and about 15 mm. For example, the sensor assembly 1100 can have an outer diameter between about 16 mm and about 22 mm, or between about 18 mm and about 20 mm.

The sensor assembly 1000, 1100 can each include a single emitter group having a plurality of emitters 1004, 1104, such as three emitters. The emitters 1004, 1104 of the sensor assembly 1000, 1100 can emit at least the first, second, and third wavelengths as described above. The emitters 1004, 1104 can be located at or near a center portion of a PCB of the sensor assembly 1000, 1100. The sensor assembly 1000, 1100 can include a temperature sensor located on the PCB near the emitters 1004, 1104.

The sensor assembly 1000, 1100 can include a plurality of detectors 1006, 1106 (for example, about six, eight, or more) that can be arranged on the PCB so that the detectors 1006, 1106 are spaced apart around the emitters 1004, 1006. The emitters 1004, 1104 and the detectors 1006, 1106 can be separated by a first light barrier 1020, 1120. The first light barrier 1020, 1120 can surround the emitters 1004, 1104. The first light barrier 1020, 1120 can also suppress light emitted by the emitters 1004, 1104 at an angle so that the emitted light exits the inner lens or cover 1002a, 1102a in a direction generally parallel to the height of the first light barrier 1020, 1120.

The emitters 1004, 1104 can be covered by an inner lens or cover 1002a, 1102a. In the illustrated example, the inner lens or cover 1002a, 1102a can be generally circular. The detectors 1006, 1106 can be covered by an outer lens or cover 1002b, 1102b. The outer lens or cover 1002b, 1102b can be generally concentric with the inner lens or cover 1002a, 1102a. In the illustrated examples, the outer lens or cover 1002b, 1102b can be a disc when viewed directly above from the sensor assembly 1000, 1100. In other examples such as those disclosed herein, the outer lens or cover can have other shapes, for example, being elliptical or otherwise. The outer lens or cover 1002b, 1102b can have a smaller curvature than the inner lens or cover 1002a, 1102a such that the inner lens or cover 1002a, 1102a protrudes more than if the inner lens or cover had the same curvature as the outer lens or cover 1002b, 1102b. As shown in FIGS. 12B and 12B, the first light barrier 1020, 1120 can protrude slightly from, that is, proud of the outer edge of the inner lens or cover 1002a, 1102a. The slightly protruding first light barrier 1020, 1120 and/or inner lens or cover 1002a, 1102a can be pressed into the wearer's skin at a higher pressure than the remainder of the light barriers or lenses or covers of the sensor assembly 1000, 1100.

The detectors 1006, 1106 can be enclosed within a sensor assembly side wall 1024, 1124 that defines a perimeter of the sensor assembly 1000, 1100. The perimeter can be generally circular or of any other shape. The side wall 1024, 1124 can be shorter than the first light barrier 1020, 1120. The height of the side wall 1024, 1124 can be such that the tissue-facing end of the side wall 1024, 1124 is generally continuous with the curvature of outer lenses or covers 1002b, 1102b. In the illustrated example, the detectors 1006, 1106 can be separated from one another by a plurality of generally opaque divider barriers 1026, 1126. The divider barriers 1026, 1126 can have a height lower than the first light barrier 1020, 1120. The height of the divider barriers 1026, 1126 can be configured to accommodate the outer lens or cover 1002b, 1102b such that when assembled, the outer lens or cover 1002b, 1102b forms a substantially smooth surface with the sensor assembly side wall 1024, 1124. The outer lens or cover 1002b, 1102b can sit on top of the divider barriers 1026, 1126. The tissue-facing end of the first light barrier 1020, 1120 and the side wall 1024, 1124, and the tissue-facing surfaces of the inner lens or cover 1002a, 1102a and the outer lens or cover 1002b, 1102b can be configured to contact the skin of the wearer and form the tissue-facing surface of the sensor assembly 1000, 1100.

The first light barrier 1020, 1120, the side wall 1024, 1124, and the divider barriers 1026, 1126 can optionally form a single light barrier construct. The single light barrier construct can receive the PCB of the sensor assembly 1000, 1100, and the emitters 1004, 1104, detectors 1006, 1106, temperature sensor, and any other sensors, for example, the gyroscope, the accelerometer, and/or the like, and the sensor assembly processor that are located on the PCB. The single light barrier construct can receive the lenses, including the inner lens or cover 1002a, 1102a and the outer lens or cover 1002b, 1102b on another end that is opposite the end receiving the PCB. As shown in FIGS. 13A and 13B, the light barrier construct of the sensor assembly 1100 or the PCB can additionally include a plurality of (for example, four or otherwise) extension prongs 1152. The plurality of extension prongs 1152 can be generally equally spaced around the side wall 1124.

The sensor assembly 1000, 1100 can include a plurality of chambers such that light cannot travel between the chambers because of the various light barriers described herein. A first chamber 1034, 1134 can be enclosed by the inner lens or cover 1002a, 1102a, the first light battier 1020, 1120, and a portion of the PCB. The first chamber 1034, 1134 can enclose the emitters 1004, 1104. A plurality of second chambers 1036, 1136 can be enclosed by the outer lens or cover 1002b, 1102b, the first light barrier 1020, 1120, the divider barriers 1026, 1126, the side wall 1024, 1124, and part of the PCB. Each of the second chambers 1036, 1136 can enclose one detector 1006, 1106.

The light diffusing materials described above can be included in one or more of the chambers 1034, 1134, 1036, 1136 of the sensor assembly housing to improve distribution of emitted lighted and/or detected light. The inner lens or cover 1002a, 1102a and the outer lens or cover 1002b, 1102b can also include glass microspheres as described above.

FIG. 14A is a front view of an example aspect of a sensor assembly 1400. The sensor assembly 1400 includes an opaque frame 1426, one or more electrodes 1424, one or more detector chambers 1488, one or more emitter chambers 1478, and a light barrier construct 1420.

The opaque frame 1426 can include one or more materials configured to prevent or block the transmission of light. In some aspects, the opaque frame 1426 may form a single integrated unit. In some aspects, the opaque frame 1426 may be formed of a continuous material. The light barrier construct 1420 can include one or more materials configured to prevent or block the transmission of light. In some aspects, the light barrier construct 1420 may form a single integrated unit. In some aspects, the light barrier construct 1420 may be formed of a continuous material. In some aspects, the light barrier construct 1420 and the opaque frame 1426 may form a single integrated unit. In some aspects, the light barrier construct 1420 and the opaque frame 1426 may be separably connected.

The light barrier construct 1420 may include one or more light barriers, such as light barriers 1420a, 1420b, 1420c, 1420d, which are provided as non-limiting examples. In some aspects, light barriers may be also be referred to as light blocks herein. The light barriers may form one or more portions of the light barrier construct 1420. The light barrier construct 1420 (or light barrier portions thereof) may prevent light from passing therethrough. The light barrier construct 1420 may include spaces between various light barriers which may define one or more chambers (e.g., detector chambers 1488, emitter chambers 1478). In some aspects, the one or more chambers (e.g., detector chambers 1488, emitter chambers 1478) may be enclosed by the light barrier construct 1420 or light barrier portions thereof, a surface of a substrate (e.g., PCB), and a lens or cover. In some aspects, light may only enter the chambers through the lens or cover.

An example of a light barrier is provided with reference to example light barrier 1420a. Light barrier 1420a forms a portion of light barrier construct 1420. Light barrier 1420a may prevent (e.g., block) light from passing therethrough between adjacent chambers. For example, light barrier 1420a may prevent light from passing through the light barrier construct 1420 between an emitter chamber 1478 and a detector chamber 1488. Light barrier 1420a, or portions thereof, may include a width 1471. In some aspects, width 1471 may be less than about 1.85 mm. In some aspects, width 1471 may be less than about 1.9 mm. In some aspects, width 1471 may be less than about 1.95 mm. In some aspects, width 1471 may be about 1.88 mm. In some aspects, the width 1471 may be less (e.g., smaller) than length 1479. In some aspects, width 1471 may be less than about 55% of length 1479. In some aspects, width 1471 may be less than about 60% of length 1479. In some aspects, width 1471 may be less than about 65% of length 1479. In some aspects, width 1471 may be about 58.9% of length 1479.

Another example of a light barrier is provided with reference to example light barrier 1420b. Light barrier 1420b forms a portion of light barrier construct 1420. Light barrier 1420b may prevent (e.g., block) light from passing therethrough between adjacent chambers. For example, light barrier 1420b may prevent light from passing through the light barrier construct 1420 between an emitter chamber 1478 and a detector chamber 1488. Light barrier 1420b, or portions thereof, may include a width 1472. In some aspects, width 1472 may be less than about 1.35 mm. In some aspects, width 1472 may be less than about 1.40 mm. In some aspects, width 1472 may be less than about 1.45 mm. In some aspects, width 1472 may be about 1.37 mm. In some aspects, the width 1472 may be substantially similar to width 1471. In some aspects, the width 1472 may be less (e.g., smaller) than width 1471. In some aspects, width 1472 may be less than about 70% of width 1471. In some aspects, width 1472 may be less than about 75% of width 1471. In some aspects, width 1472 may be less than about 80% of width 1471. In some aspects, width 1472 may be about 72.9% of width 1471.

Another example of a light barrier is provided with reference to example light barrier 1420c. Light barrier 1420c forms a portion of light barrier construct 1420. Light barrier 1420c may prevent (e.g., block) light from passing therethrough between adjacent chambers. For example, light barrier 1420c may prevent light from passing through the light barrier construct 1420 between adjacent detector chamber 1488.

Another example of a light barrier is provided with reference to example light barrier 1420d. Light barrier 1420d forms a portion of light barrier construct 1420. Light barrier 1420d may prevent (e.g., block) light from passing therethrough between adjacent chambers. For example, light barrier 1420d may prevent light from passing through the light barrier construct 1420 between adjacent emitter chambers 1478. In some aspects, light barrier 1420d may have a width 1475 separating adjacent emitter chambers of less than about 1.30 mm. In some aspects, width 1475 may be less than about 1.25 mm. In some aspects, width 1475 may be less than about 1.20 mm. In some aspects, width 1475 may be about 1.20 mm. In some aspects, width 1475 may be substantially similar to width 1472. In some aspects, width 1475 may be less (e.g., smaller) than width 1472. In some aspects, width 1475 may be less than about 95% of width 1472. In some aspects, width 1475 may be less than about 90% of width 1472. In some aspects, width 1475 may be less than about 85% of width 1472. In some aspects, width 1475 may be about 87.6% of width 1472.

The emitter chambers 1478 are positioned within a central region of the sensor assembly 1400. The emitter chambers 1478 may be positioned adjacent to one another across a centerline of the sensor assembly 1400 as described in greater detail with reference to FIG. 15B, for example. The emitter chambers 1478 may be positioned adjacent the center point C₁. Each of the emitter chambers 1478 may be a similar size and/or shape. The emitter chambers 1478 may be separated, at least in part, by light barrier 1420d of the light barrier construct 1420. In some aspects, as shown in this example, the light barrier 1420d may form an entire distance between emitter chambers 1478. For example, emitter chambers 1478 may be separated by only the light barrier 1420d such that other components (e.g., detectors, detector chambers, etc.) are not positioned between the emitter chambers 1478.

A portion of the emitter chambers 1478 may extend a length 1479 away from center point C₁. In some aspects, length 1479 may be less than about 3.15 mm. In some aspects, length 1479 may be less than about 3.20 mm. In some aspects, length 1479 may be less than about 3.25 mm. In some aspects, length 1479 may be about 3.19 mm. In some aspects, the length 1479 may be greater (e.g., larger) than a width of a light barrier separating an emitter chamber from a detector chamber such as width 1471. In some aspects, length 1479 may be greater than about 165% of width 1471. In some aspects, length 1479 may be greater than about 170% of width 1471. In some aspects, length 1479 may be greater than about 175% of width 1471. In some aspects, length 1479 may be about 169.7% of width 1471.

As shown in this example aspect, the detector chambers 1488 are arranged in a substantially circular pattern. Each of the detector chambers 1488 houses a detector 1406 positioned on a substrate (e.g., PCB) in a substantially circular or annular pattern. The detectors 1406 may be positioned in a central region of each of the respective detector chambers 1478. The detector chambers 1488 are arranged along a ring defined by ring L₁. In some aspects, such as shown in this example aspect, detectors 1406 of respective detector chambers 1488 may also be arranged along a same ring along which the detector chambers 1488 are arranged (such as in aspects where detectors are positioned in a central region of respective chambers). The ring L₁ may intersect a central region of the detector chambers 1488. In this example aspect, the ring L₁ encloses an entirety of the emitter chambers emitter chambers 1478 such that the emitter chambers 1478 are positioned within an interior region (e.g., a central region) of the ring L₁ defined by the detector chambers 1488. In some aspects, each of the detector chambers 1488 (and corresponding detectors 1406 within respective detector chambers 1488) may be positioned at a substantially similar or same distance away from the center point C₁ (e.g., center of sensor assembly 1400). In some aspects, the detectors 1406 may be rectangular including longer sides and shorter sides. The detectors 1406 may be positioned on a substrate of the sensor assembly 1400 such that a long side of each detector is orthogonal to a radius extending away from center point C₁ (e.g., radius r₁, radius r₂, radius r₃). Advantageously, orienting the detectors 1406 on the sensor assembly 1400 in an annular arrangement with a long side of the detectors 1406 orthogonal the center point C₁ may improve an accuracy of physiological measurements by ensuring that light from emitters travels along a known path length from emitters to the detectors 1406 and may also reduce processing requirements of the sensor assembly 1400 by reducing the amount of variables (e.g., number of light path lengths) required to process in order to determine physiological data.

The electrodes 1424 can include a reference electrode and a negative electrode (and/or a positive electrode). In some aspects, a wearable device such as a watch incorporating the sensor assembly 1400 can include another ECG electrode (e.g., a positive electrode) located on the housing of the wearable device configured to make contact with the wearer's skin. In some configurations, a surface of the electrodes 1424 may be flush with a surface of the opaque frame 1426.

The electrodes 1424 are positioned within or along a portion of the opaque frame 1426 such as shown in FIG. 14B for example. In some aspects, the electrodes 1424 can be substantially semicircular. In some aspects, the electrodes 1424 can be substantially semiannular. In the example aspect shown, each of the electrodes 1424 forms a substantial half annulus. Advantageously, an annular shaped electrode may improve contact with the skin of a wearer (e.g., by contacting a diverse area of skin) while simultaneously reducing the amount of surface area of the electrode. In some aspects, each of the electrodes 1424 may be a similar size and/or shape. In some aspects, the electrodes 1424 may be various sizes and/or shapes. In this example aspect, the electrodes 1424 are positioned within the sensor assembly 1400 (e.g., within the opaque frame 1426) along ring defined by L₂. In various aspects described herein, the ring L₂ may include various radii which may advantageously provide improved contact between the electrodes 1424 and the skin of a wearer of the device.

The opaque frame 1426 includes one or more gaps (e.g., g₁, g₂) between electrodes 1424. The gaps g₁, g₂, (or other portions of the opaque frame 1426) may electrically insulate each of the electrodes 1424 from one another. Each of the electrodes 1424 includes substantially straight edge along a portion of respective gaps g₁, g₂. In some aspects, the gaps g₁, g₂, may be a similar or a same size. In some aspects, the gaps g₁, g₂, may be a different size than each other. In some aspects, the gaps g₁, g₂, may be less than about 1.6 mm. In some aspects, the gaps g₁, g₂, may be less than about 1.65 mm. In some aspects, the gaps g₁, g₂, may be less than about 1.7 mm. In some aspects, the gaps g₁, g₂, may be about 1.62 mm. As discussed above, in some implementations the frame 1426 includes recesses 1425 sized and/or shaped to receive the ECG electrodes 1424. In some implementations, each of such recesses 1425 includes first and second ends, the first ends of the recesses 1425 are separated from one another by gap g₁, and the second ends of the recesses 1425 are separated from one another by gap g₂.

The ring L₁ may be concentric with an outer perimeter of the sensor assembly 1400. The ring L₂ may be concentric with an outer perimeter of the sensor assembly 1400. The ring L₂ may be concentric with a ring defined by positions of the detector chambers 1488 such as ring L₁. Center point C₁ may define a geometric center of ring L₁. Center point C₁ may define a geometric center of ring L₂. Center point C₁ may define a geometric center of an outer perimeter of the sensor assembly 1400. In some aspects, such as shown in FIG. 14A, each of L₁, L₂, and an outer perimeter of the sensor assembly 1400 are concentric with each other and share a same geometric center shown as C₁.

The ring L₁ may include a radius r₁. In some aspects, radius r may be less than about 6.25 mm. In some aspects, radius r₁ may be less than about 6.50 mm. In some aspects, radius r₁ may be less than about 6.75 mm. In some aspects, radius r₁ may be about 6.34 mm. In some aspects, the radius r₁ may be less (e.g., smaller) than radius r₂. In some aspects, radius r₁ may be less than about 55% of r₂. In some aspects, radius r₁ may be less than about 60% of r₂. In some aspects, radius r₁ may be less than about 65% of r₂. In some aspects, radius r₁ may be about 59% of r₂. In some aspects, the radius r₁ may be less (e.g., smaller) than radius r₃. In some aspects, radius r₁ may be less than about 40% of r₃. In some aspects, radius r₁ may be less than about 45% of r₃. In some aspects, radius r₁ may be less than about 50% of r₃. In some aspects, radius r₁ may be about 41.7% of r₃.

The ring L₂ may include a radius r₂. In some aspects, radius 12 may be less than about 10.5 mm. In some aspects, radius r₂ may be less than about 10.75 mm. In some aspects, radius r₂ may be less than about 11.0 mm. In some aspects, radius r₂ may be about 10.73 mm. In some aspects, the radius r₂ may be less (e.g., smaller) than radius r₃. In some aspects, radius r₂ may be less than about 65% of r₃. In some aspects, radius r₂ may be less than about 70% of r₃. In some aspects, radius 12 may be less than about 75% of r₃. In some aspects, radius r₂ may be about 70.6% of r₃.

In some aspects, the sensor assembly 1400 (e.g., an outer perimeter of the sensor assembly 1400) may include a radius r₃. In some aspects, radius r₃ may be less than about 14.5 mm. In some aspects, radius r₃ may be less than about 15.0 mm. In some aspects, radius r₃ may be less than about 15.50 mm. In some aspects, radius r₃ may be less than about 16.0 mm. In some aspects, radius r₃ may be about 15.19 mm.

FIGS. 15B-15C illustrate an example sensor assembly 1500 and example light paths between emitters and detectors of the sensor assembly 1500.

FIG. 15B illustrates an example arrangement of emitter and detector chambers of the sensor assembly 1500. As shown, the sensor assembly 1500 can include a first emitter chamber 1536a enclosing a first emitter group comprising one or more emitters, a second emitter chamber 1536b enclosing a second emitter group comprising one or more emitters, one or more first detector chambers 1540, one or more second detector chambers 1542, and one or more third detector chambers 1538. In some aspects, each detector chamber may enclose one detector.

The first emitter group of the first emitter chamber 1536a may comprise the same number and type of emitters as the second emitter group of the second emitter chamber 1536b. In other words, each emitter of the first emitter group may correspond to an emitter of the same type (e.g., same wavelength) of the second emitter group. The emitters of the first emitter group may be arranged in a configuration that mirrors the emitters of the second emitter group across a centerline 1550 of the sensor assembly 1500 as shown in FIG. 15B. For example, each emitter of the first group of emitters may be located a distance away from a centerline 1550 of the sensor assembly 1500 that is a same distance that a corresponding emitter of the second group of emitters is located away from the centerline 1550 of the sensor assembly 1500. For example, the first and second emitter groups may each include an emitter that emits light of a first wavelength and that are positioned at locations that are mirror images of each other across a centerline 1550 of the sensor assembly 1500. Additionally, the first and second emitter groups may each include an emitter that emits light of a second wavelength and that are positioned at locations that are mirror images of each other across a centerline 1550 of the sensor assembly 1500. Each of the emitters of the first emitter group may correspond to an emitter of the second emitter group located at a mirror image position, and vice versa.

The one or more second detector chambers 1542 may be bisected by a centerline 1550 of the sensor assembly 1500. Each of the detectors of the respective one or more second detector chambers 1542 may be bisected by a centerline 1550 of the sensor assembly 1500. In other words, the one or more second detector chambers 1542 and the respective detectors and the sensor assembly 1500 may each share a same (e.g., parallel) centerline 1550. The sensor assembly 1500 may be oriented (e.g., rotated) with respect to the tissue of a wearer in any orientation. In an example implementation where the sensor assembly 1500 is worn on a wrist of a user, the sensor assembly 1500 may be rotated in any direction with respect to the wrist or forearm of the wearer. In one example configuration, the sensor assembly 1500 may be oriented with respect to the forearm (or other body part) of a wearer such that the centerline 1550 of the sensor assembly is perpendicular to a line extending along a length of the forearm of the wearer (e.g., from the elbow to the wrist). Advantageously, such a configuration may improve physiological measurements by facilitating light emitted from the emitter chambers and detected at the detector chambers (e.g., light travelling from emitter chamber 1536a to detector chamber 1538) to penetrate into soft tissue of the wearer (e.g., blood vessels) rather than other tissues such as bone. In another example configuration, the sensor assembly 1500 may be oriented with respect to the forearm (or other body part) of a wearer such that the centerline 1550 of the sensor assembly is parallel to a line extending along a length of the forearm of the wearer (e.g., from the elbow to the wrist). Advantageously, such a configuration may improve physiological measurements by facilitating light emitted from the emitter chambers and detected at the detector chambers (e.g., light travelling from emitter chamber 1536a to detector chamber 1542) to penetrate into soft tissue of the wearer (e.g., blood vessels) rather than other tissues such as bone.

As shown in FIG. 15B, emitters of the first and second emitter groups that correspond to each other (e.g., emit the same wavelength and mirror each other) may each emit light that travels along respective paths to the detectors of the one or more second detector chambers 1542. The respective paths of light from the corresponding emitters may be of equal length. This may be because the corresponding emitters are each positioned an equal distance away from a detector of a chamber 1542. The corresponding emitters may each be an equal distance away from a detector of a chamber 1542 because they are positioned at mirror images of each other across a centerline 1550 of the sensor assembly 1500 that bisects the one or more second detector chambers 1542 and respective detectors.

The one or more second detector chambers 1542 and their respective detectors may be used, at least in part, for calibration, for example to characterize the emitters, by providing known information such as a known ratio. For example, information corresponding to a wavelength detected at a detector of a chamber 1542 from an emitter of the first group of emitters may be similar or the same as information information corresponding to that wavelength detected at the detector of the chamber 1542 from an emitter of the second group of emitters and a comparison (e.g., subtracting, dividing, etc.) of the information resulting from the first and second groups of emitters may yield a known number such as zero or one because the corresponding emitters from the first and second emitter groups may be an equal distance from the detector of chamber 1542 and light emitted therefrom may travel a same distance to the detector of chamber 1542. As an example of normalization, ratios of wavelengths detected at detectors of chambers 1538, 1540 may be normalized (e.g., divided by) ratios of wavelengths detected at detectors of chambers 1542. In instances where the information resulting from detection of light from the first and second groups of emitters is not the same or is substantially different (e.g., as a result of emission intensity variations or other such discrepancies) the information may be adjusted or normalized (e.g., calibrated) to account for such differences. This normalization or on-board calibration or characterization of the emitters may improve accuracy of the physiological measurements and provide for continuous calibration or normalization during measurements. In some aspects, a processor may be configured to calibrate or normalize the physiological parameter measurement of the sensor continuously. In some aspects, a processor may be configured to calibrate or normalize the physiological parameter measurement of the sensor while the optical sensor measures physiological parameters of the wearer.

FIG. 15C illustrates an example arrangement of emitter and detector chambers of the sensor assembly 1500. As shown, the sensor assembly 1500 can include a first emitter chamber 1536a, a second emitter chamber 1536b, one or more first detector chambers 1540, one or more second detector chambers 1542, and one or more third detector chambers 1538, for example as discussed elsewhere herein.

The first and second emitter chambers 1536a, 1536b may be located at non-equal distances away from each of the chambers of the one or more detector chambers 1538, 1540. Thus, with respect to each detector chamber of the chambers 1538, 1540, the first and second emitter chamber 1536a, 1536b, may each be a “near” or “far” emitter chamber. In other words, each detector of the detector chambers 1538, 1540 may detect light, of any given wavelength, from both a “near” emitter and a “far” emitter, with the near and far emitters being included in either the first or second emitter group, respectively.

As an example, as shown in FIG. 15C, light of a given wavelength may travel along a path from an emitter in the first emitter group to the detector of detector chamber 1538 and light of the same wavelength may travel along a path from an emitter in the second emitter group to the same detector. The light from the first emitter group may travel along a longer path than light from the second emitter group before reaching the detector of chamber 1538. Thus, for any detector of detector chambers 1538 or 1540, the detector may receive light of a given wavelength from both a near (e.g., proximal) emitter and a far (e.g., distal) emitter. This may not be the case for detectors of chambers 1542 because the first and second emitter groups may each be located a same distance away from any given detector of detector chambers 1542, as described herein.

For convenience, the terms “proximal” and “distal” may be used herein to describe structures relative to any of the detector chambers or their respective detectors. For example, an emitter may be proximal to a detector chamber of the first detector chambers and distal to a detector of the second detector chambers. The term “distal” refers to one or more emitters that are farther away from a detector chamber than at least some of the other emitters. The term “proximal” refers to one or more emitters that are closer to a detector chamber than at least some of the other emitters. The term “proximal emitter” may be used interchangeably with “near emitter” and the term “distal emitter” may be used interchangeably with “far emitter”.

A single emitter may be both proximal to one detector and distal to another detector. For example, an emitter may be a proximal emitter relative to a detector of the first detector chambers and may be a distal emitter relative to a detector of the second detector chambers.

Light of a given wavelength that is detected at a detector may provide different information depending on the length of the path it has travelled from the emitter (e.g., along a long path from a distal emitter or along a short path from a proximal emitter). For example, light that has travelled along a long path from a distal emitter may penetrate deeper into the tissue of a wearer of the device and may provide information pertaining to pulsatile blood flow or constituents. The use of a proximal and distal emitter for each wavelength may improve accuracy of the measurement, for example information pertaining to light that has travelled along a long path from a distal emitter may be normalized by (e.g., divided by) information pertaining to light that has travelled along a short path from a proximal emitter.

FIGS. 15D-15G illustrate an example sensor assembly 1500 and example light barriers or light blocks between emitter and detector chambers of the sensor assembly 1500.

FIG. 15D is a front view of an example aspect of a sensor assembly 1500. The sensor assembly 1500 includes an opaque frame opaque frame 1526, one or more electrodes 1524, one or more detector chambers 1588, one or more emitter chambers 1578, and a light barrier construct 1520.

The opaque frame 1526 can include one or more materials configured to prevent or block the transmission of light. In some aspects, the opaque frame 1526 may form a single integrated unit. In some aspects, the opaque frame 1526 may be formed of a continuous material. The light barrier construct 1520 can include one or more materials configured to prevent or block the transmission of light. In some aspects, the light barrier construct 1520 may form a single integrated unit. In some aspects, the light barrier construct 1520 may be formed of a continuous material. In some aspects, the light barrier construct 1520 and the opaque frame 1526 may form a single integrated unit. In some aspects, the light barrier construct 1520 and the opaque frame 1526 may be separably connected.

The light barrier construct 1520 may include one or more light barriers, such as light barriers 1520a, 1520b, 1520c, 1520d, which are provided as non-limiting examples. In some aspects, light barriers may be also be referred to as light blocks herein. The light barriers may form one or more portions of the light barrier construct 1520. The light barrier construct 1520 (or light barrier portions thereof) may prevent light from passing therethrough. The light barrier construct 1520 may include spaces between various light barriers which may define one or more chambers (e.g., detector chambers 1588, emitter chambers 1578). In some aspects, the one or more chambers (e.g., detector chambers 1588, emitter chambers 1578) may be enclosed by the light barrier construct 1520 or light barrier portions thereof, a surface of a substrate (e.g., PCB), and a lens or cover. In some aspects, light may only enter the chambers through the lens or cover.

An example of a light barrier is provided with reference to example light barrier 1520a. Light barrier 1520a forms a portion of light barrier construct 1520. Light barrier 1520a may prevent (e.g., block) light from passing therethrough between adjacent chambers. For example, light barrier 1520a may prevent light from passing through the light barrier construct 1520 between an emitter chamber 1578 and a detector chamber 1588. Light barrier 1520a, or portions thereof, may include a width 1571. In some aspects, width 1571 may be less than about 3.30 mm. In some aspects, width 1571 may be less than about 3.25 mm. In some aspects, width 1571 may be less than about 3.20 mm. In some aspects, width 1571 may be about 3.24 mm. In some aspects, the width 1571 may be greater (e.g., larger) than length 1579. In some aspects, width 1571 may be less than about 165% of length 1579. In some aspects, width 1571 may be less than about 160% of length 1579. In some aspects, width 1571 may be less than about 155% of length 1579. In some aspects, width 1571 may be about 160% of length 1579. Advantageously, a greater width 1571 (e.g., a wider light barrier separating the emitter chambers 1578 and detector chambers 1588) may cause light emitted from the emitter chambers 1578 to travel a greater distance before reaching the detector chambers 1588. Light that travels a greater distance may penetrate deeper into the tissue of the wearer which may improve accuracy of a physiological measurement.

Another example of a light barrier is provided with reference to example light barrier 1520b. Light barrier 1520b forms a portion of light barrier construct 1520. Light barrier 1520b may prevent (e.g., block) light from passing therethrough between adjacent chambers. For example, light barrier 1520b may prevent light from passing through the light barrier construct 1520 between an emitter chamber 1578 and a detector chamber 1588. Light barrier 1520b, or portions thereof, may include a width 1572. In some aspects, width 1572 may be less than about 1.65 mm. In some aspects, width 1572 may be less than about 1.60 mm. In some aspects, width 1572 may be less than about 1.55 mm. In some aspects, width 1572 may be about 1.59 mm. In some aspects, the width 1572 may be less (e.g., smaller) than width 1571. In some aspects, width 1572 may be less than about 60% of width 1571. In some aspects, width 1572 may be less than about 55% of width 1571. In some aspects, width 1572 may be less than about 50% of width 1571. In some aspects, width 1572 may be about 49% of width 1571. Advantageously, a greater width 1572 may cause light emitted from the emitter chambers 1578 to travel a greater distance before reaching the detector chambers 1588. Light that travels a greater distance may penetrate deeper into the tissue of the wearer which may improve accuracy of a physiological measurement

Another example of a light barrier is provided with reference to example light barrier 1520c. Light barrier 1520c forms a portion of light barrier construct 1520. Light barrier 1520c may prevent (e.g., block) light from passing therethrough between adjacent chambers. For example, light barrier 1520c may prevent light from passing through the light barrier construct 1520 between adjacent detector chamber 1588.

Another example of a light barrier is provided with reference to example light barrier 1520d. Light barrier 1520d forms a portion of light barrier construct 1520. Light barrier 1520d may prevent (e.g., block) light from passing therethrough between adjacent chambers. For example, light barrier 1520d may prevent light from passing through the light barrier construct 1520 between adjacent emitter chambers 1578. In some aspects, light barrier 1520d may have a width 1575 separating adjacent emitter chambers of less than about 1.40 mm. In some aspects, width 1575 may be less than about 1.35 mm. In some aspects, width 1575 may be less than about 1.30 mm. In some aspects, width 1575 may be about 1.28 mm. In some aspects, width 1575 may be less (e.g., smaller) than width 1571. In some aspects, width 1575 may be less than about 50% of width 1571. In some aspects, width 1575 may be less than about 45% of width 1571. In some aspects, width 1575 may be less than about 40% of width 1571. In some aspects, width 1575 may be less than about 35% of width 1571. In some aspects, width 1575 may be about 39.5% of width 1571.

The emitter chambers 1578 are positioned within a central region of the sensor assembly 1500. The emitter chambers 1578 may be positioned adjacent to one another across a centerline of the sensor assembly 1500 as described in greater detail with reference to FIG. 15B, for example. The emitter chambers 1578 may be positioned adjacent the center point C′1. Each of the emitter chambers 1578 may be a similar size and/or shape. The emitter chambers 1578 may be separated, at least in part, by light barrier 1520d of the light barrier construct 1520. In some aspects, as shown in this example, the light barrier 1520d may form an entire distance between emitter chambers 1578. For example, emitter chambers 1578 may be separated by only the light barrier 1520d such that other components (e.g., detectors, detector chambers, etc.) are not positioned between the emitter chambers 1578.

A portion of the emitter chambers 1578 may extend a length 1579 away from center point C′1. In some aspects, length 1579 may be less than about 2.15 mm. In some aspects, length 1579 may be less than about 2.10 mm. In some aspects, length 1579 may be less than about 2.05 mm. In some aspects, length 1579 may be less than about 2.0 mm. In some aspects, length 1579 may be about 2.02 mm. In some aspects, the length 1579 may be less (e.g., smaller) than a width of a light barrier separating an emitter chamber from a detector chamber such as width 1571. In some aspects, length 1579 may be less than about 70% of width 1571. In some aspects, length 1579 may be less than about 65% of width 1571. In some aspects, length 1579 may be less than about 60% of width 1571. In some aspects, length 1579 may be about 62.3% of width 1571.

As shown in this example aspect, the detector chambers 1588 are arranged in a substantially circular pattern. Each of the detector chambers 1588 houses a detector 1506 positioned on a substrate (e.g., PCB) in a substantially circular or annular pattern. The detectors 1506 may be positioned in a central region of each of the respective detector chambers 1578. The detector chambers 1588 are arranged along a ring defined by ring L′1. In some aspects, such as shown in this example aspect, detectors 1506 of respective detector chambers 1588 may also be arranged along a same ring along which the detector chambers 1588 are arranged (such as in aspects where detectors are positioned in a central region of respective chambers). The ring L′₁ may intersect a central region of the detector chambers 1588. In this example aspect, the ring L′1 encloses an entirety of the emitter chambers emitter chambers 1578 such that the emitter chambers 1578 are positioned within an interior region (e.g., a central region) of the ring L′ defined by the detector chambers 1588. In some aspects, each of the detector chambers 1588 (and corresponding detectors 1506 within respective detector chambers 1588) may be positioned at a substantially similar or same distance away from the center point C′₁ (e.g., center of sensor assembly 1500). In some aspects, the detectors 1506 may be rectangular including longer sides and shorter sides. The detectors 1506 may be positioned on a substrate of the sensor assembly 1500 such that a long side of each detector is orthogonal to a radius extending away from center point C′₁ (e.g., radius r′₁, radius r′₂, radius r′₃). Advantageously, orienting the detectors 1506 on the sensor assembly 1500 in an annular arrangement with a long side of the detectors 1506 orthogonal the center point C′₁ may improve an accuracy of physiological measurements by ensuring that light from emitters travels along a known path length from emitters to the detectors 1506 and may also reduce processing requirements of the sensor assembly 1500 by reducing the amount of variables (e.g., number of light path lengths) required to process in order to determine physiological data.

The electrodes 1524 can include a reference electrode and a negative electrode (and/or a positive electrode). In some aspects, a wearable device such as a watch incorporating the sensor assembly 1500 can include another ECG electrode (e.g., a positive electrode) located on the housing of the wearable device configured to make contact with the wearer's skin. In some configurations, a surface of the electrodes 1524 may be flush with a surface of the opaque frame 1526.

The electrodes 1524 are positioned within or along a portion of the opaque frame 1526 such as shown in FIG. 14B for example. In some aspects, the electrodes 1524 can be substantially semicircular. In some aspects, the electrodes 1524 can be substantially semi-annular. In the example aspect shown, each of the electrodes 1524 forms a substantial half annulus. Advantageously, an annular shaped electrode may improve contact with the skin of a wearer (e.g., by contacting a diverse area of skin) while simultaneously reducing the amount of surface area of the electrode. In some aspects, each of the electrodes 1524 may be a similar size and/or shape. In some aspects, the electrodes 1524 may be various sizes and/or shapes. In this example aspect, the electrodes 1524 are positioned within the sensor assembly 1500 (e.g., within the opaque frame 1526) along ring defined by L′₂. In various aspects described herein, the ring L′₂ may include various radii which may advantageously provide improved contact between the electrodes 1524 and the skin of a wearer of the device. In some implementations, frame 1526 includes recesses 1525 that are sized and/or shaped to accommodate the ECG electrodes 1524. In some implementations, recesses 1525 have a depth (for example, measured from a plane of the frame 1526) that is substantially equal to a thickness of the ECG electrodes 1524. In some implementations, recesses 1525 have a size and/or shape that matches a size and/or shape of the ECG electrodes 1524. For example, in some implementations in which the ECG electrodes have a semi-annular shape, the recesses 1525 can have a semi-annular shape.

The opaque frame 1526 includes one or more gaps (e.g., g′₁, g′₂) between electrodes 1524. The gaps g′₁, g′₂, (or other portions of the opaque frame 1526) may electrically insulate each of the electrodes 1524 from one another. Each of the electrodes 1524 includes a curved edge along a portion of respective gaps g′₁, g′₂. In some aspects, the gaps g′₁, g′₂, may be a similar or a same size. In some aspects, the gaps g′₁, g′₂, may be a different size than each other. In some aspects, the gaps g′₁, g′₂, may be less than about 0.6 mm. In some aspects, the gaps g′₁, g′₂, may be less than about 0.65 mm. In some aspects, the gaps g′₁, g′₂, may be less than about 0.7 mm. In some aspects, the gaps g′₁, g′₂, may be about 0.62 mm. As discussed above, in some implementations the frame 1526 includes recesses 1525 sized and/or shaped to receive the ECG electrodes 1524. In some implementations, each of such recesses 1525 includes first and second ends, the first ends of the recesses 1525 are separated from one another by gap g′₁, and the second ends of the recesses 1525 are separated from one another by gap g′₂ (see FIG. 15D). In some implementations, such as that illustrated in at least FIG. 15D, ends of the recesses 1525 and/or ends of ECG electrodes 1524 have a rounded shape.

The ring L′₁ may be concentric with an outer perimeter of the sensor assembly 1500. The ring L′₂ may be concentric with an outer perimeter of the sensor assembly 1500. The ring L′₂ may be concentric with a ring defined by positions of the detector chambers 1588 such as ring L′₁. Center point C′₁ may define a geometric center of ring L′₁. Center point C′₁ may define a geometric center of ring L′₂. Center point C′₁ may define a geometric center of an outer perimeter of the sensor assembly 1500. In some aspects, such as shown in FIG. 15D, each of L′₁, L′₂, and an outer perimeter of the sensor assembly 1500 are concentric with each other and share a same geometric center shown as C′₁.

The ring L′₁ may include a radius r′₁. In some aspects, radius r′₁ may be less than about 6.5 mm. In some aspects, radius r′₁ may be less than about 6.45 mm. In some aspects, radius r′₁ may be less than about 6.40 mm. In some aspects, radius r′₁ may be about 6.40 mm. In some aspects, the radius r′₁ may be less (e.g., smaller) than radius r′₂. In some aspects, radius r′₁ may be less than about 60% of r′₂. In some aspects, radius r′₁ may be less than about 55% of r′₂. In some aspects, radius r′₁ may be less than about 50% of r′₂. In some aspects, radius r′₁ may be about 50.9% of r′₂. In some aspects, the radius r′₁ may be less (e.g., smaller) than radius r′₃. In some aspects, radius r′₁ may be less than about 40% of r′₃. In some aspects, radius r′₁ may be less than about 45% of r′₃. In some aspects, radius r′₁ may be less than about 50% of r′₃. In some aspects, radius r′₁ may be about 42% of r′₃.

The ring L′₂ may include a radius r′₂. In some aspects, radius r′₂ may be less than about 13 mm. In some aspects, radius r′₂ may be less than about 12.75 mm. In some aspects, radius r′₂ may be less than about 12.5 mm. In some aspects, radius r′₂ may be about 12.59 mm. In some aspects, the radius r′₂ may be less (e.g., smaller) than radius r′₃. In some aspects, radius r′₂ may be less than about 80% of r′₃. In some aspects, radius r′₂ may be less than about 85% of r′₃. In some aspects, radius r′₂ may be less than about 90% of r′₃. In some aspects, radius r′₂ may be about 82.7% of r′₃.

In some aspects, the sensor assembly 1500 (e.g., an outer perimeter of the sensor assembly 1500) may include a radius r′₃. In some aspects, radius r′₃ may be less than about 15 mm. In some aspects, radius r′₃ may be less than about 15.0 mm. In some aspects, radius r′₃ may be less than about 15.25 mm. In some aspects, radius r′₃ may be less than about 15.5 mm. In some aspects, radius r′₃ may be about 15.22 mm.

FIG. 15E is a side cutaway view of an example aspect of a sensor assembly 1500. The sensor assembly 1500 includes a barrier construct 1520, an outer surface 1591, and a substrate 1516. The outer surface 1591 may include light barrier construct portions, lens portions, opaque frame portions, and/or electrode portions. The outer surface 1591 of the sensor assembly 1500 may face and/or contact the skin of a wearer and may include a generally convex shape. A central region of the sensor assembly 1500 may have a height 1593. For example, the height of the light barrier construct 1520 at a central region of the sensor assembly 1500 may correspond to height 1593. The height 1593 may be a maximum distance the outer surface 1591 extends perpendicularly away from the substrate 1516 (e.g., toward the skin of a wearer). An outer region (e.g., along a perimeter of the substrate 1516) of the sensor assembly 1500 may have a height 1595. For example, the height of the light barrier construct 1520 and/or opaque frame 1526 at an outer region of the sensor assembly 1500 may correspond to height 1595. The height 1595 may be a minimum distance the outer surface 1591 extends perpendicularly away from the substrate 1516 (e.g., toward the skin of a wearer).

In some aspects, height 1593 may be between about 2.0 mm and about 3.0 mm, between about 2.5 mm and about 3.0 mm, between about 2.0 mm and about 2.5 mm, between about 1.5 mm and about 2.5 mm, between about 2.0 mm and about 2.25 mm, between about 2.25 mm and about 2.5 mm, or any value therebetween.

In some aspects, height 1595 may be less than about 1.40 mm. In some aspects, height 1595 may be less than about 1.35 mm. In some aspects, height 1595 may be less than about 1.30 mm. In some aspects, height 1595 may be less than about 1.25 mm. In some aspects, height 1595 may be about 1.29 mm. In some aspects, height 1595 may be less than about 1.90 mm. In some aspects, height 1595 may be less than about 1.85 mm. In some aspects, height 1595 may be less than about 1.80 mm. In some aspects, height 1595 may be less than about 1.75 mm. In some aspects, height 1595 may be about 1.78 mm.

In some aspects, the height 1593 may be greater (e.g., larger) than height 1595. In some aspects, height 1593 may be less than about 230% of height 1595. In some aspects, height 1593 may be less than about 225% of height 1595. In some aspects, height 1593 may be less than about 220% of height 1595. In some aspects, height 1593 may be less than about 215% of height 1595. In some aspects, height 1593 may be about 221% of height 1595. In some aspects, height 1593 may be less than about 155% of height 1595. In some aspects, height 1593 may be less than about 150% of height 1595. In some aspects, height 1593 may be less than about 145% of height 1595. In some aspects, height 1593 may be less than about 140% of height 1595. In some aspects, height 1593 may be about 145% of height 1595.

Advantageously, a greater height 1593 (and/or greater ratio of height 1593 to 1595) (for example, a taller light barrier at a central region of the sensor assembly 1400 may cause light emitted from emitter chambers to travel a greater distance before reaching the detector chambers. Light that travels a greater distance may penetrate deeper into the tissue of the wearer which may improve accuracy of a physiological measurement. A smaller height 1593 (and/or smaller ratio of height 1593 to 1595) may reduce discomfort to the wearer wearing the wearable device 10 or may reduce obstruction to blood flow of the wearer by reducing the amount of pressure the wearable device places on the wearer. The height 1593 and/or height 1595 may be selected to balance the above-mentioned considerations such as increasing the depth which light penetrates into the tissue and reducing discomfort or blood flow obstruction of the wearer.

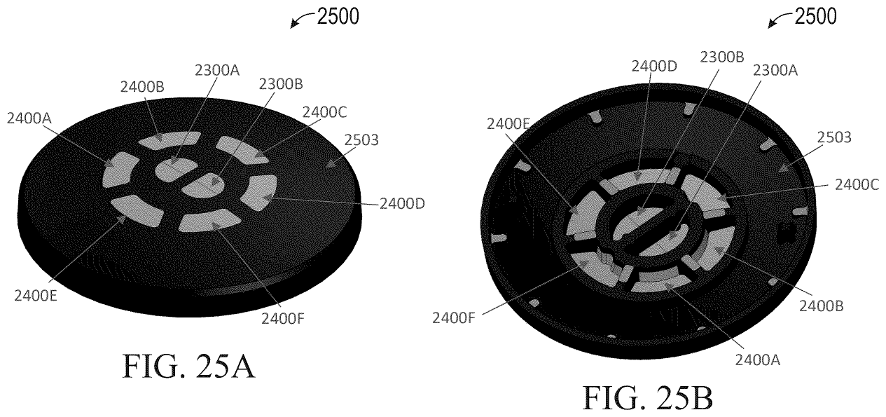

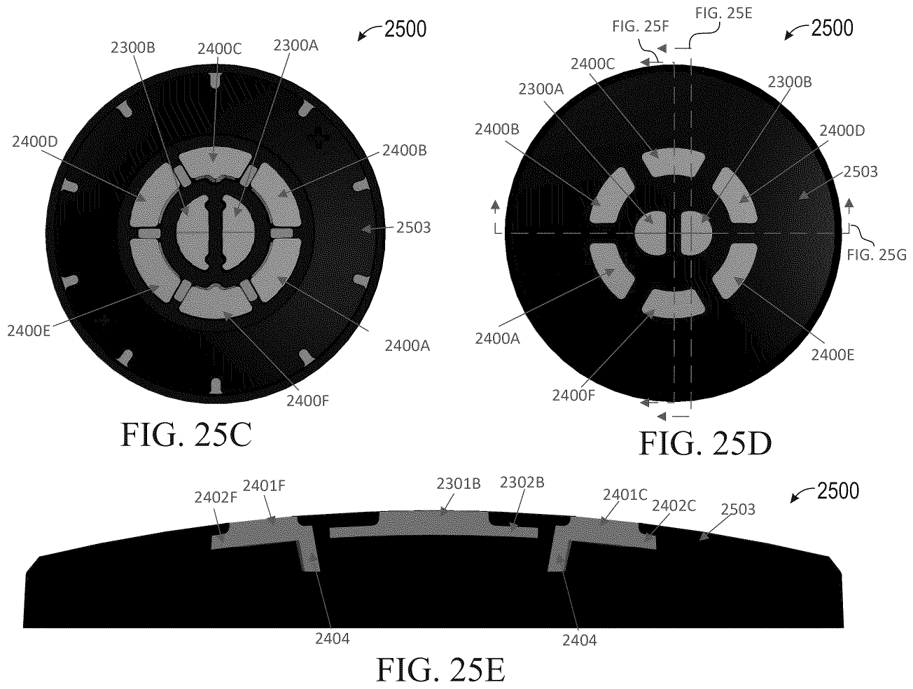

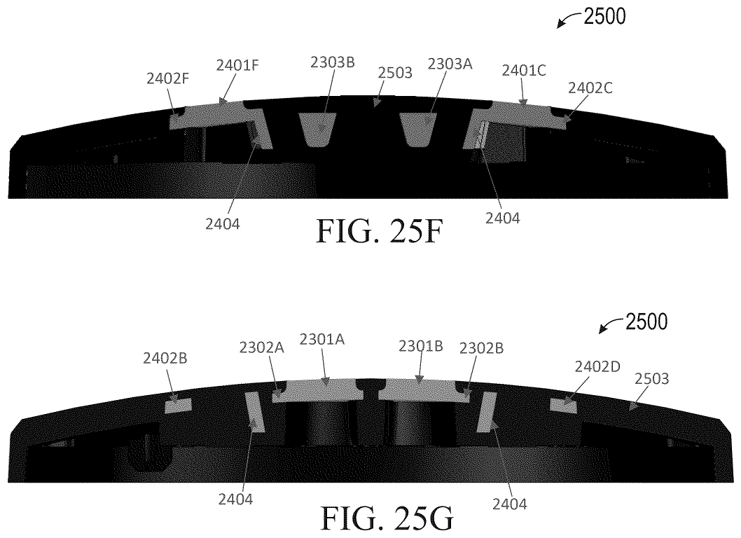

FIG. 15F and FIG. 15G illustrate two example aspects of a sensor assembly 1500 with different light barrier construct configurations. FIGS. 15F and 15G also show an example light path from an emitter chamber to a detector chamber. The light barrier construct 1520 (or portions thereof) shown in the example aspect of FIG. 15F may be taller (e.g., extending away from a surface of the substrate 1516) and/or wider than the light barrier construct 1520 (or portions thereof) shown in the example aspect of FIG. 15G. The greater height and/or width of the light barrier construct 1520 in the aspect of FIG. 25F may cause the light emitted from an emitter chamber 1578 to travel a greater distance before reaching a detector chamber and thus penetrate deeper into the tissue of the wearer than in the aspect of FIG. 15G. Thus, adjusting the height and/or width of the light barrier construct may affect the path the light travels from the emitter chamber to the detector chamber which may affect an accuracy of a physiological measurement. The height and/or width of the light barrier construct may be adjusted, according to various aspects, as required or desired.

FIG. 15H illustrates a cutaway side view of an example sensor assembly 1500 showing light transmissive lens(es) or cover(s) 1502 and light diffusing material. The light diffusing materials can be included in one or more of the emitter or detector chambers to improve distribution of emitted lighted and/or detected light. The diffusing materials or encapsulant, can include, for example, microspheres or glass microspheres. The encapsulant can eliminate air gaps between the surface of the light transmissive cover 1502 and the emitters and/or the detectors. The encapsulant can be included around the emitters to more evenly spread the emitted light, causing the emitted light to appear to be emitted from an entire emitter chamber rather than from a point source (that is, a single LED emitter) if the encapsulant were absent. The light transmissive lens(es) or cover(s) 1502 may include polycarbonate.

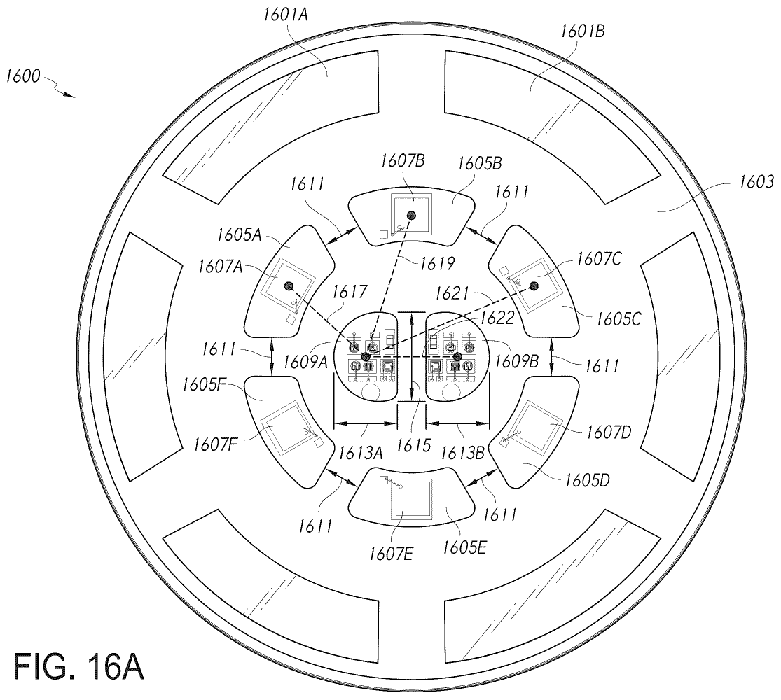

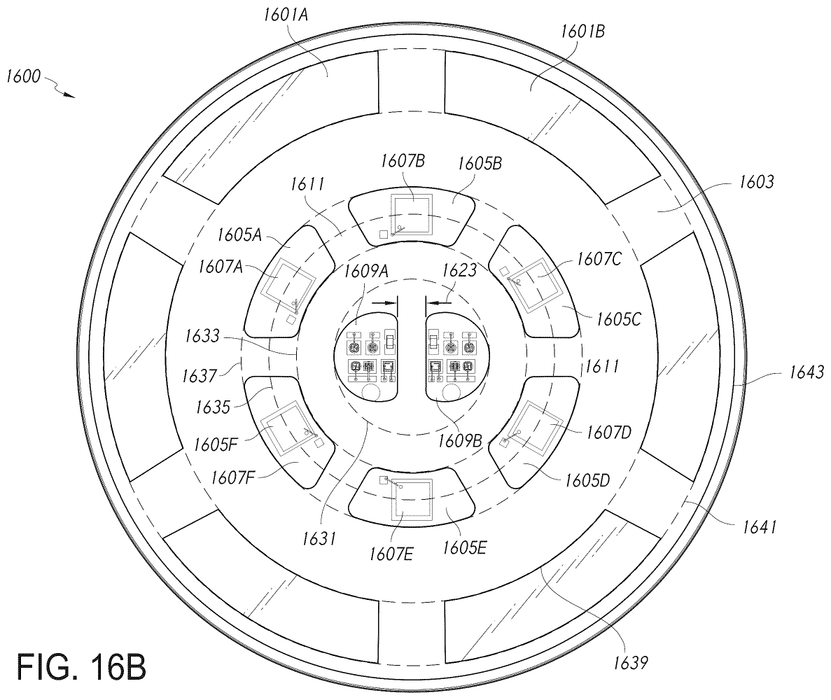

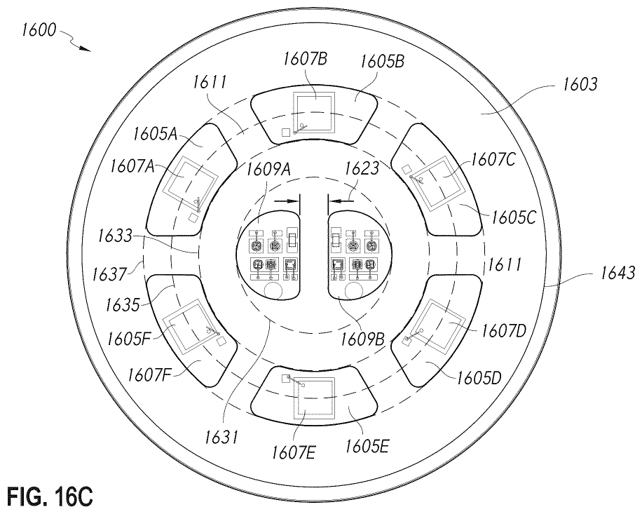

FIG. 16A is a front view of a tissue facing portion of a sensor assembly 1600. Sensor assembly 1600 can include any of the structural and/or operational features of any of the other example sensor assemblies shown and/or described herein. Sensor assembly 1600 can include a frame 1603, electrode 1601A, electrode 1601B, detector chambers 1605A-1605F, and emitter chambers 1609A, 1609B. Detector chambers 1605A-1605F can house one or more optical detectors. For example, each of detector chambers 1605A-1605F can house one of detectors 1607A-1607F, respectively. Emitter chambers 1609A, 1609B can house one or more optical emitters. For example, emitter chamber 1609A can house one, two, three, four, or five emitters and emitter chamber 1609B can house one, two, three, four, or five emitters that are separate from the emitters housed in emitter chamber 1609A. The emitters can include LEDs. Emitter chambers 1609A, 1609B can each respectively house a temperature sensor, such as a thermistor. Any of the example operational features, characteristics, structural features, including sizes, dimensions, proportions, etc. shown and/or described with reference to sensor assembly 1600 may apply to any of the other example sensor assemblies shown and/or described herein.

Detector chambers 1605A-1605F can be arranged within the frame 1603 in an annular formation, alternative oval, square, rectangular or other shaped arrangement can be used as well as would be understood by a person of skill in the art using disclosure herein as a guide. Detector chambers 1605A-1605F can surround the emitter chambers 1609A, 1609B. Emitter chambers 1609A, 1609B may be positioned at a central region of the sensor assembly 1600 and/or the frame 1603. Detector chambers 1605A-1605F may be spaced from one another by a distance 1611. For example, detector chamber 1605A may be spaced from detector chamber 1605F by a distance 1611. An edge of the detector chamber 1605A can be parallel to an edge of the detector chamber 1605F which edges may be separated by distance 1611. Distance 1611 can be greater than 1.0 mm. Distance 1611 can be less than 3.0 mm. Distance 1611 can be between about 0.2 mm and about 3.0 mm, between about 0.4 mm and about 2.8 mm, between about 0.6 mm and about 2.6 mm, between about 0.8 mm and about 2.4 mm, between about 1.0 mm and about 2.2 mm, between about 1.2 mm and about 2.0 mm, between about 1.4 mm and about 1.8 mm, between about 1.7 mm and about 1.8 mm, between about 1.5 mm and about 2.0 mm, or any value or range between any of these values or ranges or any value or range bounded by any combination of these values, although values or ranges outside these values or ranges can be used in some cases. In some implementations, distance 1611 can be about 1.767 mm. In some variants, detector chambers 1605A-1605F may be equally spaced from one another. For example, each of the distances 1611 may be the same or similar. In some variants, one or more of the distances 1611 shown between respective detector chambers may not be equal to one or more other distances 1611.

Emitter chambers 1609A, 1609B may have non-circular cross sections, as viewed from the front in FIG. 16A, but may have partially circular cross sections. Emitter chamber 1609A may have a width 1613A. Width 1613A can be greater than 2.0 mm. Width 1613A can be less than 5.0 mm. Width 1613A can be between about 2.0 mm and about 5.0 mm, between about 2.0 mm and about 4.0 mm, between about 2.2 mm and about 3.8 mm, between about 2.4 mm and about 3.6 mm, between about 2.6 mm and about 3.4 mm, between about 2.8 mm and about 3.2 mm, between about 2.75 mm and about 3.0 mm, between about 2.0 mm and about 3.0 mm, between about 2.5 mm and about 3.5 mm, between about 2.5 mm and about 3.0 mm, or any value or range between any of these values or ranges or any value or range bounded by any combination of these values, although values or ranges outside these values or ranges can be used in some cases. In some implementations, width 1613A may be about 2.878 mm. Width 1613A can be between about 50% and about 100% of a size of length 1615, between about 60% and about 90% of a size of length 1615, between about 60% and about 80% of a size of length 1615, between about 65% and about 75% of a size of length 1615, or any value or range between any of these values or ranges or any value or range bounded by any combination of these values, although values or ranges outside these values or ranges can be used in some cases. In some implementations, width 1613A can be about 71% of a size of length 1615. Emitter chamber 1609B can have a width 1613B which may be the same or similar as width 1613A. Emitter chamber 1609A may be a mirror image of emitter chamber 1609B across a centerline of the sensor assembly 1600 and/or the frame 1603.

Emitter chamber 1609A may have a length 1615. Length 1615 may be greater than width 1613A and/or width 1613B. Length 1615 can be greater than 3.0 mm. Length 1615 can be less than 6.0 mm. Length 1615 can be between about 3.0 mm and about 6.0 mm, between about 3.0 mm and about 5.0 mm, between about 3.2 mm and about 4.8 mm, between about 3.4 mm and about 4.6 mm, between about 3.6 mm and about 4.4 mm, between about 3.8 mm and about 4.2 mm, between about 3.0 mm and about 4.0 mm, between about 4.0 mm and about 5.0 mm, or any value or range between any of these values or ranges or any value or range bounded by any combination of these values, although values or ranges outside these values or ranges can be used in some cases. In some implementations, length 1615 may be about 4.046 mm. Emitter chamber 1609B can have a same length as emitter chamber 1609A.

Emitter chamber 1609A can be separated by emitter chamber 1609B by a distance 1622. The distance 1622 can extend from a geometric center of emitter chamber 1609A to a geometric center of emitter chamber 1609B. A center point of the distance 1622 may coincide with a geometric center of the sensor assembly 1600. Distance 1622 may be between about 2 mm and about 6 mm, between about 3 mm and about 5 mm, between about 3.25 mm and about 5 mm, between about 3.5 mm and about 5 mm, between about 3.75 mm and about 5 mm, between about 3 mm and about 4.75 mm, between about 3 mm and about 4.5 mm, between about 3 mm and about 4.25 mm, between about 3 mm and about 4 mm, between about 3.25 mm and about 4.25 mm, between about 3.5 mm and about 4.25 mm, between about 3.7 mm and about 4.25 mm, between about 3.7 mm and about 4.0 mm, or any value or range between any of these values or ranges or any value or range bounded by any combination of these values, although values or ranges outside these values or ranges can be used in some cases. In some implementations, distance 1622 may be about 3.792 mm.

Detector chamber 1605A and/or detector chamber 1607A can be separated from the emitter chamber 1609A by a distance 1617. Distance 1617 may extend from a geometric center of the emitter chamber 1609A to a geometric center of the detector chamber 1605A and/or to a geometric center of detector 1607A. A geometric center of the detector chamber 1605A may coincide with a geometric center of the detector 1607A, as may be the case for each of the detector chambers and their respective detectors. The geometric center of the emitter chamber 1609A may be positioned at a midline of the width 1613A and a midline of length 1615. The geometric center of the detector chamber 1605A and/or to a geometric center of detector 1607A may lie on a ring on which each of the other detector chambers and/or detectors lie, as shown and/or described in FIG. 16B, for example. Distance 1617 can be between about 3 mm and about 7 mm, between about 3 mm and about 6 mm, between about 3.2 mm and about 5.8 mm, between about 3.4 mm and about 5.6 mm, between about 3.6 mm and about 5.4 mm, between about 3.8 mm and about 5.2 mm, between about 4.0 mm and about 5.0 mm, between about 4.2 mm and about 4.8 mm, between about 4.4 mm and about 4.6 mm, between about 4.5 mm and about 4.7 mm, or any value or range between any of these values or ranges or any value or range bounded by any combination of these values, although values or ranges outside these values or ranges can be used in some cases. In some implementations, distance 1617 can be about 4.67 mm. Detector chamber 1605F and/or detector 1607F can be separated from the emitter chamber 1609A by a same distance as distance 1617. Detector chamber 1605C and/or detector 1607C can be separated from the emitter chamber 1609B by a same distance as distance 1617. Detector chamber 1605D and/or detector 1607D can be separated from the emitter chamber 1609B by a same distance as distance 1617.

Detector chamber 1605B and/or detector 1607B can be separated from the emitter chamber 1609A by a distance 1619. Distance 1619 may extend from a geometric center of the emitter chamber 1609A to a geometric center of the detector chamber 1605B and/or to a geometric center of detector 1607B. Distance 1619 may be between about 5.0 mm and about 8.0 mm, between about 5.2 mm and about 7.8 mm, between about 5.4 mm and about 7.6 mm, between about 5.6 mm and about 7.4 mm, between about 5.8 mm and about 7.2 mm, between about 6.0 mm and about 7.0 mm, between about 6.2 mm and about 6.8 mm, between about 6.5 mm and about 7.5 mm, between about 6.6 mm and about 7.2 mm, between about 6.7 mm and about 7.0 mm, or any value or range between any of these values or ranges or any value or range bounded by any combination of these values, although values or ranges outside these values or ranges can be used in some cases. In some implementations, distance 1619 may be about 6.83 mm. Detector chamber 1605B and/or detector 1607B can be separated from the emitter chamber 1609B by a same distance as distance 1619. Detector chamber 1605E and/or detector 1607E can be separated from the emitter chamber 1609A by a same distance as distance 1619. Detector chamber 1605E and/or detector 1607E can be separated from the emitter chamber 1609B by a same distance as distance 1619.

Detector chamber 1605C and/or detector 1607C can be separated from the emitter chamber 1609A by a distance 1621. Distance 1621 may extend from a geometric center of the emitter chamber 1609A to a geometric center of the detector chamber 1605C and/or to a geometric center of detector 1607C. Distance 1621 may be between about 7.0 mm and about 10 mm, between about 7.2 mm and about 9.8 mm, between about 7.4 mm and about 9.6 mm, between about 7.6 mm and about 9.4 mm, between about 7.8 mm and about 9.2 mm, between about 8.0 mm and about 9.0 mm, between about 8.2 mm and about 8.8 mm, between about 8.4 mm and about 8.6 mm, or any value or range between any of these values or ranges or any value or range bounded by any combination of these values, although values or ranges outside these values or ranges can be used in some cases. In some implementations, distance 1621 may be about 8.5 mm. Detector chamber 1605D and/or detector 1607D can be separated from the emitter chamber 1609A by a same distance as distance 1621. Detector chamber 1605A and/or detector 1607A can be separated from the emitter chamber 1609B by a same distance as distance 1621. Detector chamber 1605F and/or detector 1607F can be separated from the emitter chamber 1609B by a same distance as distance 1621.

Distance 1621 may be greater than distance 1619. Distance 1619 may be greater than distance 1617. Distance 1621 may be greater than distance 1617. Each of distances 1617, 1619, and 1621 may be different.

As shown and/or described, detector chamber 1605A and/or detector 1607A may be separated from emitter chamber 1609A by a different distance than emitter chamber 1609B. This may be the case for detector chambers 1605C, 1605D, and/or 1605F and/or for detectors 1607C, 1607D, and/or 1607F. Accordingly, optical radiation emanating from emitter chamber 1609A may travel a different (e.g., shorter) distance to arrive at detector chamber 1605A and/or detector 1607A than optical radiation emanating from emitter chamber 1609B. The depth to which optical radiation penetrates tissue may correspond to the distance it travels. For example, optical radiation travelling a longer distance may penetrate deeper into the tissue of a user than optical radiation travelling a shorter distance. In some implementations, depth of penetration may correspond linearly to distance travelled. In some implementations, depth of penetration may be twice a distance travelled. For example, light travelling 5 mm may penetrate tissue to a depth of 10 mm. The detectors can be modulated based on distance traveled by various optical radiation wavelengths. For example, detector 1607A can ignore optical radiation of a certain wavelength received from emitter chamber 1609A and can respond to optical radiation of that same wavelength when emitted from emitter chamber 1609B. For example, detector 1607A can respond to optical radiation having a green color (e.g., about 520 nm) when originating from emitter chamber 1609A and can ignore optical radiation at that same wavelength when originating from emitter chamber 1609B at least because optical radiation having a green color may not need to penetrate as deep into the tissue to provide accurate information and/or penetrating too deep may reduce information and/or accuracy. As another example, detector 1607A can respond to optical radiation having a red color or infrared radiation (e.g., between about 600 nm and about 1000 nm) when originating from emitter chamber 1609B and can ignore optical radiation at that same wavelength when originating from emitter chamber 1609A at least because optical radiation having a red color or infrared radiation that penetrates deeper into tissue may provide more information or more accurate information than if it does not penetrate as deep.

FIG. 16B is another front view of the sensor assembly 1600. Various rings 1631, 1633, 1635, 1637, 1639, and 1641 are shown as superimposed on the sensor assembly 1600. The rings 1631, 1633, 1635, 1637, 1639, and 1641 may be annular having respective radii and may be concentric with one another. The rings 1631, 1633, 1635, 1637, 1639, and 1641 may share a common center which may also coincide with a center of the sensor assembly 1600 and/or a center of the frame 1603.

Ring 1631 may encompass emitter chambers 1609A, 1609B. Ring 1631 may be tangential with at least a portion of an edge of emitter chamber 1609A and portion of an edge of emitter chamber 1609B. Ring 1631 may have a diameter between about 5.5 mm and about 8.5 mm, between about 5.7 mm and about 8.3 mm, between about 5.9 mm and about 8.1 mm, between about 6.1 mm and about 7.9 mm, between about 6.3 mm and about 7.7 mm, between about 6.5 mm and about 7.5 mm, between about 6.7 mm and about 7.3 mm, between about 6.9 mm and about 7.1 mm, or any value or range between any of these values or ranges or any value or range bounded by any combination of these values, although values or ranges outside these values or ranges can be used in some cases. Ring 1631 can have a diameter of at least 5 mm, at least 6 mm, at least 6.5 mm, at least 7 mm, at least 7.5 mm, at least 8 mm, or at least 8.5 mm. In some implementations, ring 1631 may have a diameter of about 7 mm.

Ring 1631 can have a surface area of πr², which in some cases may be about π3.5² mm=39 mm². Emitter chamber 1609A and emitter chamber 1609B may each have an optical radiation emitting area between about 8 mm² and about 11 mm², between about 8.2 mm² and about 10.8 mm², between about 8.4 mm² and about 10.6 mm², between about 8.6 mm² and about 10.4 mm², between about 8.8 mm² and about 10.2 mm², between about 9.0 mm² and about 10.0 mm², between about 9.2 mm² and about 9.8 mm², between about 9.4 mm² and about 9.7 mm², between about 9.55 mm² and about 9.75 mm², or any value or range between any of these values or ranges or any value or range bounded by any combination of these values, although values or ranges outside these values or ranges can be used in some cases. In some implementations, emitter chamber 1609A and emitter chamber 1609B may each have an area of about 9.67 mm². As used herein, the optical radiation emitting areas of the emitter chambers may refer to a surface area of a curved surface (e.g., a lens) covering the emitter chambers, and/or may refer to a cross sectional area of the emitter chambers, and/or may refer to a surface area of a portion of a PCB enclosed by the emitter chambers.

Emitter chamber 1609A and emitter chamber 1609B may have a total combined optical radiation emitting area between about 14 mm² and about 30 mm², between about 16 mm² and about 28 mm², between about 18 mm² and about 26 mm², between about 18 mm² and about 24 mm², between about 18 mm² and about 22 mm², between about 18 mm² and about 20 mm², between about 16 mm² and about 20 mm², or any value or range between any of these values or ranges or any value or range bounded by any combination of these values, although values or ranges outside these values or ranges can be used in some cases. In some implementations, emitter chamber 1609A and emitter chamber 1609B may have a total area of less than about 25 mm². In some implementations, emitter chamber 1609A and emitter chamber 1609B may have a total area of about 19.34 mm². Emitter chamber 1609A may have a same area as emitter chamber 1609B. The total optical radiation emitting area of emitter chamber 1609A and emitter chamber 1609B may be between about 25% and about 100% of the area of ring 1631, between about 25% and about 75% of the area of ring 1631, between about 35% and about 65% of the area of ring 1631, between about 40% and about 60% of the area of ring 1631, between about 45% and about 55% of the area of ring 1631, or any value or range between any of these values or ranges or any value or range bounded by any combination of these values, although values or ranges outside these values or ranges can be used in some cases. In some implementations, the total area of emitter chamber 1609A and emitter chamber 1609B may be about 50% of the area of ring 1631.

Emitter chamber 1609A can be separated from emitter chamber 1609B by a distance 1623. For example, an edge of emitter chamber 1609A can be parallel to an edge of emitter chamber 1069B which edges may be separated by distance 1623. Distance 1623 can be between about 0.2 mm and about 2.0 mm, between about 0.4 mm and about 1.8 mm, between about 0.6 mm and about 1.6 mm, between about 0.8 mm and about 1.4 mm, between about 0.9 mm and about 1.2 mm, between about 0.9 mm and about 1.1 mm, between about 0.8 mm and about 1.0 mm, or any value or range between any of these values or ranges or any value or range bounded by any combination of these values, although values or ranges outside these values or ranges can be used in some cases. In some implementations, distance 1623 may be about 0.914 mm.

Ring 1633 may be tangential with at least a portion of an edge of detector chambers 1605A-1605F, such as an inner edge. Ring 1633 may have a diameter between about 8 mm and about 13 mm, between about 8.2 mm and about 12.8 mm, between about 8.4 mm and about 12.6 mm, between about 8.6 mm and about 12.4 mm, between about 8.8 mm and about 12.2 mm, between about 9.0 mm and about 12.0 mm, between about 9.2 mm and about 11.8 mm, between about 9.4 mm and about 11.6 mm, between about 9.6 mm and about 11.4 mm, between about 9.8 mm and about 11.2 mm, between about 10.0 mm and about 11.0 mm, between about 10.2 mm and about 10.8 mm, between about 10.4 mm and about 10.6 mm, or any value or range between any of these values or ranges or any value or range bounded by any combination of these values, although values or ranges outside these values or ranges can be used in some cases. Ring 1633 can have a diameter of at least 8 mm, at least 9 mm, at least 10 mm, at least 10.5 mm, at least 11 mm, at least 11.5 mm, or at least 12 mm. In some implementations, ring 1633 may have a diameter of about 10.5 mm.

Ring 1635 may intersect detector chambers 1605A-1605F and/or detectors 1607A-1607F. In some implementations, ring 1635 may bisect a width of respective detector chamber 1605A-1605F. For example, a width of detector chambers 1605A-1605F extending between ring 1633 and ring 1637 may by bisected by ring 1635. Likewise, ring 1635 may bisect detectors 1607A-1607F. A geometric center of respective detector chambers 1605A-1605F and/or a geometric center of respective detectors 1607A-1607F may lie on ring 1635. Ring 1635 may have a diameter between about 10 mm and about 16 mm, between about 10 mm and about 15 mm, between about 10.5 mm and about 14.5 mm, between about 11 mm and about 14 mm, between about 11.5 mm and about 14.0 mm, between about 12.0 mm and about 14.0 mm, between about 12.5 mm and about 13.5 mm, or any value or range between any of these values or ranges or any value or range bounded by any combination of these values, although values or ranges outside these values or ranges can be used in some cases. Ring 1635 can have a diameter of at least 10 mm, at least 11 mm, at least 11.5 mm, at least 12 mm, at least 12.5 mm, at least 13 mm, at least 13.5 mm, at least 14 mm, at least 15 mm, at least 16, or at least 17 mm. In some implementations, ring 1635 may have a diameter of about 12.9 mm.

The size of ring 1635 (with detectors 1607A-1607F positioned thereon) may correspond to a distance that optical radiation travels from emitter chambers 1609A, 1609B to respective detectors. As discussed herein, depth that optical radiation penetrates into tissue may be proportional to the distance it travels. Moreover, more power may be required to drive an emitter as distance increases in order to generate a significant signal at a detector. For example, an emitter may need to emit optical radiation at greater intensity for the optical radiation to be detected at greater distances than at shorter distances. Accordingly, a larger ring 1635 may cause optical radiation to travel a greater distance from emitters to detectors resulting in deeper tissue penetration (which may lead to more accurate measurements) but requiring greater power consumption. And a smaller ring 1635 may cause optical radiation to travel a short distance from emitters to detectors resulting in shallower tissue penetration (which may lead to less accurate measurements) but requiring less power consumption. Accordingly, ring 1635 (with detectors positioned thereon) may be sized as large as reasonable to maximize tissue penetration and measurement accuracy while adhering to power consumption restraints. Accordingly, if power consumption were not a factor, it may be desirable for ring 1635 to be larger than shown and/or described in order to generate the best measurements. Moreover, if measurement accuracy were less of a concern, it may be desirable for ring 1635 to be smaller than shown and/or described in order to reduce power consumption.

Ring 1637 may be tangential with at least a portion of an edge of detector chambers 1605A-1605F, such as an outer edge. Ring 1637 may enclose detector chambers 1605A-1605F. Ring 1637 may have a diameter between about 12 mm and about 20 mm, between about 12 mm and about 19 mm, between about 12 mm and about 18 mm, between about 13 mm and about 17 mm, between about 14 mm and about 16 mm, between about 14.5 mm and about 15.5 mm, between about 15 mm and about 16 mm, between about 15.25 mm and about 15.75 mm, or any value or range between any of these values or ranges or any value or range bounded by any combination of these values, although values or ranges outside these values or ranges can be used in some cases. Ring 1637 can have a diameter of at least 13 mm, at least 14 mm, at least 15 mm, at least 16 mm, at least 17 mm, at least 18 mm, or at least 19 mm. In some implementations, ring 1637 may have a diameter of about 15.4 mm.

A distance between ring 1637 and ring 1633 may correspond to a width of detector chambers 1605A-1605F. The distance between ring 1637 and ring 1633 may be a difference between any of the example radii of ring 1637 and ring 1633 shown and/or described herein. For example, the distance between ring 1637 and ring 1633 may be between about 1.5 mm and about 3.5 mm, between about 1.7 mm and about 3.3 mm, between about 1.9 mm and about 3.1 mm, between about 2.1 mm and about 2.9 mm, between about 2.3 mm and about 2.7 mm, between about 2.4 mm and about 2.6 mm, or any value or range between any of these values or ranges or any value or range bounded by any combination of these values, although values or ranges outside these values or ranges can be used in some cases. In some implementations, the distance between ring 1637 and ring 1633 may be about 2.45 mm.

Ring 1639 may be tangential with at least a portion of an edge of electrodes 1601A, 1601B, such as an inner edge. Ring 1639 may have a diameter between about 15 mm and about 29 mm, between about 16 mm and about 28 mm, between about 17 mm and about 27 mm, between about 18 mm and about 26 mm, between about 19 mm and about 25 mm, between about 20 mm and about 24 mm, between about 21 mm and about 23 mm, between about 22 mm and about 23 mm, or any value or range between any of these values or ranges or any value or range bounded by any combination of these values, although values or ranges outside these values or ranges can be used in some cases. In some implementations, ring 1639 may have a diameter of about 22.3 mm.

Ring 1641 may be tangential with at least a portion of an edge of electrodes 1601A, 1601B, such as an outer edge. Ring 1641 may enclose electrodes 1601A, 1601B. Ring 1641 may have a diameter between about 23 mm and about 36 mm, between about 23 mm and about 33 mm, between about 24 mm and about 32 mm, between about 25 mm and about 31 mm, between about 26 mm and about 29 mm, between about 27 mm and about 29 mm, or any value or range between any of these values or ranges or any value or range bounded by any combination of these values, although values or ranges outside these values or ranges can be used in some cases. In some implementations, ring 1641 may have a diameter of about 27.9 mm.

A distance between ring 1641 and ring 1639 may correspond to a width of electrodes 1601A, 1601B. The distance between ring 1641 and ring 1639 may be a difference between any of the example radii of ring 1641 and ring 1639 shown and/or described herein. For example, the distance between ring 1641 and ring 1639 may be between about 1.8 mm and about 4.8 mm, between about 1.8 mm and about 3.8 mm, between about 2.0 mm and about 3.6 mm, between about 2.2 mm and about 3.4 mm, between about 2.4 mm and about 3.2 mm, between about 2.6 mm and about 3.0 mm, between about 2.7 mm and about 2.9 mm, or any value or range between any of these values or ranges or any value or range bounded by any combination of these values, although values or ranges outside these values or ranges can be used in some cases. In some implementations, the distance between ring 1637 and ring 1633 may be about 2.8 mm.

Perimeter 1643 may bound a portion of the sensor assembly 1600 that is configured to contact tissue of a user when worn by the user. The portion of the sensor assembly 1600 that is bounded by perimeter 1643 may be curved, for example convex or non-planar. Perimeter 1643 may circular. Perimeter 1643 may have a diameter between about 20 mm and about 40 mm, between about 22 mm and about 38 mm, between about 24 mm and about 36 mm, between about 26 mm and about 34 mm, between about 28 mm and about 32 mm, between about 28 mm and about 30 mm, between about 25 mm and about 35 mm, or any value or range between any of these values or ranges or any value or range bounded by any combination of these values, although values or ranges outside these values or ranges can be used in some cases. In some implementations, perimeter 1643 may have a diameter of about 29.3 mm. Perimeter 1643 can define a surface area of πr², which in some cases may be about π14.64² mm=673.3 mm².

The surface area of the portion of the sensor assembly 1600 configured to contact the tissue of a wearer (e.g., a surface of the sensor assembly 1600 bounded by perimeter 1643) may be between about 600 mm² and about 800 mm², between about 625 mm² and about 775 mm², between about 625 mm² and about 750 mm², between about 650 mm² and about 725 mm², between about 650 mm² and about 700 mm², between about 660 mm² and about 680 mm², between about 600 mm² and about 700 mm², or any value or range between any of these values or ranges or any value or range bounded by any combination of these values, although values or ranges outside these values or ranges can be used in some cases.

Any of detector chambers 1605A-1605F may have an optical radiation detecting area between about 5 mm² and about 50 mm², between about 7 mm² and about 40 mm², between about 9 mm² and about 30 mm², between about 10 mm² and about 20 mm², between about 10 mm² and about 16 mm², between about 10 mm² and about 14 mm², between about 10.25 mm² and about 14 mm², between about 10.5 mm² and about 14 mm², between about 10.75 mm² and about 14 mm², between about 11 mm² and about 14 mm², between about 11.25 mm² and about 14 mm², between about 11.5 mm² and about 14 mm², between about 11.75 mm² and about 14 mm², between about 12 mm² and about 14 mm², between about 10 mm² and about 13.75 mm², between about 10 mm² and about 13.5 mm², between about 10 mm² and about 13.25 mm², between about 10 mm² and about 13 mm², between about 10 mm² and about 12.75 mm², between about 10 mm² and about 12.5 mm², between about 11 mm² and about 13 mm², between about 11.5 mm² and about 12.75 mm², between about 12 mm² and about 12.5 mm², or any value or range between any of these values or ranges or any value or range bounded by any combination of these values, although values or ranges outside these values or ranges can be used in some cases. In some implementations, any of detector chambers 1605A-1605F may have an area of about 12.258 mm². One or more of the detector chambers 1605A-1605F may have a same area as each other. As used herein, the optical radiation detecting areas of the detector chambers may refer to a surface area of a curved surface (e.g., a lens) covering the detector chambers, and/or may refer to a cross sectional area of the detector chambers, and/or may refer to a surface area of a portion of a PCB enclosed by the detector chambers.

Detector chambers 1605A-1605F may have a total combined optical radiation detecting area between about 50 mm² and about 100 mm², between about 60 mm² and about 100 mm², between about 70 mm² and about 100 mm², between about 80 mm² and about 100 mm², between about 50 mm² and about 90 mm², between about 50 mm² and about 80 mm², between about 50 mm² and about 70 mm², between about 60 mm² and about 90 mm², between about 70 mm² and about 80 mm², between about 70 mm² and about 75 mm², or any value or range between any of these values or ranges or any value or range bounded by any combination of these values, although values or ranges outside these values or ranges can be used in some cases. In some implementations, detector chambers 1605A-1605F may have a total area of about 73.548 mm².

Any of the detector chambers 1605A-1605F may have an optical radiation detecting area that is greater than an optical radiation emitting area of any of the emitter chambers 1609A-1609B. Any of the detector chambers 1605A-1605F may have an optical radiation detecting area that is between about 100% and about 150%, between about 100% and about 140%, between about 100% and about 130%, between about 110% and about 150%, between about 120% and about 150%, between about 130% and about 150%, between about 110% and about 140%, or between about 120% and about 130% of the optical radiation emitting area of any of the emitter chambers 1609A-1609B, or any value or range between any of these values or ranges or any value or range bounded by any combination of these values, although values or ranges outside these values or ranges can be used in some cases. In some implementations, any of the detector chambers 1605A-1605F may have an optical radiation detecting area that is about 126.6% of the optical radiation emitting area of any of the emitter chambers 1609A-1609B.

Any of the emitter chambers 1609A-1609B may have an optical radiation emitting area that is between about 5% and about 20%, between about 5% and about 18%, between about 5% and about 16%, between about 5% and about 14%, between about 8% and about 20%, between about 10% and about 20%, between about 12% and about 20%, between about 10% and about 15%, or between about 11% and about 14% of the total optical radiation detecting area of the detector chambers 1605A-1605F, or any value or range between any of these values or ranges or any value or range bounded by any combination of these values, although values or ranges outside these values or ranges can be used in some cases. In some implementations, any of the emitter chambers 1609A-1609B may have an optical radiation emitting area that is about 13.2% of the total optical radiation detecting area of the detector chambers 1605A-1605F.

Detector chambers 1605A-1605F may have a total combined optical radiation detecting area between about 5% and about 15%, between about 5% and about 13%, between about 5% and about 11%, between about 7% and about 15%, between about 9% and about 15%, between about 11% and about 15%, between about 7% and about 13%, between about 9% and about 12%, between about 10% and about 12%, between about 10.5% and about 11.5% of a surface area of the sensor assembly 1600 bounded by perimeter 1643, or any value or range between any of these values or ranges or any value or range bounded by any combination of these values, although values or ranges outside these values or ranges can be used in some cases. In some implementations, detector chambers 1605A-1605F may have a total combined optical radiation detecting area of about 10.9% of a surface area of the sensor assembly 1600 bounded by perimeter 1643.

Emitter chambers 1609A-1609B may have a total combined optical radiation emitting area between about 1% and about 5%, between about 1.2% and about 4.8%, between about 1.4% and about 4.6%, between about 1.6% and about 4.4%, between about 1.8% and about 4.2%, between about 2% and about 4%, between about 2.2% and about 3.8%, between about 2.4% and about 3.6%, between about 2.6% and about 3.4%, between about 2.8% and about 3.2%, between about 2.8% and about 3.0% of a surface area of the sensor assembly 1600 bounded by perimeter 1643, or any value or range between any of these values or ranges or any value or range bounded by any combination of these values, although values or ranges outside these values or ranges can be used in some cases. In some implementations, emitter chambers 1609A-1609B may have a total combined optical radiation emitting area of about 2.9% of a surface area of the sensor assembly 1600 bounded by perimeter 1643.

FIG. 16C is a front view of another example implementation of sensor assembly 1600 without electrodes. In this example, perimeter 1643 is smaller than in FIGS. 16A-16B and can have a diameter between about 15 mm and about 25 mm, between about 17 mm and about 25 mm, between about 19 mm and about 21 mm, between about 23 mm and about 25 mm, between about 15 mm and about 23 mm, between about 15 mm and about 21 mm, between about 15 mm and about 19 mm, between about 17 mm and about 23 mm, between about 19 mm and about 21 mm, between about 20 mm and about 22 mm, or any value or range between any of these values or ranges or any value or range bounded by any combination of these values, although values or ranges outside these values or ranges can be used in some cases. In some implementations, perimeter 1643 can have a diameter of about 20.9 mm. Perimeter 1643 can define a surface area of πr², which in some cases may be about π10.452 mm=343.1 mm².

The surface area of the portion of the sensor assembly 1600 configured to contact the tissue of a wearer (e.g., a surface of the sensor assembly 1600 bounded by perimeter 1643) may be between about 300 mm² and about 400 mm², between about 325 mm² and about 400 mm², between about 350 mm² and about 400 mm², between about 375 mm² and about 400 mm², between about 300 mm² and about 375 mm², between about 300 mm² and about 350 mm², between about 300 mm² and about 325 mm², between about 325 mm² and about 375 mm², between about 325 mm² and about 350 mm², or any value or range between any of these values or ranges or any value or range bounded by any combination of these values, although values or ranges outside these values or ranges can be used in some cases.

Detector chambers 1605A-1605F may have a total combined optical radiation detecting area between about 10% and about 40%, between about 10% and about 30%, between about 10% and about 25%, between about 10% and about 20%, between about 15% and about 30%, between about 15% and about 25%, between about 15% and about 25%, between about 15% and about 20%, between about 20% and about 40%, between about 20% and about 30%, between about 20% and about 25% of a surface area of the sensor assembly 1600 bounded by perimeter 1643, or any value or range between any of these values or ranges or any value or range bounded by any combination of these values, although values or ranges outside these values or ranges can be used in some cases. In some implementations, detector chambers 1605A-1605F may have a total combined optical radiation detecting area of about 21% of a surface area of the sensor assembly 1600 bounded by perimeter 1643.

Emitter chambers 1609A-1609B may have a total combined optical radiation emitting area between about 1% and about 10%, between about 1% and about 9%, between about 1% and about 8%, between about 1% and about 7%, between about 1% and about 6%, between about 1% and about 5%, between about 2% and about 9%, between about 2% and about 7%, between about 3% and about 9%, between about 3% and about 7%, between about 4% and about 9%, between about 4% and about 7%, between about 5% and about 9%, between about 5% and about 7%, between about 5% and about 6% of a surface area of the sensor assembly 1600 bounded by perimeter 1643, or any value or range between any of these values or ranges or any value or range bounded by any combination of these values, although values or ranges outside these values or ranges can be used in some cases. In some implementations, emitter chambers 1609A-1609B may have a total combined optical radiation emitting area of about 5.6% of a surface area of the sensor assembly 1600 bounded by perimeter 1643.

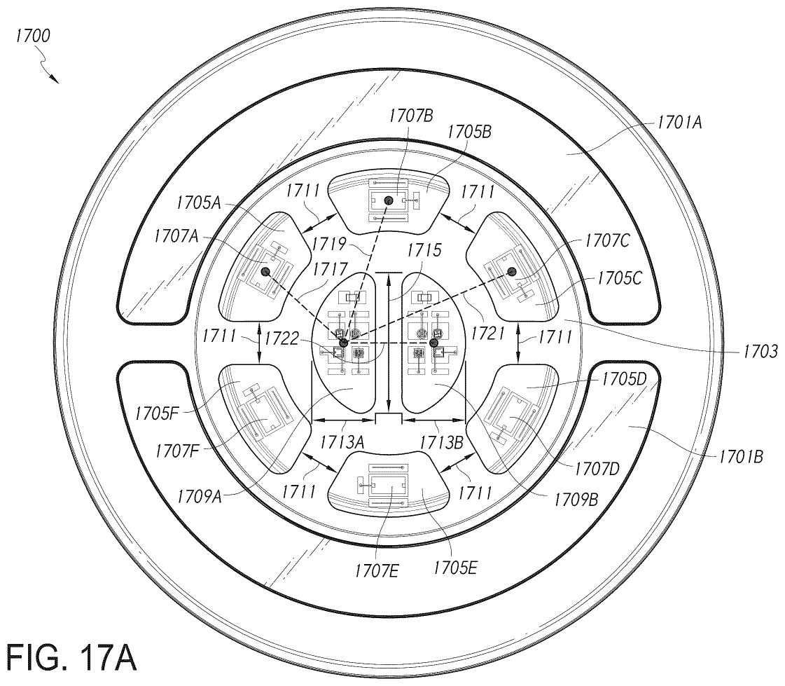

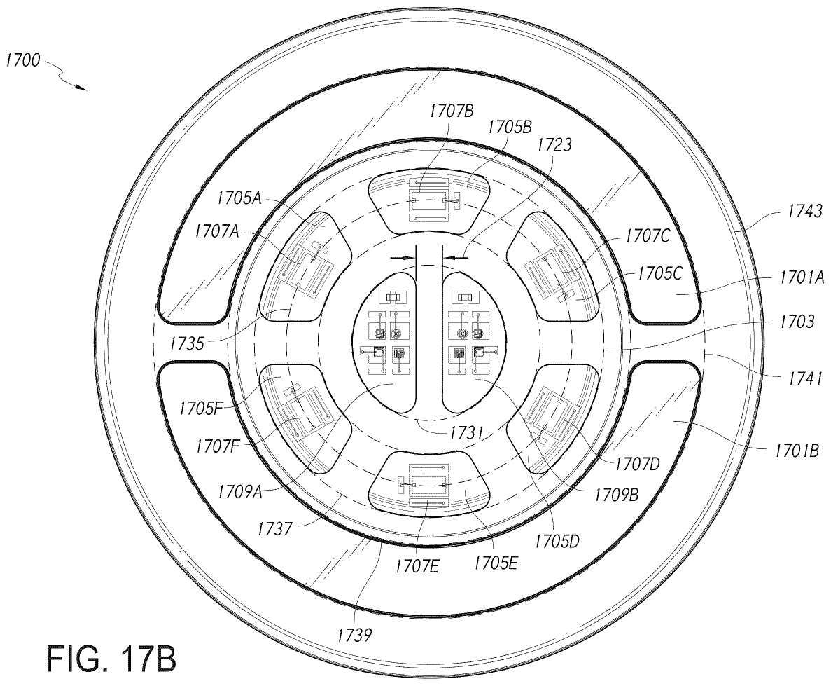

FIG. 17A is a front view of a tissue facing portion of a sensor assembly 1700. Sensor assembly 1700 can include any of the structural and/or operational features of any of the other example sensor assemblies shown and/or described herein. Sensor assembly 1700 can include a frame 1703, electrode 1701A, electrode 1701B, detector chambers 1705A-1705F, and emitter chambers 1709A, 1709B. Detector chambers 1705A-1705F can house one or more optical detectors. For example, each of detector chambers 1705A-1705F can house one of detectors 1707A-1707F, respectively. Emitter chambers 1709A, 1709B can house one or more optical emitters. For example, emitter chamber 1709A can house one, two, three, four, or five emitters and emitter chamber 1709B can house one, two, three, four, or five emitters that are separate from the emitters housed in emitter chamber 1709A. The emitters can include LEDs. Emitter chambers 1709A, 1709B can each respectively house a temperature sensor, such as a thermistor. Any of the example operational features, characteristics, structural features, including sizes, dimensions, proportions, etc. shown and/or described with reference to sensor assembly 1700 may apply to any of the other example sensor assemblies shown and/or described herein.

Detector chambers 1705A-1705F can be arranged within the frame 1703 in an annular formation. Detector chambers 1705A-1705F can surround the emitter chambers 1709A, 1709B. Emitter chambers 1709A, 1709B may be positioned at a central region of the sensor assembly 1700 and/or the frame 1703. Detector chambers 1705A-1705F may be spaced from one another by a distance 1711. For example, detector chamber 1705A may be spaced from detector chamber 1705F by a distance 1711. An edge of the detector chamber 1705A can be parallel to an edge of the detector chamber 1705F which edges may be separated by distance 1711. Distance 1711 can be greater than 1.0 mm. Distance 1711 can be less than 3.0 mm. Distance 1711 can be between about 0.2 mm and about 3.0 mm, between about 0.4 mm and about 3.0 mm, between about 0.6 mm and about 3.0 mm, between about 0.8 mm and about 3.0 mm, between about 1.0 mm and about 3.0 mm, between about 1.2 mm and about 2.8 mm, between about 1.4 mm and about 2.6 mm, between about 1.6 mm and about 2.4 mm, between about 1.8 mm and about 2.2 mm, between about 1.9 mm and about 2.1 mm, or any value or range between any of these values or ranges or any value or range bounded by any combination of these values, although values or ranges outside these values or ranges can be used in some cases. In some implementations, distance 1711 can be about 1.956 mm. In some variants, detector chambers 1705A-1705F may be equally spaced from one another. For example, each of the distances 1711 may be the same or similar. In some variants, one or more of the distances 1711 shown between respective detector chambers may not be equal to one or more other distances 1711.

Emitter chambers 1709A, 1709B may have non-circular cross sections, as viewed from the front in FIG. 17A, but may have partially circular cross sections. Emitter chamber 1709A may have a width 1713A. Width 1713A can be greater than 2.0 mm. Width 1713A can be less than 5.0 mm. Width 1713A can be between about 2.0 mm and about 5.0 mm, between about 2.0 mm and about 4.0 mm, between about 2.2 mm and about 3.8 mm, between about 2.4 mm and about 3.6 mm, between about 2.6 mm and about 3.4 mm, between about 2.8 mm and about 3.2 mm, between about 2.8 mm and about 3.0 mm, or any value or range between any of these values or ranges or any value or range bounded by any combination of these values, although values or ranges outside these values or ranges can be used in some cases. In some implementations, width 1713A may be about 2.886 mm. Width 1713A can be between about 25% and about 100% of a size of length 1715, between about 25% and about 75% of a size of length 1715, between about 30% and about 65% of a size of length 1715, between about 35% and about 55% of a size of length 1715, between about 40% and about 50% of a size of length 1715, or any value or range between any of these values or ranges or any value or range bounded by any combination of these values, although values or ranges outside these values or ranges can be used in some cases. In some implementations, width 1713A can be about 46.5% of a size of length 1715. Emitter chamber 1709B can have a width 1713B which may be the same or similar as width 1713A. Emitter chamber 1709A may be a mirror image of emitter chamber 1709B across a centerline of the sensor assembly 1700 and/or the frame 1703.

Emitter chamber 1709A may have a length 1715. Length 1715 may be greater than width 1713A and/or width 1713B. Length 1715 can be greater than 4.0 mm. Length 1715 can be less than 8.0 mm. Length 1715 can be between about 4.0 mm and about 8.0 mm, between about 4.2 mm and about 7.8 mm, between about 4.4 mm and about 7.6 mm, between about 4.6 mm and about 7.4 mm, between about 4.8 mm and about 7.2 mm, between about 5.0 mm and about 7.0 mm, between about 5.2 mm and about 6.8 mm, between about 5.4 mm and about 6.6 mm, between about 5.6 mm and about 6.4 mm, between about 5.8 mm and about 6.4 mm, between about 6.0 mm and about 6.4 mm, or any value or range between any of these values or ranges or any value or range bounded by any combination of these values, although values or ranges outside these values or ranges can be used in some cases. In some implementations, length 1715 may be about 6.2 mm. Emitter chamber 1709B can have a same length as emitter chamber 1709A.

Emitter chamber 1709A can be separated by emitter chamber 1709B by a distance 1722. The distance 1722 can extend from a geometric center of emitter chamber 1709A to a geometric center of emitter chamber 1709B. A center point of the distance 1722 may coincide with a geometric center of the sensor assembly 1700. Distance 1722 may be between about 2 mm and about 6 mm, between about 3 mm and about 5 mm, between about 3.25 mm and about 5 mm, between about 3.5 mm and about 5 mm, between about 3.75 mm and about 5 mm, between about 3 mm and about 4.75 mm, between about 3 mm and about 4.5 mm, between about 3 mm and about 4.25 mm, between about 3 mm and about 4 mm, between about 3.25 mm and about 4.25 mm, between about 3.2 mm and about 4.8 mm, between about 3.4 mm and about 4.6 mm, between about 3.6 mm and about 4.4 mm, between about 3.8 mm and about 4.2 mm, or any value or range between any of these values or ranges or any value or range bounded by any combination of these values, although values or ranges outside these values or ranges can be used in some cases. In some implementations, distance 1722 may be about 4.09 mm.

Detector chamber 1705A and/or detector 1707A can be separated from the emitter chamber 1709A by a distance 1717. Distance 1717 may extend from a geometric center of the emitter chamber 1709A to a geometric center of the detector chamber 1705A and/or to a geometric center of detector 1707A. A geometric center of the detector chamber 1705A may coincide with a geometric center of the detector 1707A, as may be the case for each of the detector chambers and their respective detectors. The geometric center of the emitter chamber 1709A may be positioned at a midline of the width 1713A and a midline of length 1715. The geometric center of the detector chamber 1705A and/or to a geometric center of detector 1707A may lie on a ring on which each of the other detector chambers and/or detectors lie, as shown and/or described in FIG. 17B, for example. Distance 1717 can be between about 3 mm and about 7 mm, between about 4 mm and about 6 mm, between about 4.0 mm and about 6.0 mm, between about 4.2 mm and about 5.8 mm, between about 4.4 mm and about 5.6 mm, between about 4.6 mm and about 5.4 mm, between about 4.8 mm and about 5.2 mm, between about 4.8 mm and about 5.0 mm, or any value or range between any of these values or ranges or any value or range bounded by any combination of these values, although values or ranges outside these values or ranges can be used in some cases. In some implementations, distance 1717 can be about 4.9 mm. Detector chamber 1705F and/or detector 1707F can be separated from the emitter chamber 1709A by a same distance as distance 1717. Detector chamber 1705C and/or detector 1707C can be separated from the emitter chamber 1709B by a same distance as distance 1717. Detector chamber 1705D and/or detector 1707D can be separated from the emitter chamber 1709B by a same distance as distance 1717.

Detector chamber 1705B and/or detector 1707B can be separated from the emitter chamber 1709A by a distance 1719. Distance 1719 may extend from a geometric center of the emitter chamber 1709A to a geometric center of the detector chamber 1705B and/or to a geometric center of detector 1707B. Distance 1719 may be between about 5.0 mm and about 8.0 mm, between about 5.2 mm and about 7.8 mm, between about 5.4 mm and about 7.6 mm, between about 5.6 mm and about 7.4 mm, between about 5.8 mm and about 7.2 mm, between about 6.0 mm and about 7.0 mm, between about 6.2 mm and about 6.8 mm, between about 6.5 mm and about 7.5 mm, between about 6.6 mm and about 7.2 mm, between about 6.6 mm and about 7.0 mm, between about 6.6 mm and about 6.8 mm, or any value or range between any of these values or ranges or any value or range bounded by any combination of these values, although values or ranges outside these values or ranges can be used in some cases. In some implementations, distance 1719 may be about 6.73 mm. Detector chamber 1705B and/or detector 1707B can be separated from the emitter chamber 1709B by a same distance as distance 1719. Detector chamber 1705E and/or detector 1707E can be separated from the emitter chamber 1709A by a same distance as distance 1719. Detector chamber 1705E and/or detector 1707E can be separated from the emitter chamber 1709B by a same distance as distance 1719.

Detector chamber 1705C and/or detector 1707C can be separated from the emitter chamber 1709A by a distance 1721. Distance 1721 may extend from a geometric center of the emitter chamber 1709A to a geometric center of the detector chamber 1705C and/or to a geometric center of detector 1707C. Distance 1721 may be between about 6.5 mm and about 9.5 mm, between about 6.7 mm and about 9.3 mm, between about 6.9 mm and about 9.1 mm, between about 7.1 mm and about 8.9 mm, between about 7.3 mm and about 8.7 mm, between about 7.5 mm and about 8.5 mm, between about 7.7 mm and about 8.3 mm, between about 7.9 mm and about 8.2 mm, or any value or range between any of these values or ranges or any value or range bounded by any combination of these values, although values or ranges outside these values or ranges can be used in some cases. In some implementations, distance 1721 may be about 8.15 mm. Detector chamber 1705D and/or detector 1707D can be separated from the emitter chamber 1709A by a same distance as distance 1721. Detector chamber 1705A and/or detector 1707A can be separated from the emitter chamber 1709B by a same distance as distance 1721. Detector chamber 1705F and/or detector 1707F can be separated from the emitter chamber 1709B by a same distance as distance 1721.

Distance 1721 may be greater than distance 1719. Distance 1719 may be greater than distance 1717. Distance 1721 may be greater than distance 1717. Each of distances 1717, 1719, and 1721 may be different.

As shown and/or described, detector chamber 1705A and/or detector 1707A may be separated from emitter chamber 1709A by a different distance than emitter chamber 1709B. This may be the case for detector chambers 1705C, 1705D, and/or 1705F and/or for detectors 1707C, 1707D, and/or 1707F. Accordingly, optical radiation emanating from emitter chamber 1709A may travel a different (e.g., shorter) distance to arrive at detector chamber 1705A and/or detector 1707A than optical radiation emanating from emitter chamber 1709B. The depth to which optical radiation penetrates tissue may correspond to the distance it travels. For example, optical radiation travelling a longer distance may penetrate deeper into the tissue of a user than optical radiation travelling a shorter distance. In some implementations, depth of penetration may correspond linearly to distance travelled. In some implementations, depth of penetration may be twice a distance travelled. For example, light travelling 5 mm may penetrate tissue to a depth of 10 mm. The detectors can be modulated based on distance traveled by various optical radiation wavelengths. For example, detector 1707A can ignore optical radiation of a certain wavelength received from emitter chamber 1709A and can respond to optical radiation of that same wavelength when emitted from emitter chamber 1709B. For example, detector 1707A can respond to optical radiation having a green color (e.g., about 520 nm) when originating from emitter chamber 1709A and can ignore optical radiation at that same wavelength when originating from emitter chamber 1709B at least because optical radiation having a green color may not need to penetrate as deep into the tissue to provide accurate information and/or penetrating too deep may reduce information and/or accuracy. As another example, detector 1707A can respond to optical radiation having a red color or infrared radiation (e.g., between about 600 nm and about 1000 nm) when originating from emitter chamber 1709B and can ignore optical radiation at that same wavelength when originating from emitter chamber 1709A at least because optical radiation having a red color or infrared radiation that penetrates deeper into tissue may provide more information or more accurate information than if it does not penetrate as deep.

FIG. 17B is another front view of the sensor assembly 1700. Various rings 1731, 1733, 1735, 1737, 1739, and 1741 are shown as superimposed on the sensor assembly 1700. The rings 1731, 1733, 1735, 1737, 1739, and 1741 may be annular having respective radii and may be concentric with one another. The rings 1731, 1733, 1735, 1737, 1739, and 1741 may share a common center which may also coincide with a center of the sensor assembly 1700 and/or a center of the frame 1703.

Ring 1731 may encompass emitter chambers 1709A, 1709B. Ring 1731 may be tangential with at least a portion of an edge of emitter chamber 1709A and portion of an edge of emitter chamber 1709B. Ring 1731 may have a diameter between about 5.5 mm and about 8.5 mm, between about 5.7 mm and about 8.3 mm, between about 5.9 mm and about 8.1 mm, between about 6.1 mm and about 7.9 mm, between about 6.3 mm and about 7.7 mm, between about 6.5 mm and about 7.5 mm, between about 6.7 mm and about 7.3 mm, between about 6.9 mm and about 7.1 mm, or any value or range between any of these values or ranges or any value or range bounded by any combination of these values, although values or ranges outside these values or ranges can be used in some cases. Ring 1731 can have a diameter of at least 5 mm, at least 6 mm, at least 6.5 mm, at least 7 mm, at least 7.5 mm, at least 8 mm, or at least 8.5 mm. In some implementations, ring 1731 may have a diameter of about 7 mm.

Ring 1731 can have a surface area of πr², which in some cases may be about π3.5² mm=39 mm². Emitter chamber 1709A and emitter chamber 1709B may each have an optical radiation emitting area between about 10 mm² and about 20 mm², between about 14 mm² and about 18 mm², between about 14.2 mm² and about 17.8 mm², between about 14.4 mm² and about 17.6 mm², between about 14.6 mm² and about 17.4 mm², between about 14.8 mm² and about 17.2 mm², between about 15.0 mm² and about 17.0 mm², between about 15.2 mm² and about 16.8 mm², between about 15.4 mm² and about 16.6 mm², between about 15.6 mm² and about 16.4 mm², between about 15.8 mm² and about 16.2 mm², between about 16.0 mm² and about 16.2 mm², or any value or range between any of these values or ranges or any value or range bounded by any combination of these values, although values or ranges outside these values or ranges can be used in some cases. In some implementations, emitter chamber 1709A and emitter chamber 1709B may each have an area of about 16.13 mm². As used herein, the optical radiation emitting areas of the emitter chambers may refer to a surface area of a curved surface (e.g., a lens) covering the emitter chambers, and/or may refer to a cross sectional area of the emitter chambers, and/or may refer to a surface area of a portion of a PCB enclosed by the emitter chambers.

Emitter chamber 1709A and emitter chamber 1709B may have a total combined optical radiation emitting area between about 20 mm² and about 45 mm², between about 24 mm² and about 40 mm², between about 26 mm² and about 38 mm², between about 28 mm² and about 36 mm², between about 30 mm² and about 34 mm², between about 31 mm² and about 33 mm², or any value or range between any of these values or ranges or any value or range bounded by any combination of these values, although values or ranges outside these values or ranges can be used in some cases. In some implementations, emitter chamber 1709A and emitter chamber 1709B may have a total area of less than about 40 mm². In some implementations, emitter chamber 1709A and emitter chamber 1709B may have a total area of about 32.26 mm². Emitter chamber 1709A may have a same area as emitter chamber 1709B. The total optical radiation emitting area of emitter chamber 1709A and emitter chamber 1709B may be between about 50% and about 100% of the area of ring 1731, between about 60% and about 95% of the area of ring 1731, between about 70% and about 90% of the area of ring 1731, between about 80% and about 85% of the area of ring 1731, or any value or range between any of these values or ranges or any value or range bounded by any combination of these values, although values or ranges outside these values or ranges can be used in some cases. In some implementations, the total area of emitter chamber 1709A and emitter chamber 1709B may be about 82.7% of the area of ring 1731.

Emitter chamber 1709A can be separated from emitter chamber 1709B by a distance 1723. For example, an edge of emitter chamber 1709A can be parallel to an edge of emitter chamber 1069B which edges may be separated by distance 1723. Distance 1723 can be between about 0.4 mm and about 2.2 mm, between about 0.6 mm and about 2.0 mm, between about 0.8 mm and about 1.8 mm, between about 1.0 mm and about 1.6 mm, between about 1.1 mm and about 1.4 mm, between about 1.1 mm and about 1.3 mm, or any value or range between any of these values or ranges or any value or range bounded by any combination of these values, although values or ranges outside these values or ranges can be used in some cases. In some implementations, distance 1723 may be about 1.21 mm.

Ring 1733 may be tangential with at least a portion of an edge of detector chambers 1705A-1705F, such as an inner edge. Ring 1733 may have a diameter between about 7.4 mm and about 12.4 mm, between about 7.6 mm and about 12.2 mm, between about 7.8 mm and about 12.0 mm, between about 8.0 mm and about 11.8 mm, between about 8.2 mm and about 11.6 mm, between about 8.4 mm and about 11.4 mm, between about 8.6 mm and about 11.2 mm, between about 8.8 mm and about 11.0 mm, between about 9.0 mm and about 10.8 mm, between about 9.2 mm and about 10.6 mm, between about 9.4 mm and about 10.4 mm, between about 9.6 mm and about 10.4 mm, between about 9.8 mm and about 10.3 mm, between about 10.0 mm and about 10.3 mm, between about 10.1 mm and about 10.2 mm, or any value or range between any of these values or ranges or any value or range bounded by any combination of these values, although values or ranges outside these values or ranges can be used in some cases. Ring 1733 can have a diameter of at least 8 mm, at least 9 mm, at least 10 mm, at least 10.5 mm, at least 11 mm, at least 11.5 mm, or at least 12 mm. In some implementations, ring 1733 may have a diameter of about 10.14 mm.

Ring 1735 may intersect detector chambers 1705A-1705F and/or detectors 1707A-1707F. In some implementations, ring 1735 may bisect a width of respective detector chamber 1705A-1705F. For example, a width of detector chambers 1705A-1705F extending between ring 1733 and ring 1737 may by bisected by ring 1735. Likewise, ring 1735 may bisect detectors 1707A-1707F. A geometric center of respective detector chambers 1705A-1705F and/or a geometric center of respective detectors 1707A-1707F may lie on ring 1735. Ring 1735 may have a diameter between about 10 mm and about 16 mm, between about 10 mm and about 15 mm, between about 10.5 mm and about 14.5 mm, between about 11 mm and about 14 mm, between about 11.5 mm and about 14.0 mm, between about 12.0 mm and about 14.0 mm, between about 12.5 mm and about 13.5 mm, or any value or range between any of these values or ranges or any value or range bounded by any combination of these values, although values or ranges outside these values or ranges can be used in some cases. Ring 1735 can have a diameter of at least 10 mm, at least 11 mm, at least 11.5 mm, at least 12 mm, at least 12.5 mm, at least 13 mm, at least 13.5 mm, at least 14 mm, at least 15 mm, at least 16, or at least 17 mm. In some implementations, ring 1735 may have a diameter of about 12.9 mm.

The size of ring 1735 (with detectors 1707A-1707F positioned thereon) may correspond to a distance that optical radiation travels from emitter chambers 1709A, 1709B to respective detectors. As discussed herein, depth that optical radiation penetrates into tissue may be proportional to the distance it travels. Moreover, more power may be required to drive an emitter as distance increases in order to generate a significant signal at a detector. For example, an emitter may need to emit optical radiation at greater intensity for the optical radiation to be detected at greater distances than at shorter distances. Accordingly, a larger ring 1735 may cause optical radiation to travel a greater distance from emitters to detectors resulting in deeper tissue penetration (which may lead to more accurate measurements) but requiring greater power consumption. And a smaller ring 1735 may cause optical radiation to travel a short distance from emitters to detectors resulting in shallower tissue penetration (which may lead to less accurate measurements) but requiring less power consumption. Accordingly, ring 1735 (with detectors positioned thereon) may be sized as large as reasonable to maximize tissue penetration and measurement accuracy while adhering to power consumption restraints. Accordingly, if power consumption were not a factor, it may be desirable for ring 1735 to be larger than shown and/or described in order to generate the best measurements. Moreover, if measurement accuracy were less of a concern, it may be desirable for ring 1735 to be smaller than shown and/or described in order to reduce power consumption.

Ring 1737 may be tangential with at least a portion of an edge of detector chambers 1705A-1705F, such as an outer edge. Ring 1737 may enclose detector chambers 1705A-1705F. Ring 1737 may have a diameter between about 12 mm and about 20 mm, between about 13 mm and about 19 mm, between about 14 mm and about 18 mm, between about 14 mm and about 17 mm, between about 14.2 mm and about 16.8 mm, between about 14.4 mm and about 16.6 mm, between about 14.6 mm and about 16.4 mm, between about 14.8 mm and about 16.2 mm, between about 15.0 mm and about 16.0 mm, between about 15.2 mm and about 15.8 mm, between about 15.4 mm and about 15.8 mm, between about 15.6 mm and about 15.8 mm, or any value or range between any of these values or ranges or any value or range bounded by any combination of these values, although values or ranges outside these values or ranges can be used in some cases. Ring 1737 can have a diameter of at least 13 mm, at least 14 mm, at least 15 mm, at least 16 mm, at least 17 mm, at least 18 mm, or at least 19 mm. In some implementations, ring 1737 may have a diameter of about 15.79 mm.

A distance between ring 1737 and ring 1733 may correspond to a width of detector chambers 1705A-1705F. The distance between ring 1737 and ring 1733 may be a difference between any of the example radii of ring 1737 and ring 1733 shown and/or described herein. For example, the distance between ring 1737 and ring 1733 may be between about 1.8 mm and about 3.8 mm, between about 2.0 mm and about 3.6 mm, between about 2.2 mm and about 3.4 mm, between about 2.4 mm and about 3.2 mm, between about 2.6 mm and about 3.0 mm, between about 2.7 mm and about 2.9 mm, or any value or range between any of these values or ranges or any value or range bounded by any combination of these values, although values or ranges outside these values or ranges can be used in some cases. In some implementations, the distance between ring 1737 and ring 1733 may be about 2.82 mm.

Ring 1739 may be tangential with at least a portion of an edge of electrodes 1701A, 1701B, such as an inner edge. Ring 1739 may have a diameter between about 14 mm and about 23 mm, between about 15 mm and about 22 mm, between about 16 mm and about 21 mm, between about 17 mm and about 20 mm, between about 18 mm and about 19 mm, or any value or range between any of these values or ranges or any value or range bounded by any combination of these values, although values or ranges outside these values or ranges can be used in some cases. In some implementations, ring 1739 may have a diameter of about 18.5 mm.

Ring 1741 may be tangential with at least a portion of an edge of electrodes 1701A, 1701B, such as an outer edge. Ring 1741 may enclose electrodes 1701A, 1701B. Ring 1741 may have a diameter between about 20 mm and about 28 mm, between about 21 mm and about 27 mm, between about 22 mm and about 26 mm, between about 23 mm and about 25 mm, or any value or range between any of these values or ranges or any value or range bounded by any combination of these values, although values or ranges outside these values or ranges can be used in some cases. In some implementations, ring 1741 may have a diameter of about 24.7 mm.

A distance between ring 1741 and ring 1739 may correspond to a width of electrodes 1701A, 1701B. The distance between ring 1741 and ring 1739 may be a difference between any of the example radii of ring 1741 and ring 1739 shown and/or described herein. For example, the distance between ring 1741 and ring 1739 may be between about 1 mm and about 5 mm, between about 2 mm and about 4 mm, between about 2.2 mm and about 3.8 mm, between about 2.4 mm and about 3.6 mm, between about 2.6 mm and about 3.4 mm, between about 2.8 mm and about 3.3 mm, between about 3.0 mm and about 3.2 mm, or any value or range between any of these values or ranges or any value or range bounded by any combination of these values, although values or ranges outside these values or ranges can be used in some cases. In some implementations, the distance between ring 1737 and ring 1733 may be about 3.13 mm.

Perimeter 1743 may bound a portion of the sensor assembly 1700 that is configured to contact tissue of a user when worn by the user. The portion of the sensor assembly 1700 that is bounded by perimeter 1743 may be curved, for example convex or non-planar. Perimeter 1743 may circular. Perimeter 1743 may have a diameter between about 20 mm and about 40 mm, between about 22 mm and about 38 mm, between about 24 mm and about 36 mm, between about 26 mm and about 34 mm, between about 28 mm and about 32 mm, between about 28 mm and about 30 mm, between about 25 mm and about 35 mm, or any value or range between any of these values or ranges or any value or range bounded by any combination of these values, although values or ranges outside these values or ranges can be used in some cases. In some implementations, perimeter 1743 may have a diameter of about 29.15 mm. Perimeter 1743 can define a surface area of πr², which in some cases may be about π14.57² mm=667 mm².

The surface area of the portion of the sensor assembly 1700 configured to contact the tissue of a wearer (e.g., a surface of the sensor assembly 1700 bounded by perimeter 1743) may be between about 600 mm² and about 800 mm², between about 625 mm² and about 775 mm², between about 625 mm² and about 750 mm², between about 650 mm² and about 725 mm², between about 650 mm² and about 700 mm², between about 660 mm² and about 680 mm², between about 660 mm² and about 670 mm², between about 600 mm² and about 700 mm², or any value or range between any of these values or ranges or any value or range bounded by any combination of these values, although values or ranges outside these values or ranges can be used in some cases.

Any of detector chambers 1705A-1705F may have an optical radiation detecting area between about 5 mm² and about 50 mm², between about 7 mm² and about 40 mm², between about 9 mm² and about 30 mm², between about 10 mm² and about 25 mm², between about 12 mm² and about 20 mm², between about 13 mm² and about 19 mm², between about 14 mm² and about 18 mm², between about 15 mm² and about 17 mm², between about 15 mm² and about 16 mm², between about 15.25 mm² and about 15.75 mm², or any value or range between any of these values or ranges or any value or range bounded by any combination of these values, although values or ranges outside these values or ranges can be used in some cases. In some implementations, any of detector chambers 1705A-1705F may have an area of about 15.49 mm². One or more of the detector chambers 1705A-1705F may have a same area as each other. As used herein, the optical radiation detecting areas of the detector chambers may refer to a surface area of a curved surface (e.g., a lens) covering the detector chambers, and/or may refer to a cross sectional area of the detector chambers, and/or may refer to a surface area of a portion of a PCB enclosed by the detector chambers.

Detector chambers 1705A-1705F may have a total combined optical radiation detecting area between about 50 mm² and about 145 mm², between about 55 mm² and about 130 mm², between about 60 mm² and about 120 mm², between about 70 mm² and about 110 mm², between about 75 mm² and about 105 mm², between about 80 mm² and about 100 mm², between about 85 mm² and about 95 mm², or any value or range between any of these values or ranges or any value or range bounded by any combination of these values, although values or ranges outside these values or ranges can be used in some cases. In some implementations, detector chambers 1705A-1705F may have a total area of about 92.9 mm².

Any of the detector chambers 1705A-1705F may have an optical radiation detecting area that is less than (or in some cases, greater than) an optical radiation emitting area of any of the emitter chambers 1709A-1709B. Any of the detector chambers 1705A-1705F may have an optical radiation detecting area that is between about 75% and about 125%, between about 80% and about 115%, between about 85% and about 105%, between about 90% and about 100%, or between about 95% and about 100% of the optical radiation emitting area of any of the emitter chambers 1709A-1709B, or any value or range between any of these values or ranges or any value or range bounded by any combination of these values, although values or ranges outside these values or ranges can be used in some cases. In some implementations, any of the detector chambers 1705A-1705F may have an optical radiation detecting area that is about 96% of the optical radiation emitting area of any of the emitter chambers 1709A-1709B.

Any of the emitter chambers 1709A-1709B may have an optical radiation emitting area that is between about 10% and about 25%, between about 12% and about 23%, between about 14% and about 21%, between about 15% and about 20%, between about 16% and about 19%, between about 16% and about 18%, of the total optical radiation detecting area of the detector chambers 1705A-1705F, or any value or range between any of these values or ranges or any value or range bounded by any combination of these values, although values or ranges outside these values or ranges can be used in some cases. In some implementations, any of the emitter chambers 1709A-1709B may have an optical radiation emitting area that is about 17.4% of the total optical radiation detecting area of the detector chambers 1705A-1705F.

Detector chambers 1705A-1705F may have a total combined optical radiation detecting area between about 8% and about 20%, between about 9% and about 19%, between about 10% and about 18%, between about 11% and about 17%, between about 12% and about 16%, or between about 13% and about 15% of a surface area of the sensor assembly 1700 bounded by perimeter 1743, or any value or range between any of these values or ranges or any value or range bounded by any combination of these values, although values or ranges outside these values or ranges can be used in some cases. In some implementations, detector chambers 1705A-1705F may have a total combined optical radiation detecting area of about 13.9% of a surface area of the sensor assembly 1700 bounded by perimeter 1743.

Emitter chambers 1709A-1709B may have a total combined optical radiation emitting area between about 3% and about 7%, between about 3.2% and about 6.8%, between about 3.4% and about 6.6%, between about 3.6% and about 6.4%, between about 3.8% and about 6.2%, between about 4.0% and about 5.0%, or between about 4.5% and about 5.0% of a surface area of the sensor assembly 1700 bounded by perimeter 1743, or any value or range between any of these values or ranges or any value or range bounded by any combination of these values, although values or ranges outside these values or ranges can be used in some cases. In some implementations, emitter chambers 1709A-1709B may have a total combined optical radiation emitting area of about 4.8% of a surface area of the sensor assembly 1700 bounded by perimeter 1743.

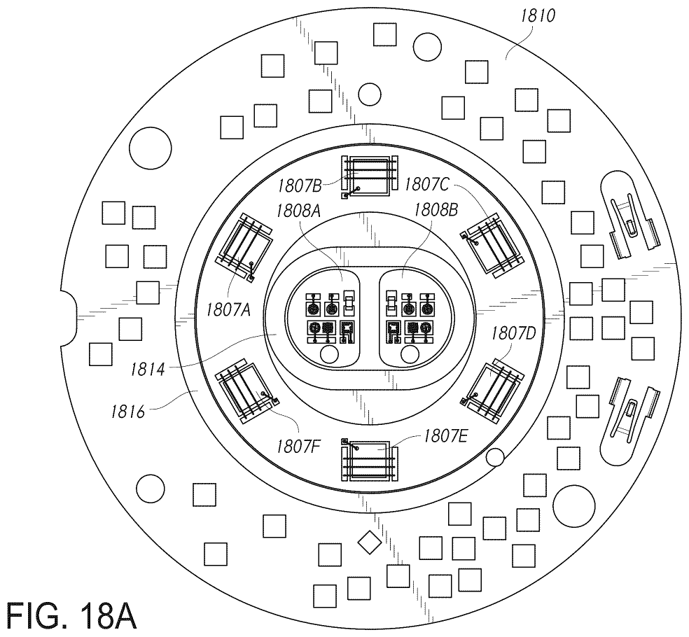

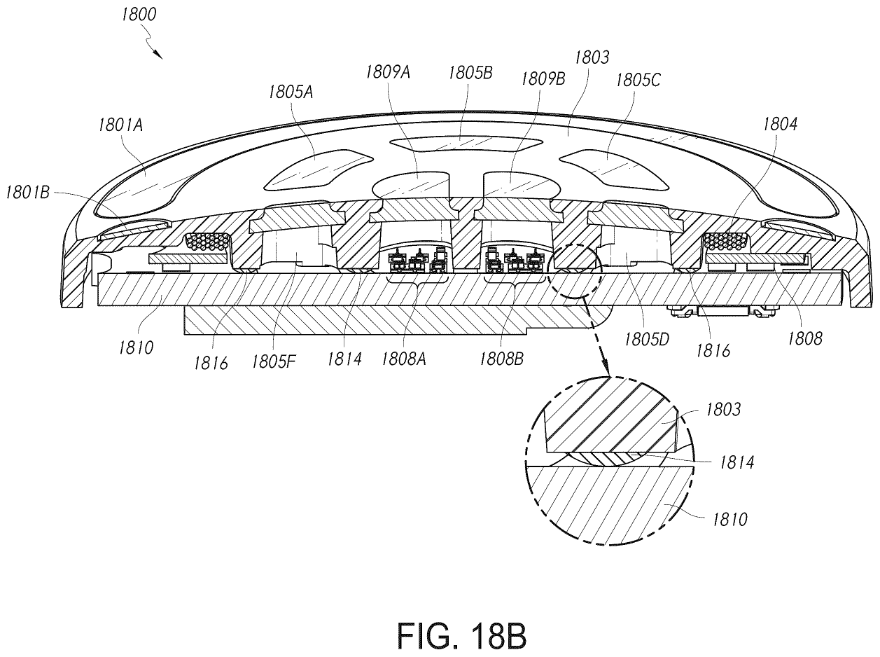

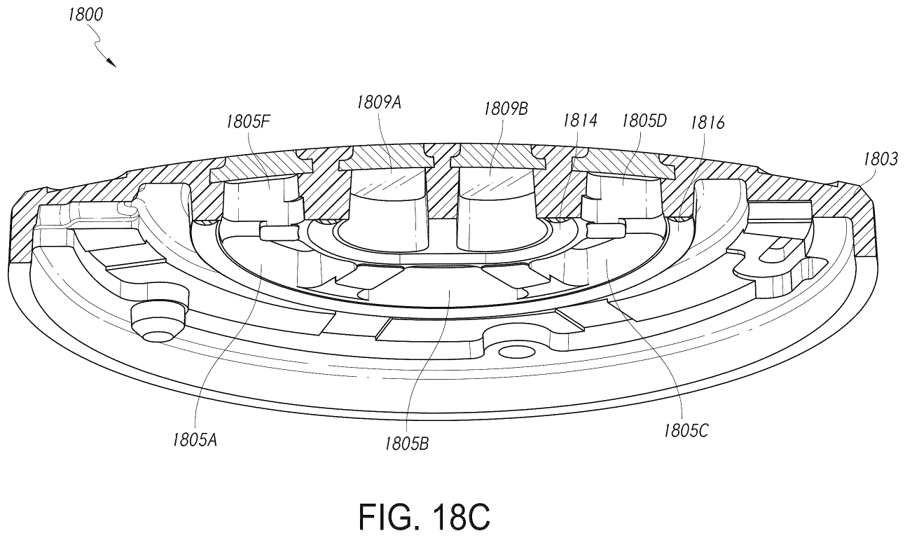

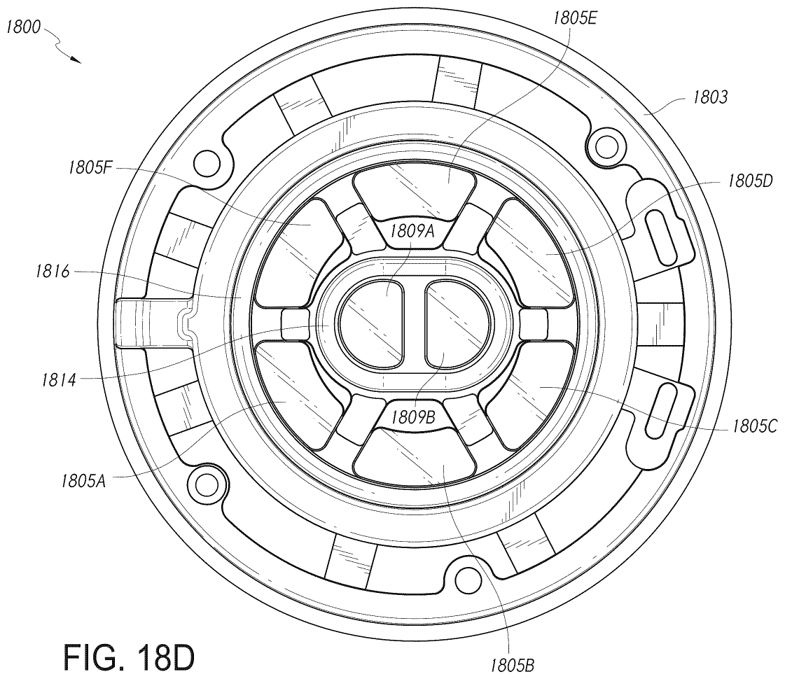

FIG. 18A is a front view of an example substrate 1810 which may be implemented in any of the example sensor assemblies shown and/or described herein. The substrate 1810 may be a printed circuit board (PCB). One or more detectors 1807A-1807F may be positioned on the substrate 1810. A first group of emitters 1808A may be positioned on the substrate 1810. A second group of emitters 1808B may be positioned on the substrate 1810. Each of the first and second groups of emitters 1808A, 1808B may comprise one or more emitters, such as one, two, three, four, or five emitters. A temperature sensor, such as a thermistor, may be positioned on the substrate 1810, such as adjacent to the first group of emitters 1808A. Another temperature sensors may be positioned on the substrate 1810, such as adjacent to the second group of emitters 1808B.

A substance 1814 may be positioned on the substrate 1810. The substance 1814 can surround the first group of emitters 1808A and/or the second group of emitters 1808B. The substance 1814 can entirely enclose the first group of emitters 1808A and/or the second group of emitters 1808B. The substance 1814 may form a closed loop on the substrate 1810 such as around the emitters 1808A, 1808B. The substance 1814 may be opaque such that the substance 1814 prevents, inhibits, and/or reduces an amount of optical radiation transmitted through the substance 1814. For example, the substance 1814 may absorb all, substantially all, and/or a majority of optical radiation incident on the substance 1814. The substance 1814 may be black. The substance 1814 may inhibit optical radiation, such as light, from passing from the first group of emitters 1808A and/or the second group of emitters 1808B along the substrate 1810 to the detectors 1807A-1807F. In some implementations, a substance, such as a portion of substance 1814, may be positioned on the substrate 1810 between the first and second groups of emitters 1808A-1808B.

The substance 1814 may be adhesive and may adhere to the substrate 1810 and/or may adhere other materials to the substrate 1810. The substance 1814 may be deformable. For example, the substance 1814 may deform or change shape when the substance 1814 is applied to the substrate 1810 and/or when other materials (e.g., a frame) contact the substrate 1814. The substance 1814 may change viscosity, rigidity and/or deformability with time. For example, the substance 1814 may become more rigid or less deformable as the substance 1814 dries after the substance is initially applied to the substrate 1810. The substance 1814 may be a liquid (with a higher viscosity than water) when initially applied to the substrate 1810 which can solidify with time after applied to the substrate 1810. The substance 1814 may be a solid such as a foam which can be compressed. The substance 1814 may be a glue and can have one or more pigments or dyes (e.g., black) distributed therein to provide opacity to the substance 1814.

A substance 1816 may be positioned on the substrate 1810. The substance 1816 may comprise one or more of the characteristics of substance 1814. The substance 1816 may be a same material as substance 1814. The substance 1816 may be opaque and may inhibit light from passing along the substrate 1810 to the detectors 1807A-1807F. The substance 1816 may surround the detectors 1807A-1807F. The substance 1816 may entirely enclose the detectors 1807A-1807F. The substance 1816 may form a closed loop on the substrate 1810 such as around the detectors 1807A-1807F. The detectors 1807A-1807F may be positioned on the substrate 1810 between substance 1814 and substance 1816. The substance 1816 may enclose the substance 1814. In some implementations, a substance, such as a portion of substance 1816 and/or 1814, may be positioned on the substrate 1810 between one or more of the detector chambers 1807A-1807F.

In some implementations, during manufacturing the substance 1814 and/or substance 1816 may be applied to the substrate 1810 and then a frame may be positioned on the substrate 1810 and adhered to the substance 1814 and substance 1816.

FIG. 18B is a side cutaway view of an example sensor assembly 1800 which can include a frame 1803 and a substrate 1810. Electrodes 1801A-1801B can be positioned on the frame 1803. The frame 1803 can house an induction coil 1804 and an induction plate 1808 positioned adjacent to the induction coil 1804. The induction plate 1808 may be made of a ferrite material and may enhance inductive charging by increasing the strength of magnetic fields.

The frame 1803 can include detector chambers 1805A-1805F and emitter chambers 1809A-1809B. Emitter chamber 1809A can house a first group of emitters 1808A positioned on the substrate 1810. Emitter chamber 1809B can house a second group of emitters 1808B positioned on the substrate 1810. Detector chambers 1805A-1805F can house detectors. The frame 1803 can inhibit optical radiation from passing between chambers such as from emitters chambers 1809A-1809B to detector chambers 1805A-1805F without passing through tissue of a wearer. The frame 1803 may induce optical radiation emanating from emitter chambers 1809A-1809F to penetrate tissue of wearer before entering detector chambers 1805A-1805F. The frame 1803 may inhibit or reduce an amount optical radiation that travels between any of the detector chambers 1805A-1805F.

The frame 1803 can be positioned on the substrate 1810. The frame 1803 may be coupled to a substantially planar surface of the substrate 1810, as shown. However, due to variabilities in materials and/or manufacturing processes, the surfaces of the frame 1803 and the substrate 1810 that contact each other may not be perfectly planar or flat. Accordingly, gaps may exist at or around the portions of the frame 1803 and the substrate 1810 that contact each other. Such gaps may allow optical radiation to pass between the frame 1803 and the substrate 1810 which may undesirably affect physiological measurements such as by allowing optical radiation to pass from emitter chambers 1809A-1809B to detector chambers 1805A-1805F without travelling through tissue of the wearer.

The frame 1803 and substrate 1810 may be secured together at least by an adhesion force from substance 1814 and/or substance 1816 which may be an opaque adhesive. Substance 1814 and/or substance 1816 can fill some or all gaps between frame 1803 and substrate 1810. Substance 1814 can inhibit optical radiation from passing from emitter chambers 1809A-1809B to detector chambers 1805A-1805F that would otherwise be able to pass through any gaps between frame 1803 and substrate 1810. Substance 1816 can inhibit optical radiation from entering detector chambers 1805A-1805F that would otherwise be able to pass through any gaps between frame 1803 and substrate 1810.

Substance 1814 and substance 1816 are illustrated as shown in FIG. 18B for clarity and ease of understanding. For example, in some implementations, frame 1803 and substrate 1810 may be positioned much closer to each other than shown in FIG. 18B such that substance 1814 and substance 1816 may be deformed into a much flatter configuration that might be difficult to view. FIG. 18B may illustrate the sensor assembly 1800 in a state during a manufacturing process such as when the frame 1803 and substrate 1810 are being joined together. For example, applying a downward force to the frame 1803 and/or an upward force to the substrate 1810 may cause the frame 1803 and substrate 1810 to move closer together and deform the substance 1814 and substance 1816 to occupy gaps between frame 1803 and substrate 1810 and to be flatter than is currently shown in FIG. 18B. Substance 1814 and/or 1816 can adhere to substrate 1810 and/or frame 1803 and can thereby cause substrate 1810 and frame 1803 to be coupled to each other.

FIG. 18C illustrates a cutaway perspective view of an interior region of a frame 1803 of a sensor assembly 1800. Substance 1814 may contact the frame 1803 and may surround emitter chambers 1809A-1809B in a closed loop. Substance 1814 may be positioned on the frame 1803 between the emitter chambers 1809A-1809B and the detector chambers 1805A-1805F. Substance 1816 may contact the frame 1803 and may surround the detector chambers 1805A-1805F in a closed loop. In some implementations, during manufacturing the substance 1814 and/or substance 1816 may be applied to the frame 1803 and a substrate may then be positioned on the frame 1803 and adhered to the substance 1814 and substance 1816.

FIG. 18D is a front view of an interior region of a frame 1803 of a sensor assembly 1800. Substance 1814 may contact the frame 1803 and may surround emitter chambers 1809A-1809B in a closed loop. Substance 1814 may be positioned on the frame 1803 between the emitter chambers 1809A-1809B and the detector chambers 1805A-1805F. Substance 1816 may contact the frame 1803 and may surround the detector chambers 1805A-1805F in a closed loop.

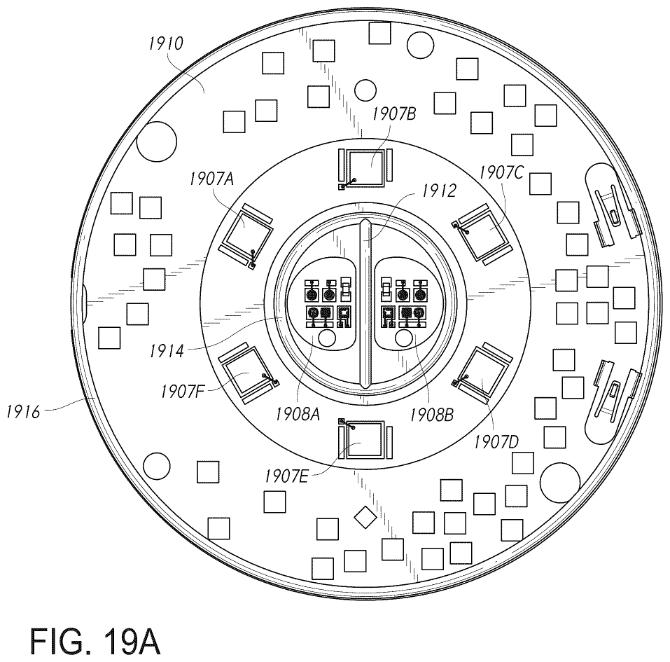

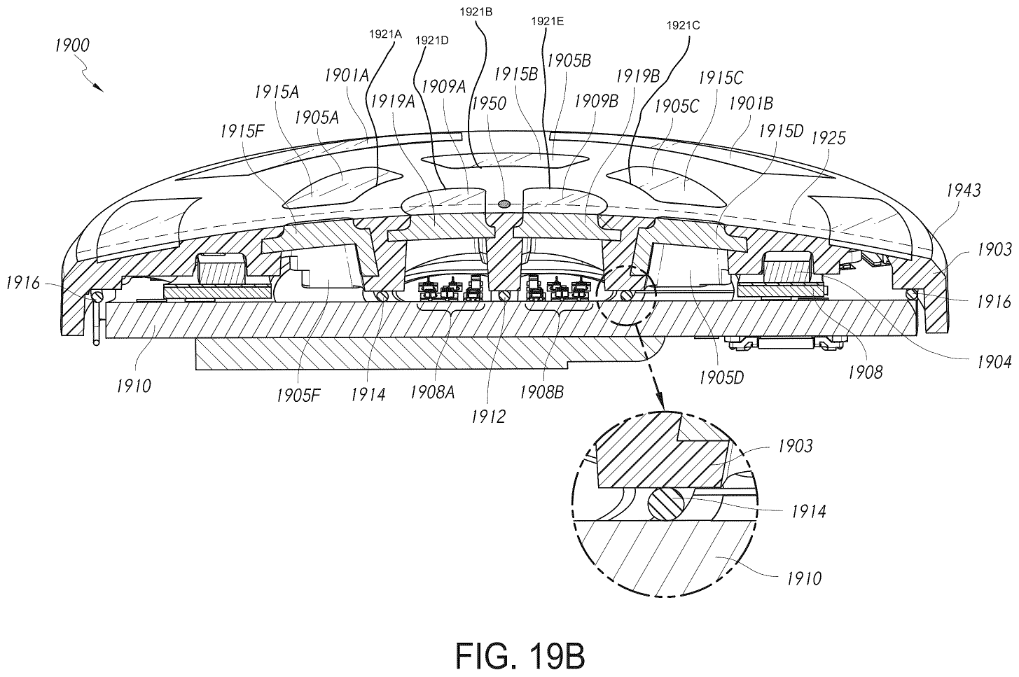

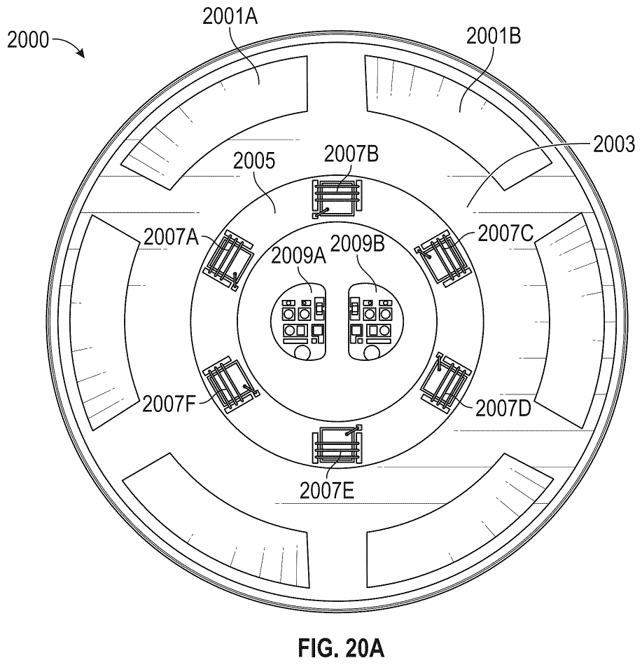

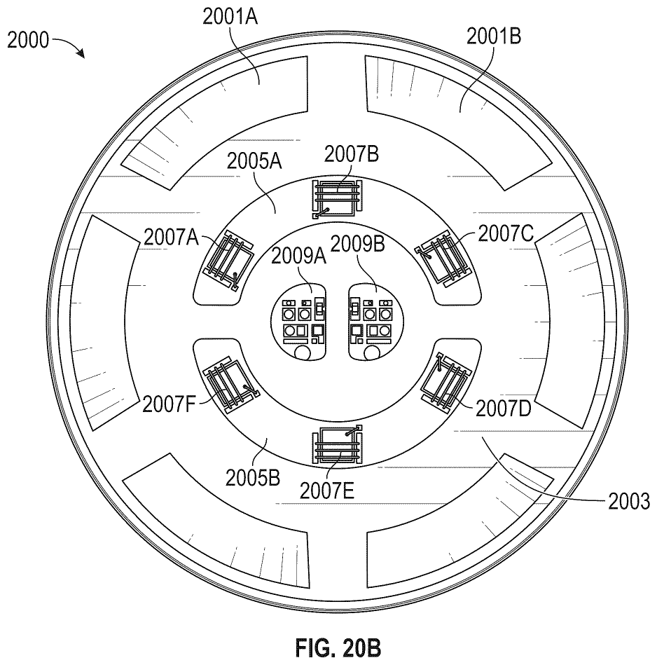

FIG. 19A is a front view of an example substrate 1910 which may be implemented in any of the example sensor assemblies shown and/or described herein. The substrate 1910 may be a printed circuit board (PCB). One or more detectors 1907A-1907F may be positioned on the substrate 1910. A first group of emitters 1908A may be positioned on the substrate 1910. A second group of emitters 1908B may be positioned on the substrate 1910. Each of the first and second groups of emitters 1908A, 1908B may comprise one or more emitters, such as one, two, three, four, or five emitters. A temperature sensor, such as a thermistor, may be positioned on the substrate 1910, such as adjacent to the first group of emitters 1908A. Another temperature sensors may be positioned on the substrate 1910, such as adjacent to the second group of emitters 1908B.

A substance 1914 may be positioned on the substrate 1910. The substance 1914 can surround the first group of emitters 1908A and/or the second group of emitters 1908B. The substance 1914 can entirely enclose the first group of emitters 1908A and/or the second group of emitters 1908B. The substance 1914 may form a closed loop on the substrate 1910 such as around the first group of emitters 1908A and/or the second group of emitters 1908B. The substance 1914 may be opaque such that the substance 1914 prevents, inhibits, and/or reduces an amount of optical radiation transmitted through the substance 1914. For example, the substance 1914 may absorb all, substantially all, and/or a majority of optical radiation incident on the substance 1914. The substance 1914 may be black. The substance 1914 may inhibit optical radiation, such as light, from passing from the first group of emitters 1908A and/or the second group of emitters 1908B along the surface of the substrate 1910 to the detectors 1907A-1907F.

The substance 1914 may be adhesive and may adhere to the substrate 1910 and/or may adhere other materials to the substrate 1910. The substance 1914 may be deformable. For example, the substance 1914 may deform or change shape when the substance 1914 is applied to the substrate 1910 and/or when other materials (e.g., a frame) contact the substrate 1914. The substance 1914 may change rigidity and/or deformability with time. For example, the substance 1914 may become more rigid or less deformable as the substance 1914 dries after the substance is initially applied to the substrate 1910. The substance 1914 may be a liquid which can solidify with time. The substance 1914 may be a solid such as a foam which can be compressed.

A substance 1912 may be positioned on the substrate 1910. The substance 1912 may comprise one or more of the characteristics of substance 1914. The substance 1912 may be a same material as substance 1914. The substance 1912 may be opaque and may inhibit light from passing between emitter chambers 1809A-1809B along substrate 1910. The substance 1912 and substance 1914 may contact each other and may be from a single continuous substance. In this example, substance 1914 and substance 1912 form three closed loops, one loop around the first group of emitters 1908A, a second loop around the second group of emitters 1908B, and a third loop around both the first and second groups of emitters 1908A, 1908B. In this example, the closed loop around the first group of emitters 1908A touches the closed loop around the second group of emitters 1908B by virtue of substance 1912 forming a portion of each of those two closed loops. In some implementations, substance may be arranged on the substrate 1910 that forms a closed loop that surrounds the first group of emitters 1908A and that also forms a second closed loop that surrounds the second group of emitters 1908B without forming a third closed loop around both first and second groups of emitter 1908A, 1908B and/or without the two closed loops touching each other.

A substance 1916 may be positioned on the substrate 1910. The substance 1916 may comprise one or more of the characteristics of substance 1914. The substance 1916 may be a same material as substance 1914. The substance 1916 may be opaque and may inhibit light from passing to the detectors 1907A-1907F. The substance 1916 may be adhesive. The substance 1916 may surround the detectors 1907A-1907F. The substance 1916 may entirely enclose the detectors 1907A-1907F. The substance 1916 may form a closed loop on the substrate 1910. The detectors 1907A-1907F may be positioned on the substrate 1910 between the substance 1914 and the substance 1916. The substance 1916 may enclose the substance 1914. In some implementations, a substance, such as a portion of substance 1916 and/or 1914, may be positioned on the substrate 1810 between one or more of the detector chambers 1907A-1907F. Any of the substances shown and/or described herein, such as substances 1814, 1816, 1912, 1914, and/or 1916 can have any of the properties, characteristics, structural features, and/or operational features, and may even be the same material as any of the other substances shown and/or described herein.

FIG. 19B is a side cutaway view of an example sensor assembly 1900 which can include a frame 1903 and a substrate 1910. Electrodes 1901A-1901B can be positioned on the frame 1903. The frame 1903 can house an induction coil 1904 and an induction plate 1908 positioned adjacent to the induction coil 1904. The induction plate 1908 may be made of a ferrite material and may enhance inductive charging by increasing the strength of magnetic fields.

The frame 1903 can include detector chambers 1905A-1905F and emitter chambers 1909A-1909B. Emitter chamber 1909A can house a first group of emitters 1908A positioned on the substrate 1910. Emitter chamber 1909B can house a second group of emitters 1908B positioned on the substrate 1910. Detector chambers 1905A-1905F can house detectors. The frame 1903 can inhibit optical radiation from passing between chambers such as from emitters chambers 1909A-1909B to detector chambers 1905A-1905F without passing through tissue of a wearer. The frame 1903 may induce optical radiation emanating from emitter chambers 1909A-1909F to penetrate tissue of wearer before entering detector chambers 1905A-1905F. The frame 1903 may inhibit or reduce an amount optical radiation that travels between any of the detector chambers 1905A-1905F.

The frame 1903 can be positioned on the substrate 1910. The frame 1903 may be coupled to a substantially planar surface of the substrate 1910, as shown. However, due to variabilities in materials and/or manufacturing processes, the surfaces of the frame 1903 and the substrate 1910 that contact each other may not be perfectly planar or flat. Accordingly, gaps may exist at or around the portions of the frame 1903 and the substrate 1910 that contact each other. Such gaps may allow optical radiation to pass between the frame 1903 and the substrate 1910 which may undesirably affect physiological measurements such as be allowing optical radiation to pass from emitter chambers 1909A-1909B to detector chambers 1905A-1905F without travelling through tissue of the wearer.

The frame 1903 and substrate may be secured together by substance 1912, substance 1914, and/or substance 1916 which may be adhesive. Substance 1912, substance 1914 and/or substance 1916 can fill any gaps between frame 1903 and substrate 1910. Substance 1912 can inhibit optical radiation from passing between emitter chambers 1909A-1909B that would otherwise be able to pass through any gaps between frame 1903 and substrate 1910. Substance 1914 can inhibit optical radiation from passing from emitter chambers 1909A-1909B to detector chambers 1905A-1905F that would otherwise be able to pass through any gaps between frame 1903 and substrate 1910. Substance 1916 can inhibit optical radiation from entering detector chambers 1905A-1905F that would otherwise be able to pass through any gaps between frame 1903 and substrate 1910.

Substance 1912, substance 1914, and substance 1916 are illustrated as shown in FIG. 19B for clarity and ease of understanding. For example, in some implementations, frame 1903 and substrate 1910 may be positioned much closer to each other than shown in FIG. 19B such that substance 1912, substance 1914, and substance 1916 may be deformed into a much flatter configuration that might be difficult to view. FIG. 19B may illustrate the sensor assembly 1900 in a state during a manufacturing process such as when the frame 1903 and substrate 1910 are being joined together. For example, applying a downward force to the frame 1903 and/or an upward force to the substrate 1910 may cause the frame 1903 and substrate 1910 to move closer together and deform the substance 1912, substance 1914, and substance 1916 to occupy gaps between frame 1903 and substrate 1910 and to be flatter than is currently shown in FIG. 19B.

In some implementations, during manufacturing the substance 1914, substance 1912, and/or substance 1916 may be applied to the substrate 1910 and the frame 1903 may then be positioned on the substrate 1910 and adhered to the substance 1914, substance 1912, and substance 1916. In some implementations, during manufacturing the substance 1914, substance 1912, and/or substance 1916 may be applied to the frame 1903 and the substrate 1910 may then be positioned on the frame 1903 and adhered to the substance 1914, substance 1912, and substance 1916.

Emitter lenses 1919A,1919B can be incorporated into frame 1903. Emitter lenses 1919A,1919B may be separate components from frame 1903. Frame 1903 can be molded around emitter lenses 1919A, 1919B. Frame 1903 and emitter lenses 1919A, 1919B may be made of different materials. Frame 1903 and emitter lenses 1919A,1919B may be made of plastic or in some cases may be formed of and/or comprise the same material (e.g., formed at different stages of manufacturing). Emitter lenses 1919A, 1919B may be made of polycarbonate. Emitter lenses 1919A,1919B can be transparent or translucent and can allow optical radiation to pass therethrough.

Detector lenses 1915A-1915F can be incorporated into frame 1903. Detector lenses 1915A-1915F may be separate components from frame 1903. Frame 1903 can be molded around detector lenses 1915A-1915F. Frame 1903 and detector lenses 1915A-1915F may be made of different materials or in some cases may be formed of and/or comprise the same material (e.g., formed at different stages of manufacturing). Frame 1903 and/or detector lenses 1915A-1915F may be made of plastic or glass. Detector lenses 1915A-1915F may be made of polycarbonate. Detector lenses 1915A-1915F can be transparent or translucent and can allow optical radiation to pass therethrough.

Emitter lenses 1919A,1919B may have a greater opacity than detector lenses 1915A-1915F which may allow the emitter lenses 1919A,1919B to diffuse light more than detector lenses 1915A-1915F. The detector lenses 1915A-1915F can have texture that diffuses optical radiation.

In this example, frame 1903 includes edges 1921 that form closed loops defining windows of the detector chambers and/or emitter chambers. For example, edge 1921A defines a window of detector chamber 1905A and edge 1921D defines a window of emitter chamber 1909A. The edges 1921 can directly contact the skin of the wearer when the sensor assembly 1900 is worn. This may allow the frame 1903 to directly contact the skin of the wearer at region between the detector lenses 1915, and/or emitter lenses 1919 which can inhibit optical radiation from traveling along the user's skin between the detector lenses 1915, and/or emitter lenses 1919. In this example, the sensor assembly does not include any other material overlaid on the frame 1903, detector lenses 1915, and/or emitter lenses 1919 which reduces light piping along the user's skin.

A surface of the frame 1903 may be flush with a surface of the emitter lenses 1919A-1919B and a surface of the detector lenses 1915A-1915F such that a surface of the sensor assembly 1900 configured to contact the skin of a wearer forms a smooth continuous surface. The surface of any of the emitter lenses 1919A-1919B, the detector lenses 1915A-1915F, and the frame 1903 may contact the skin of a wearer when the sensor assembly 1900 is worn by the wearer. For example, the sensor assembly 1900 may not comprise a layer of material (e.g., a light transmissive material) between the frame 1903 and the user's skin, between the emitter lenses 1919A-1919B and the user's skin, or between the detector lenses 1915A-1915F and the user's skin. Advantageously, allowing the frame 1903 to directly contact the user's skin (e.g., by omitting a light transmissive layer between the frame 1903 and the skin) may inhibit optical radiation from travelling from emitter chambers 1909A-1909B to the detector chambers 1905A-1905F without travelling through the user's tissue. For example, when the frame 1903 contacts the user's tissue the frame 1903 may block optical radiation until the frame 1903 ends where it contacts the user's tissue forcing optical radiation to travel through the tissue whereas adding another layer between the frame 1903 and the skin might allow the additional layer to conduct optical radiation through additional layer between the skin and the frame 1903 thus allowing the optical radiation to travel along the frame 1903 without penetrating the tissue. Thus the sensor assembly 1900 may not have a light transmissive material, such as plastic or glass, covering the frame 1903.

A surface of the emitter lens 1919A configured to contact the skin of a wearer when the sensor assembly 1900 is worn may be curved (e.g., non-planar) and may have an area of between about 8 mm² and about 11 mm², between about 8.2 mm² and about 10.8 mm², between about 8.4 mm² and about 10.6 mm², between about 8.6 mm² and about 10.4 mm², between about 8.8 mm² and about 10.2 mm², between about 9.0 mm² and about 10.0 mm², between about 9.2 mm² and about 9.8 mm², between about 9.4 mm² and about 9.7 mm², between about 9.55 mm² and about 9.75 mm², or any value or range between any of these values or ranges or any value or range bounded by any combination of these values, although values or ranges outside these values or ranges can be used in some cases. In some implementations, a surface of the emitter lens 1919A configured to contact the skin of the user may have an area of about 9.67 mm². A surface of the emitter lens 1919B configured to contact the skin of a wearer when the sensor assembly 1900 is worn may be curved (e.g., non-planar) and may have an area that similar or the same as the surface area of emitter lens 1919B described herein.

Each of the detector lenses 1915A-1915F may have a surface configured to contact the skin of a wearer when the sensor assembly 1900 is worn that may be curved (e.g., non-planar) and may have an area of between about 10 mm² and about 16 mm², between about 10 mm² and about 14 mm², between about 10.25 mm² and about 14 mm², between about 10.5 mm² and about 14 mm², between about 10.75 mm² and about 14 mm², between about 11 mm² and about 14 mm², between about 11.25 mm² and about 14 mm², between about 11.5 mm² and about 14 mm², between about 11.75 mm² and about 14 mm², between about 12 mm² and about 14 mm², between about 10 mm² and about 13.75 mm², between about 10 mm² and about 13.5 mm², between about 10 mm² and about 13.25 mm², between about 10 mm² and about 13 mm², between about 10 mm² and about 12.75 mm², between about 10 mm² and about 12.5 mm², between about 11 mm² and about 13 mm², between about 11.5 mm² and about 12.75 mm², between about 12 mm² and about 12.5 mm², or any value or range between any of these values or ranges or any value or range bounded by any combination of these values, although values or ranges outside these values or ranges can be used in some cases. In some implementations, any of detector lenses 1915A-1915F may have a surface configured to contact the skin of a user with an area of about 12.258 mm².

A surface of the sensor assembly 1900 configured to contact the skin of a wearer may be bounded by perimeter 1943. Perimeter 1943 may be circular. Perimeter 1943 may have a diameter between about 20 mm and about 40 mm, between about 22 mm and about 38 mm, between about 24 mm and about 36 mm, between about 26 mm and about 34 mm, between about 28 mm and about 32 mm, between about 28 mm and about 30 mm, between about 25 mm and about 35 mm, or any value or range between any of these values or ranges or any value or range bounded by any combination of these values, although values or ranges outside these values or ranges can be used in some cases. In some implementations, perimeter 1943 may have a diameter of about 29.3 mm.

Centerline 1925 is shown as superimposed on sensor assembly 1900. The centerline 1925 may bisect the sensor assembly 1900. Center point 1950 is shown as superimposed on sensor assembly 1900. The center point 1950 may be superimposed on a geometric center of the sensor assembly 1900 which may also coincide with a geometric center of the perimeter 1943. In other words, every point along perimeter 1943 may be the same distance from center point 1950. The center line 1925 intersects the center point 1950. The center line 1925 may define a portion of a circle or approximate circle. For example, the center line 1925 may extend along a circular path resulting from the curved surface of the sensor assembly 1900 with a center of the circle defined by the centerline 1925 being located downward from the sensor assembly 1900 with respect to the page.

A circle defined, at least partially, by the center line 1925 may have a radius of between about 1.0 inches and about 6.0 inches, between about 1.0 inches and about 4.0 inches, between about 1.2 inches and about 3.8 inches, between about 1.4 inches and about 3.6 inches, between about 1.6 inches and about 3.4 inches, between about 1.8 inches and about 3.2 inches, between about 2.0 inches and about 3.0 inches, between about 2.2 inches and about 2.8 inches, between about 2.4 inches and about 2.6 inches, or any value or range between any of these values or ranges or any value or range bounded by any combination of these values, although values or ranges outside these values or ranges can be used in some cases. In some implementations, a circle defined, at least partially, by the center line 1925 may have a radius of about 2.5 inches.

A circle defined, at least partially, by the center line 1925 may have a radius of between about 2.0 inches and about 6.0 inches, between about 2.2 inches and about 5.8 inches, between about 2.4 inches and about 5.6 inches, between about 2.6 inches and about 5.4 inches, between about 2.8 inches and about 5.2 inches, between about 3.0 inches and about 5.0 inches, between about 3.2 inches and about 4.8 inches, between about 3.4 inches and about 4.6 inches, between about 3.6 inches and about 4.4 inches, between about 3.8 inches and about 4.2 inches, between about 3.8 inches and about 4.0 inches, or any value or range between any of these values or ranges or any value or range bounded by any combination of these values, although values or ranges outside these values or ranges can be used in some cases. In some implementations, a circle defined, at least partially, by the center line 1925 may have a radius of about 3.82 inches.

In some implementations, a circle defined, at least partially, by the center line 1925 may have a radius that is greater than 4 inches, greater than 5 inches, greater than 6 inches, including up to an infinite radius meaning the surface of the sensor assembly 1800 is flat or planar.

The distance between the center point 1950 and the substrate 1910 may be the greatest distance between any point on the frame 1903 and the substrate 1910. In other words, the frame 1903 may have the greatest height at the center point 1950. A distance between center point 1950 and the substrate 1910 (e.g., a shortest distance), which may also correspond to a maximum height of the frame 1903, may be between about 2.0 mm and about 4.0 mm, between about 2.2 mm and about 3.8 mm, between about 2.4 mm and about 3.6 mm, between about 2.6 mm and about 3.4 mm, between about 2.8 mm and about 3.2 mm, between about 2.8 mm and about 3.0 mm, or any value or range between any of these values or ranges or any value or range bounded by any combination of these values, although values or ranges outside these values or ranges can be used in some cases. In some implementations, a distance between center point 1950 and the substrate 1910 (e.g., a shortest distance) may be about 2.87 mm.

A distance between center point 1950 and the substrate 1910 (e.g., a shortest distance), which may also correspond to a maximum height of the frame 1903, may be between about 1.5 mm and about 3.5 mm, between about 1.7 mm and about 3.3 mm, between about 1.9 mm and about 3.1 mm, between about 2.1 mm and about 2.9 mm, between about 2.3 mm and about 2.7 mm, between about 2.4 mm and about 2.7 mm, between about 2.5 mm and about 2.7 mm, or any value or range between any of these values or ranges or any value or range bounded by any combination of these values, although values or ranges outside these values or ranges can be used in some cases. In some implementations, a distance between center point 1950 and the substrate 1910 (e.g., a shortest distance) may be about 2.59 mm.

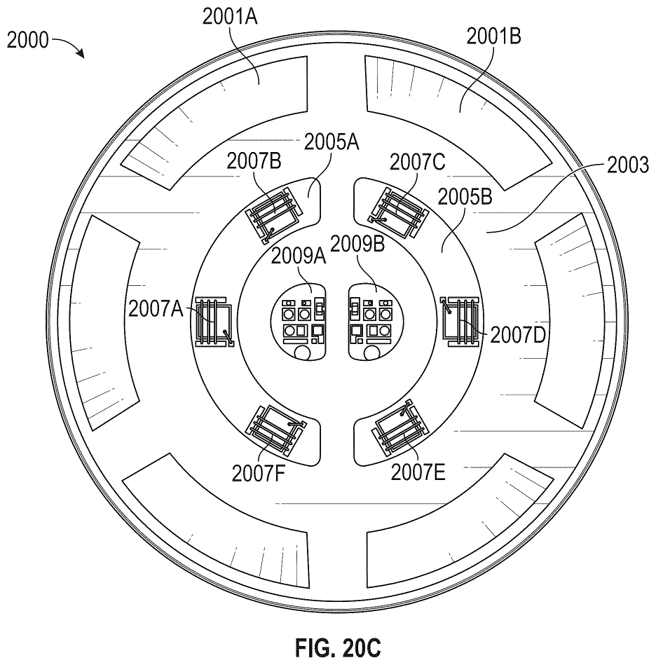

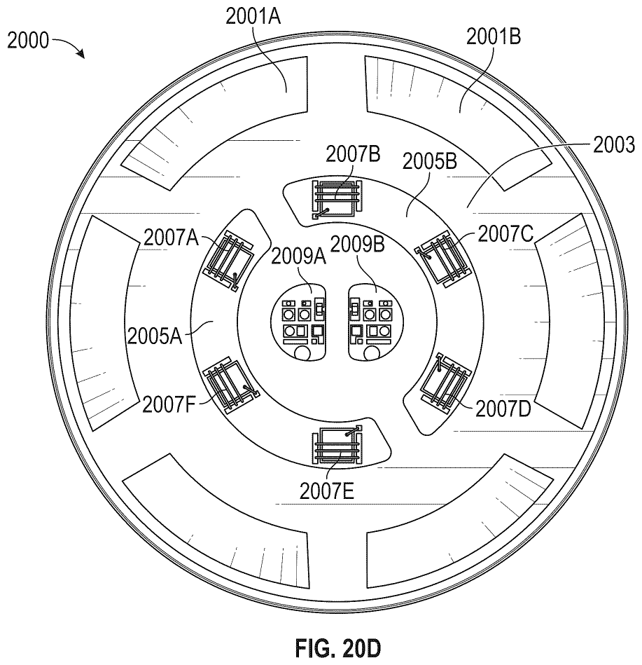

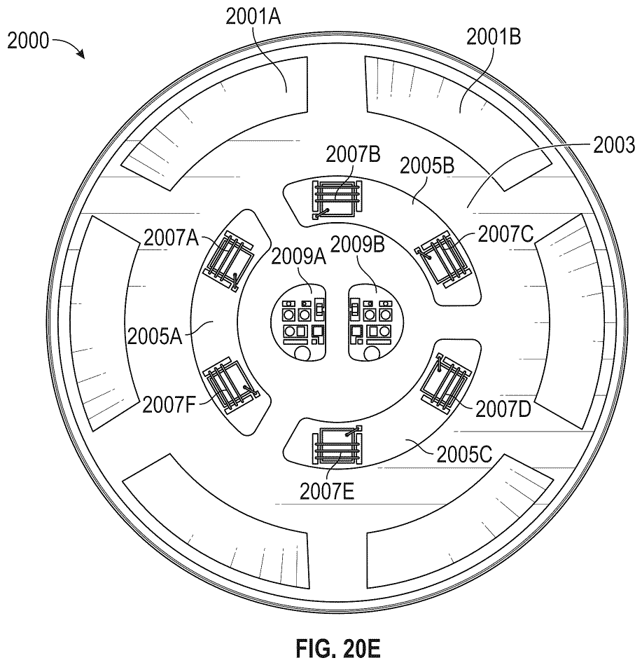

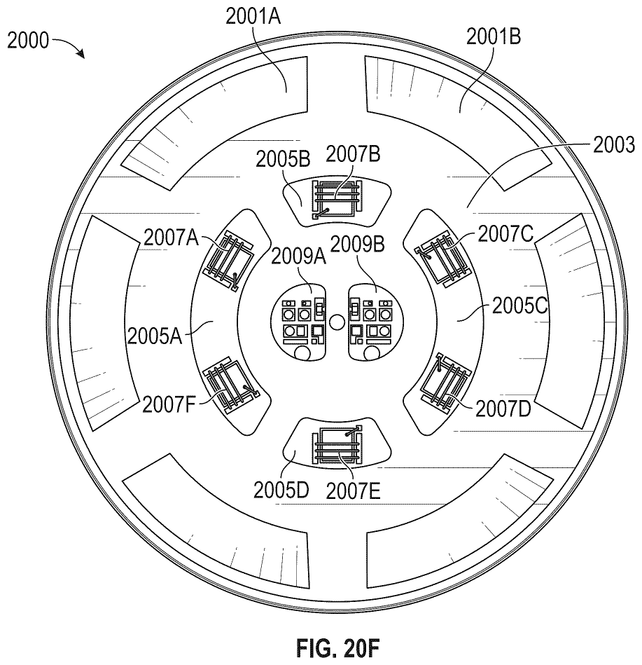

FIG. 20A is a front view of an example sensor assembly 2000 which can include a frame 2003, electrodes 2001A, 2001B, emitter chambers 2009A, 2009B housing one or more emitters, and detector chamber 2005 housing detectors 2007A-2007F. The sensor assembly 2000, or components thereof, can include similar structural and/or operational features of any of the other example sensor assemblies, or components thereof, shown and/or described herein.

The detector chamber 2005 may be annular. The detector chamber 2005 may surround the emitter chambers 2009A, 2009B. Optical radiation that enters the detector chamber 2005 may travel through the detector chamber 2005 between the various detectors 2007A-2007F. For example, any of the detectors 2007A-2007F may detect optical radiation that was detected by any of the other detectors 2007A-2007F as the optical radiation travels through the detector chamber 2005. In some implementations, allowing optical radiation to travel among detectors 2007A-2007F within the detector chamber 2005 may reduce accuracy of physiological measurements.

Detector chamber 2005 can have an optical radiation detecting area between about 50 mm² and about 150 mm², between about 75 mm² and about 125 mm², between about 85 mm² and about 115 mm², between about 95 mm² and about 105 mm², or any value or range between any of these values or ranges or any value or range bounded by any combination of these values, although values or ranges outside these values or ranges can be used in some cases.

FIG. 20B is a front view of another example implementation of a sensor assembly 2000 which can include a frame 2003, electrodes 2001A, 2001B, emitter chambers 2009A, 2009B housing one or more emitters, detector chamber 2005A housing detectors 2007A-2007C, and detector chamber 2005B housing detectors 2007D-2007F. The sensor assembly 2000, or components thereof, can include similar structural and/or operational features of any of the other example sensor assemblies, or components thereof, shown and/or described herein.

The detector chambers 2005A, 2005B may be semi-annular and may surround the emitter chambers 2009A-2009B. The frame 2003 may inhibit optical radiation within detector chamber 2005A from entering detector chamber 2005B, and vice versa. Accordingly, having a plurality of detector chambers 2005A-2005B may improve an accuracy of physiological measurements at least by controlling the optical radiation exposed to the detectors 2007A-2007F at least by inhibiting optical radiation from travelling among the detectors 2007A-2007F between the detector chambers 2005A-2005B.

In the example implementation shown in FIG. 20B, detector chamber 2005A is symmetrical with detector chamber 2005B about a first line and the emitter chamber 2009A is symmetrical with emitter chamber 2009B about a second line and the first and second lines are orthogonal. Detector 2007B and/or detector 2007E may be bisected by a line about which emitter chamber 2009A is symmetrical with emitter chamber 2009B.

Any of detector chambers 2005A-2005B can have an optical radiation detecting area between about 25 mm² and about 75 mm², between about 30 mm² and about 65 mm², between about 35 mm² and about 60 mm², between about 40 mm² and about 50 mm², or any value or range between any of these values or ranges or any value or range bounded by any combination of these values, although values or ranges outside these values or ranges can be used in some cases.

FIG. 20C is a front view of another example implementation of a sensor assembly 2000 which can include a frame 2003, electrodes 2001A, 2001B, emitter chambers 2009A, 2009B housing one or more emitters, detector chamber 2005A housing detectors 2007A, 2007B, 2007F, and detector chamber 2005B housing detectors 2007C-2007E. The sensor assembly 2000, or components thereof, can include similar structural and/or operational features of any of the other example sensor assemblies, or components thereof, shown and/or described herein.

The detector chambers 2005A, 2005B may be semi-annular and may surround the emitter chambers 2009A-2009B. The frame 2003 may inhibit optical radiation within detector chamber 2005A from entering detector chamber 2005B, and vice versa. Accordingly, having a plurality of detector chambers 2005A-2005B may improve an accuracy of physiological measurements at least by controlling the optical radiation exposed to the detectors 2007A-2007F at least by inhibiting optical radiation from travelling among the detectors 2007A-2007F between the detector chambers 2005A-2005B.

In the example implementation shown in FIG. 20C, detector chamber 2005A is symmetrical with detector chamber 2005B about a line and the emitter chamber 2009A is orthogonal with emitter chamber 2009B about that same line.

FIG. 20D is a front view of another example implementation of a sensor assembly 2000 which can include a frame 2003, electrodes 2001A, 2001B, emitter chambers 2009A, 2009B housing one or more emitters, detector chamber 2005A housing detectors 2007A, 2007E, 2007F, and detector chamber 2005B housing detectors 2007B-2007D. The sensor assembly 2000, or components thereof, can include similar structural and/or operational features of any of the other example sensor assemblies, or components thereof, shown and/or described herein.

The detector chambers 2005A, 2005B may be semi-annular and may surround the emitter chambers 2009A-2009B. The frame 2003 may inhibit optical radiation within detector chamber 2005A from entering detector chamber 2005B, and vice versa. Accordingly, having a plurality of detector chambers 2005A-2005B may improve an accuracy of physiological measurements at least by controlling the optical radiation exposed to the detectors 2007A-2007F at least by inhibiting optical radiation from travelling among the detectors 2007A-2007F between the detector chambers 2005A-2005B.

In the example implementation shown in FIG. 20D, detector chamber 2005A is symmetrical with detector chamber 2005B about a first line and the emitter chamber 2009A is orthogonal with emitter chamber 2009B about a second line and the first and second lines are non-orthogonal and non-parallel.

FIG. 20E is a front view of another example implementation of a sensor assembly 2000 which can include a frame 2003, electrodes 2001A, 2001B, emitter chambers 2009A, 2009B housing one or more emitters, detector chamber 2005A housing detectors 2007A, 2007F, detector chamber 2005B housing detectors 2007B-2007C, and detector chamber 2005C housing detectors 2007D-2007E. The sensor assembly 2000, or components thereof, can include similar structural and/or operational features of any of the other example sensor assemblies, or components thereof, shown and/or described herein.

The detector chambers 2005A-2005C may be semi-annular and may surround the emitter chambers 2009A-2009B. The frame 2003 may inhibit optical radiation within any of the detector chambers 2005A-2005C from entering any of the other detector chambers 2005A-2005C. Accordingly, having a plurality of detector chambers 2005A-2005C may improve an accuracy of physiological measurements at least by controlling the optical radiation exposed to the detectors 2007A-2007F at least by inhibiting optical radiation from travelling among the detectors 2007A-2007F between the detector chambers 2005A-2005C.

Any of detector chambers 2005A-2005C can have an optical radiation detecting area between about 10 mm² and about 45 mm², between about 15 mm² and about 40 mm², between about 20 mm² and about 35 mm², between about 23 mm² and about 32 mm², or any value or range between any of these values or ranges or any value or range bounded by any combination of these values, although values or ranges outside these values or ranges can be used in some cases.

FIG. 20F is a front view of another example implementation of a sensor assembly 2000 which can include a frame 2003, electrodes 2001A, 2001B, emitter chambers 2009A, 2009B housing one or more emitters, detector chamber 2005A housing detectors 2007A, 2007F, detector chamber 2005B housing detectors 2007B, detector chamber 2005C housing detectors 2007C-2007D, detector chamber 2005D housing detectors 2007E. The sensor assembly 2000, or components thereof, can include similar structural and/or operational features of any of the other example sensor assemblies, or components thereof, shown and/or described herein.

The detector chambers 2005A-2005D may surround the emitter chambers 2009A-2009B. The frame 2003 may inhibit optical radiation within any of the detector chambers 2005A-2005D from entering any of the other detector chambers 2005A-2005D. Accordingly, having a plurality of detector chambers 2005A-2005D may improve an accuracy of physiological measurements at least by controlling the optical radiation exposed to the detectors 2007A-2007F at least by inhibiting optical radiation from travelling among the detectors 2007A-2007F between the detector chambers 2005A-2005D.

A greater number of detector chambers (e.g., fewer detectors per chamber) may facilitate more accurate physiological measurements than fewer detector chambers (e.g., more detectors per chamber). For example, six detector chambers (e.g., one detector per chamber as shown in FIGS. 16A, 17A) may facilitate more accurate physiological measurements than five detector chambers which may in turn facilitate more accurate measurements than four detector chambers (e.g., one or two detectors per chamber as shown in FIG. 20F) which may in turn facilitate more accurate measurements than three detector chambers (e.g., two detectors per chamber as shown in FIG. 20E) which may in turn facilitate more accurate measurements than two detector chambers (e.g., three detectors per chamber as shown in FIGS. 20B-20D) which may in turn facilitate more accurate measurements than one detector chambers (e.g., six detectors per chamber as shown in FIG. 20A).

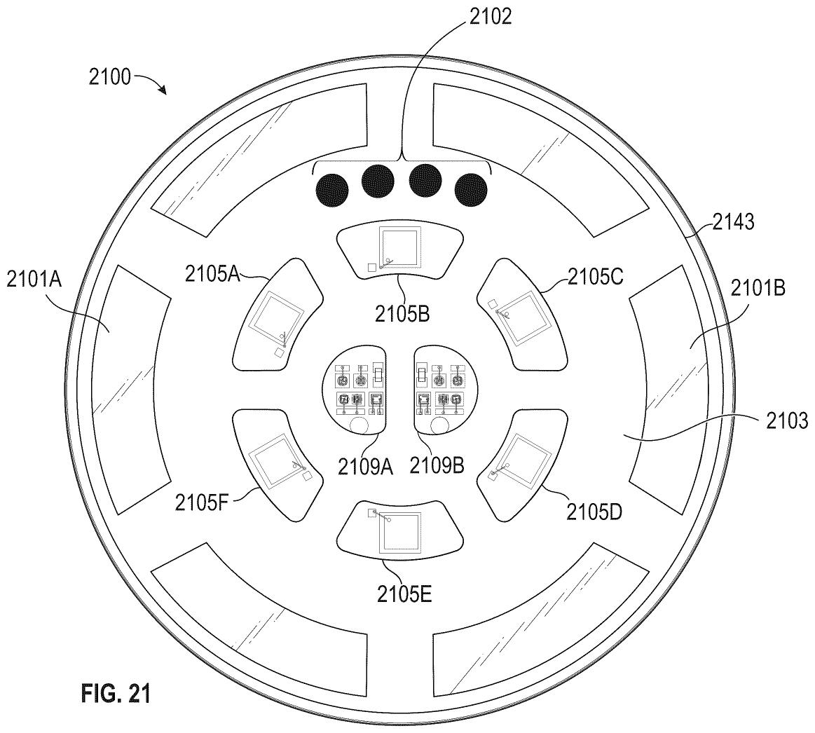

FIG. 21 is a front view of another example implementation of a sensor assembly 2100 which can include any of structural and/or operational features of any of the other example sensor assemblies shown and/or described herein. The sensor assembly 2100 can include one or more charging ports 2102. The charging ports 2102 can be made of an electrically conductive material such as metal. The charging ports 2102 can have a circular cross section. The charging ports 2102 can contact one or more electrical components within the sensor assembly 2100, such as a substrate, an electrical bus, and/or a battery. The charging ports 2102 can conduct electrical energy to an interior of the sensor assembly 2100 to charge the sensor assembly 2100 and/or to charge an electrical device into which the sensor assembly 2100 is integrated such as a watch. The charging ports 2102 can be raised from a surface of the sensor assembly 2100. The charging ports 2102 can be exposed and can contact an electrical power supply such as corresponding charging ports on a charging device. In some implementations, the charging ports 2102 can be recessed into the sensor assembly 2100.

In this example, charging ports 2102 are positioned between the detector chambers 2105 and the perimeter 2143 and are thus positioned on the surface of the frame 2103 that is configured to contact the user's skin when worn. In this example, charging ports 2102 are also positioned between the detector chambers 2105 and the electrodes 2101. In some implementations, charging ports 2102 may be positioned between the electrodes 2101 and perimeter 2143. In some implementations, charging ports 2102 may be positioned between the detector chambers 2105 and emitter chambers 2109. In some implementations, charging ports 2102 may be positioned on a surface of the frame 2103 that is not configured to contact the user's skin when worn.

The sensor assembly 2100 can include four charging ports, as shown. In some implementations, the sensor assembly 2100 can have one, two, three, or more than four charging ports. Charging ports 2102 may be positioned on the sensor assembly 2100 at mirror image locations from each other, such as across a vertical line with respect to the page or in some cases across a horizontal line with respect to the page. In some implementations, charging ports 2102 may be positioned on the sensor assembly 2100 diagonally from each other with respect to the page.

The sensor assembly 2100 can include one or more magnets which can be positioned within an interior region of the sensor assembly 2100 and/or may be embedded within a surface of the sensor assembly 2100. The magnets may attract corresponding magnets in a separate device that can be used to charge a wearable device into which the sensor assembly 2100 is integrated. The magnets may help to align the charging ports 2102 with corresponding charging ports on a separate charging device. The magnets may be placed adjacent to the charging ports 2102 or may be integrated within the charging ports 2102 or may be distanced from the charging ports 2102.

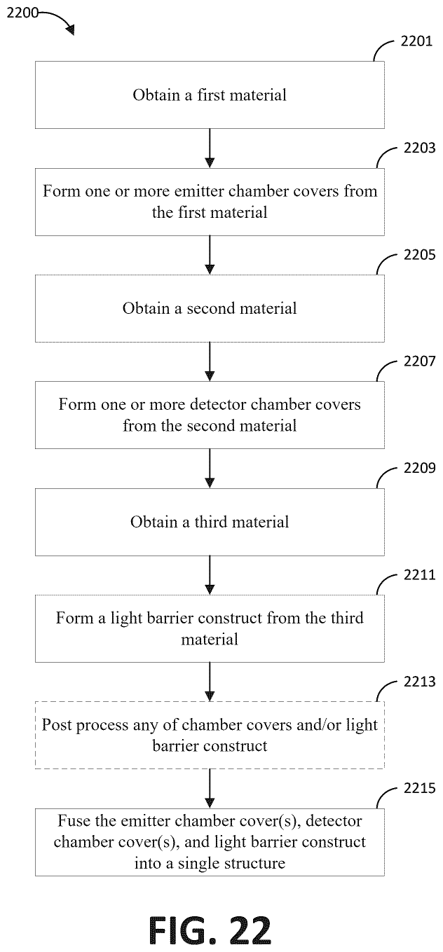

FIG. 22 illustrates a process 2200 of forming any of the above-described example sensor assemblies and components thereof, including emitter chamber covers, detector chamber covers, and frames/light barrier constructs, although a variety of other suitable methods may exist.

As shown in operational block 2201, the example method 2200 can include obtaining a first material. The first material can include glass (such as glass microspheres, glass nanospheres, transparent glass, etc.), sapphire, ceramic, polymer, etc. The glass material can be any glass. In some implementations, the glass can be fused silica glass, e.g., an amorphous silicon dioxide. In some implementations, the glass can be a high purity glass. In some implementations, the glass can include one or more dopants, one or more modifiers, one or more additional agents, including but not limited to, a dispersion agent and/or a phase-forming agent, and/or one or more additives, for example, to form various types of glasses and/or properties.

The first material can include glass particles that can have an average particle size (e.g., an average diameter or an average largest dimension) or a median particle size (e.g., a median diameter or a median largest dimension) in the nanometer range. For example, the glass particles can have an average particle size (e.g., an average diameter or an average largest dimension) or a median particle size (e.g., a median diameter or a median largest dimension) in the range from 5 nm to 400 nm, e.g., from 10 nm to 200 nm or from 50 nm to 100 nm, etc. In various examples, the glass particles can have a bimodal or multimodal distribution to increase the density and reduce the amount of shrinkage. For example, the glass material can include glass particles having two or more size ranges. As an example, the glass material can further include additional glass particles having an average particle size or a median particle size in the micrometer range in addition to the glass particles having an average particle size or a median particle size in the nanometer range. For implementation, the glass material can further include glass particles having an average particle size or a median particle size in the range of 2 μm to 50 μm in addition to the glass particles having an average particle size or a median particle size in the range of 5 nm to 400 nm.

As shown in operational block 2203, the example method 2200 can include forming one or more emitter chamber covers, such as any of the emitter chamber covers disclosed herein. Block 2203 can include forming one or a plurality of emitter chamber covers (for example, two emitter chamber covers). As described herein, one or more emitters can be arranged within an emitter chamber. An emitter chamber cover can be used to cover the emitter chamber, as described elsewhere herein.

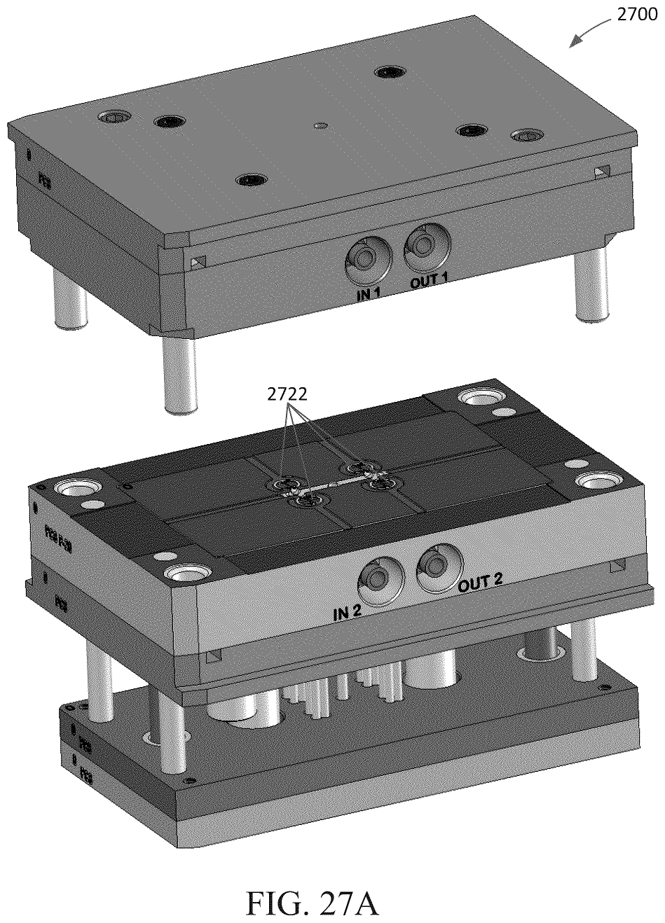

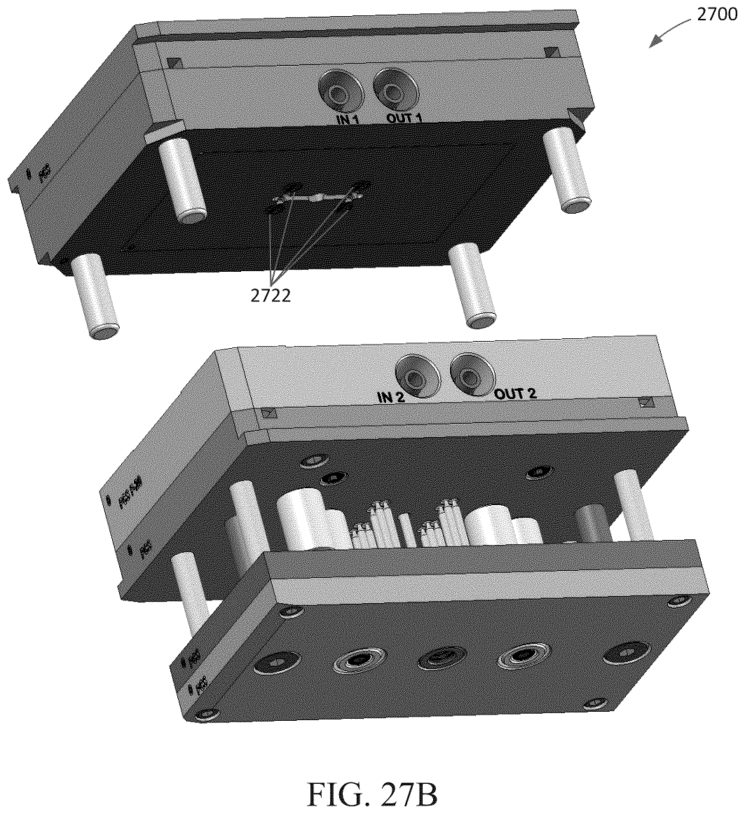

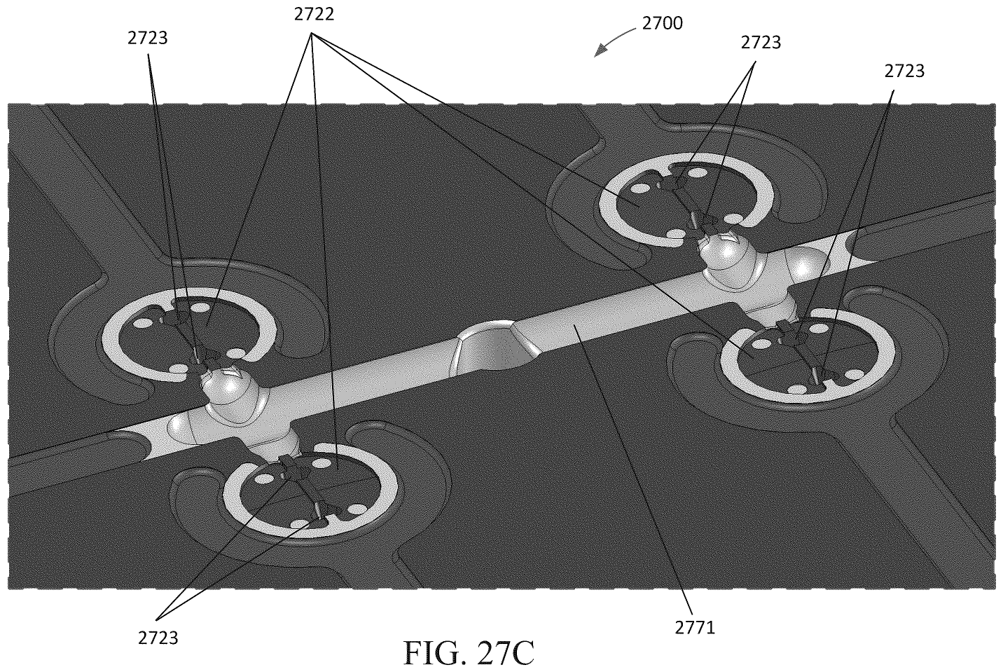

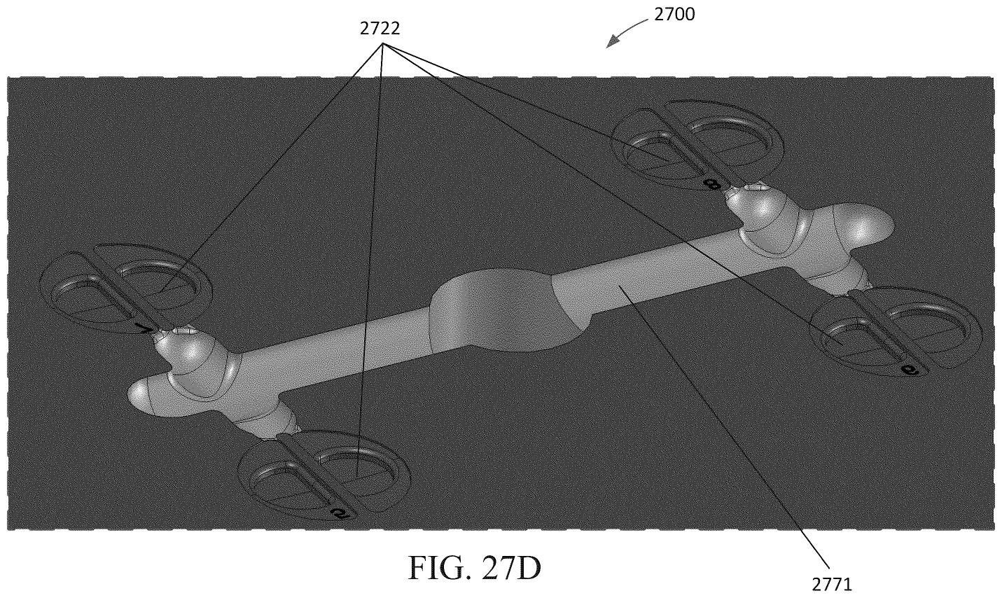

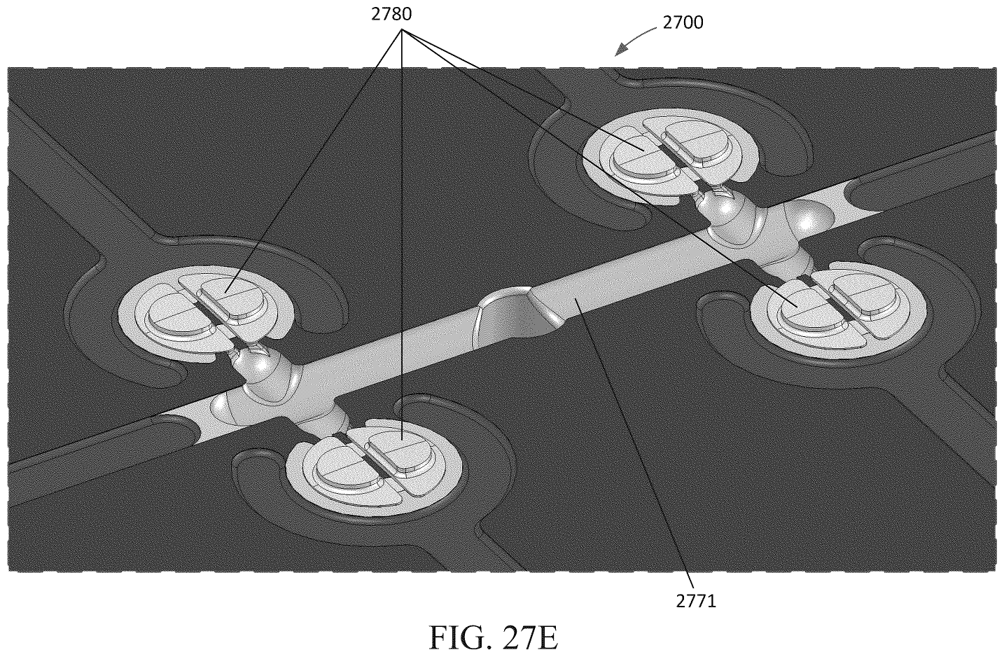

The one or more emitter chamber covers can be formed by molding, e.g., injection molding. Injection molding an emitter chamber cover using glass can include combining the first material into a binder. For example, glass particles can be dispersed in a binder. The content of the glass particles within the first material can be in the range of 50 to 75 parts per volume based on 100 parts per volume of the binder. The binder can be, for example, an organic thermoplastic binder. In various implementations, the organic thermoplastic binder can be solid at room temperature, and when heated, can soften and be molded into a desired shape. In various implementations, when heated, the binder can soften without any chemical reactions. Example organic thermoplastic binders, include but are not limited to, polyesters, polycarbonates, polyolefins, poly(vinyl butyral), etc. Example binders can have melting temperature in the range from 50° C. to 200° C., e.g., from 100° C. to 150° C. In various examples, the first material and binder can be placed in a mold and heated to form the desired shape (for example, to form any of the emitter chamber covers, detector chamber covers, and/or light barrier constructs disclosed herein). Such mold can be, for example, the mold described and/or shown in FIGS. 27A-27E. In various implementations, the composition can be heated to a temperature in the range of 40° C. to 400° C., e.g., in the range of 40° C. to 200° C., in the range of 40° C. to 150° C., etc.

As shown in operational block 2205, the example method 2200 can also include obtaining a second material. The second material can comprise any of the same characteristics, properties, and/or materials of the first material and in some implementations may be the same as the first material.

As shown in operational block 2207, the example method 2200 can also include forming one or more detector chamber covers. Block 2207 can include forming one or a plurality of detector chamber covers (for example, one, two, three, four, five, or six or more detector chamber covers). As described herein, one or more detectors can be arranged within a detector chamber (for example, of a sensor assembly of a wearable health monitoring device such as any of those disclosed herein). A detector chamber cover can be used to cover the detector chamber, as also described elsewhere herein. The one or more detector chamber covers can be formed by molding, e.g., injection molding. Injection molding a detector chamber cover using glass can include combining the second material into a binder as described at block 2203.

As shown in operational block 2209, the example method 2200 can also include obtaining a third material. The third material can comprise any of the same characteristics, properties, and/or materials of the first material and/or second material. The third material can include glass (such as glass microspheres, glass nanospheres, opaque glass, colored glass, etc.), sapphire, ceramic, polymer, etc. The third material can include one or more pigments such as an optically absorbent pigment. The pigment can provide color to the third material. The pigment may be black. The pigment can provide opacity to the third material and/or may render the third material opaque so as to inhibit transmission of optical radiation (e.g., light) through the third material, which can advantageously allow a light barrier construct (formed from the third material) to better optically isolate emitter chamber cover(s) (which can surround emitter(s)) from detector chamber cover(s) (which can surround detector(s)). The pigment can be evenly dispersed throughout the third material. The pigment can be infused with the third material. The pigment can be added to the third material after debinding described at block 2213, or in some cases, prior to injection molding.

As shown in operational block 2211, the example method 2200 can also include forming a light barrier construct. In various implementations, the light barrier construct can be constructed over one or more emitter chamber covers and over one or more detector chamber covers, for example, such that walls of the light barrier construct can separate the emitter chamber cover(s) from the detector chamber cover(s). In some examples, the light barrier construct can include walls that separate individual ones of a plurality of emitter chamber covers, as described elsewhere herein. In some examples, the light barrier construct can include walls that separate individual ones of a plurality of detector chamber covers. As described herein, the light barrier construct can include a plurality of walls that define one or more or a plurality of emitter chambers and one or more or a plurality of detector chambers. In some implementations, block 2211 comprises forming the light barrier construct such that: each of a plurality of emitter chambers are separated from one another; each of a plurality of detector chambers are separated from one another; and/or each of a plurality of emitter chambers are separated from the plurality of detector chambers. Blocks 2203 and/or block 2207 occur prior to, at the same time as, or after, block 2211. Block 2203 can occur prior to, at the same time as, or after block 2207.

The light barrier construct can be formed by molding, e.g., injection molding. The light barrier construct can be overmolded on and/or around the one or more emitter chamber covers and one or more detector chamber covers, in various examples, after forming the emitter chamber covers and detector chamber covers.

At optional block 2213, the method 2200 can optionally include post processing any of the emitter chamber covers, detector chamber covers, and/or light barrier construct. Post processing can include removing any excess material from a molded part, or the molded part can be ground and polished. Removing excess material can include removing the binder and any other remaining agents using a debinding process. The debinding process can include thermal treatment, chemical reaction, reduced pressure, and/or solvent extraction. As an example, the molded part (e.g., the light barrier construct overmolded over one or more emitter chamber covers and one or more detector chamber covers) can be immersed in a solvent for solvent extraction. The part can be heated, e.g., in a range from 150° C. to 600° C. and/or placed in sub atmospheric pressure. The debinding process can be based on the type of binder. The emitter chamber covers and/or detector chamber covers can undergo debinding individually prior to being overmolded with the light barrier construct. In some cases, emitter chamber covers, detector chamber covers, and light barrier construct can undergo debinding together after having been overmolded. Post processing can include adding pigment to the third material (e.g., after debinding) as described at block 2209. Pigment may not be added to the first and second materials.

At block 2215, the method 2200 can include fusing the emitter chamber covers, the detector chamber covers, and the light barrier construct into a single, unitary structure. Fusing can cause chemical bonds to form between the emitter chamber covers, the detector chamber covers, and the light barrier construct such that they become a single, contiguous mass. Such a unitary mass comprising bonded materials (e.g., first, second, and third materials) can have few or no discontinuities between the various materials which can provide numerous benefits including: 1) improving the overall look and/or feel of the structure thus enhancing user experience; and 2) reducing the amount of discontinuities that exist between the various materials along which light could travel (e.g., light piping). Fusing can include sintering. The particular sintering process used can be based on the materials. In some implementations, the light chamber covers and/or detector chamber covers may not be sintered until they have been overmolded with the light barrier construct such that the emitter chamber covers, detector chamber covers, and the light barrier construct are sintered together at the same time. In various examples, the sintering process can include heating at a temperature in the range of 700° C. to 1500° C. In some implementations, a pre-sintering process can be used at a temperature in the range of 400° C. to 700° C. In some implementations in which method 2200 includes both debinding at block 2213 and sintering at block 2215, said sintering involves exposing the emitter chamber cover(s), detector chamber cover(s), and light barrier construct to a higher temperature than a temperature utilized for said debinding. After sintering, the part can be cooled to room temperature. Sintering can cause the emitter chamber covers, detector chamber covers, and the light barrier construct to shrink. Accordingly, the emitter chamber covers, detector chamber covers, and the light barrier construct can be formed with molds that are larger than the desired final size to account for shrinking during sintering. For example, each part can be molded to be 5%-30% larger than the final size after sintering, or 5%-20% larger, 5%-15% larger, 5%-10% larger, 10%-30% larger, 15%-30% larger, 20%-30% larger, etc.

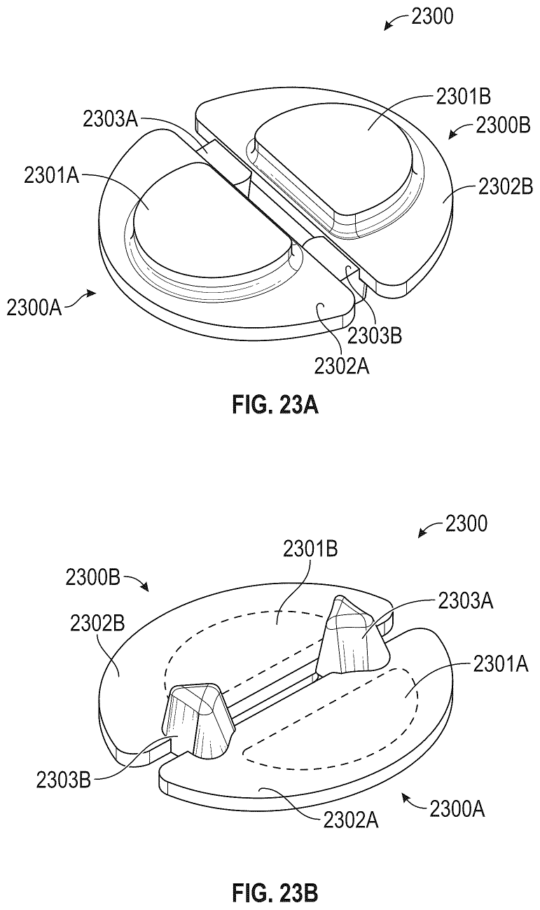

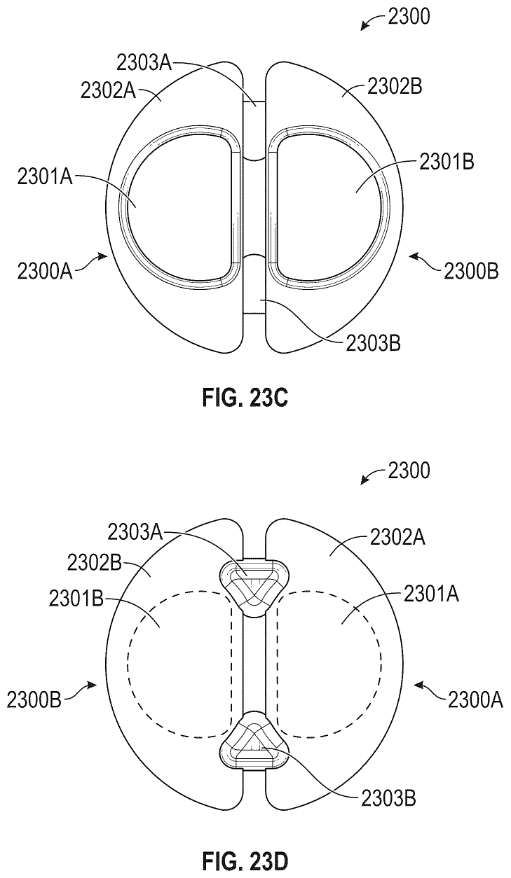

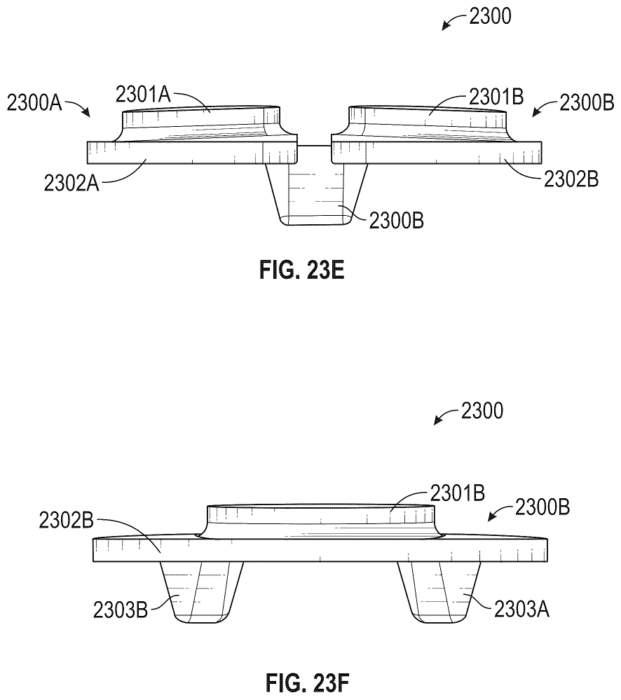

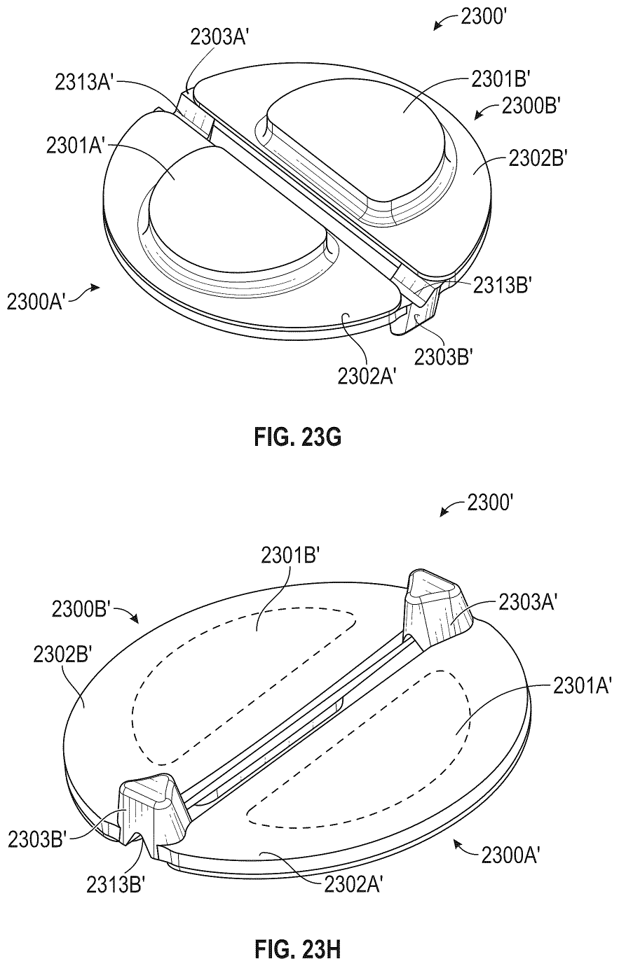

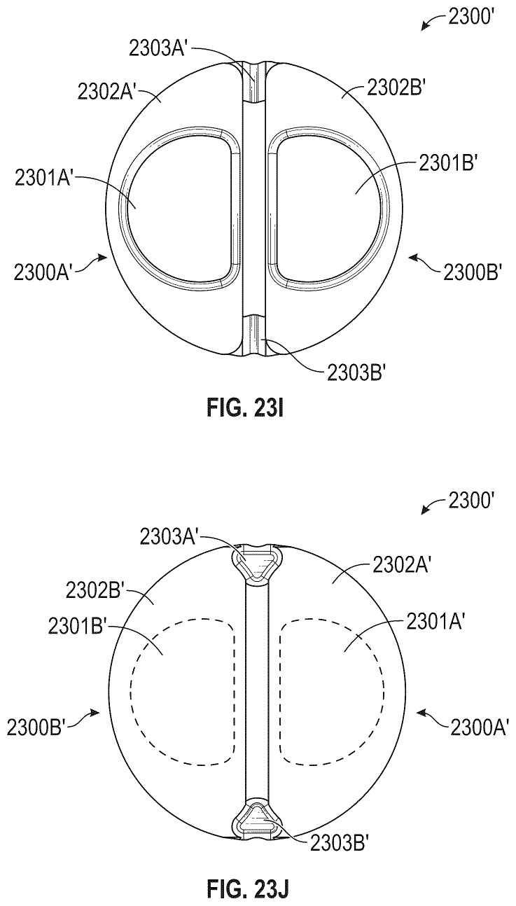

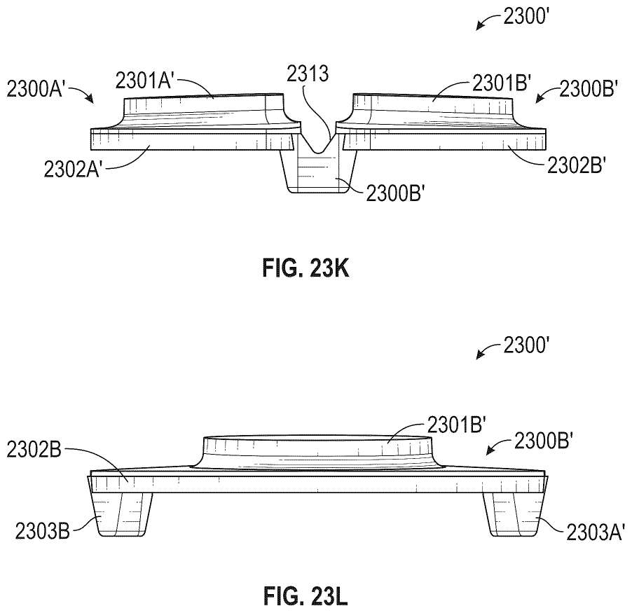

FIGS. 23A-23F illustrate various views of example emitter chamber covers 2300A, 2300B. Emitter chamber cover 2300A includes first portion 2301A and second portion 2302A. Emitter chamber cover 2300B includes first portion 2301B and second portion 2302B. First portions 2301A, 2301B may be referred to as “window portions”, “windows”, “lens portions”, or “lenses”. Second portions 2302A, 2302B may be referred to as “support portions” or “supports”. Emitter chamber covers 2300A, 2300B are optically transmissive (e.g., transparent) to allow optical radiation to pass therethrough. This example shows two emitter chamber covers 2200A-2300B. In some implementations, there may be one or more than two emitter chamber covers, depending on the number of emitter chambers according to any of the example sensor assembly implementations shown and/or described herein. Emitter chamber covers 2300A, 2300B can form a single piece component 2300 in some implementations. In some of such implementations, such single piece component 2300 can be integrally formed with and/or fused with light barrier construct 2503 (also referred to as “frame 2503” herein). In some of such implementations, portions of the emitter chamber covers 2300A, 2300B can be embedded within portions of the light barrier construct 2503.

Emitter chamber cover 2300A is connected to emitter chamber cover 2300B by one or more bridging portions 2303. In the example shown, two bridging portions 2303A, 2303B connect emitter chamber cover 2300A to emitter chamber cover 2300B. Bridging portions 2303A, 2303B connect to second portions 2302A, 2302B and extend away from second portions 2302A, 2302B. Bridging portions 2303A, 2303B can secure the emitter chamber covers 2300A, 2300B within the light barrier construct. Emitter chamber covers 2300A, 2300B, and bridging portions 2303A, 2303B may all be formed of the same material such as during an injection molding process. Accordingly, emitter chamber covers 2300A, 2300B and bridging portions 2303A, 2303B may form a single integrated structure all manufactured with the same process and/or materials at the same time, which can facilitate manufacturability. Accordingly, bridging portions 2303A, 2303B may be formed of an optically transmissive material. In this implementation, bridging portions 2303A, 2303B are closer to respective first portions 2301 than to the edge of respective second portions 2302. In some implementations, such as shown in FIG. 23J, bridging portions 2303A′, 2303B′ are closer to the edge of respective second portions 2302′ than to respective first portions 2301′. Distancing bridging portions 2303 from first portions 2301 can help to inhibit light piping.

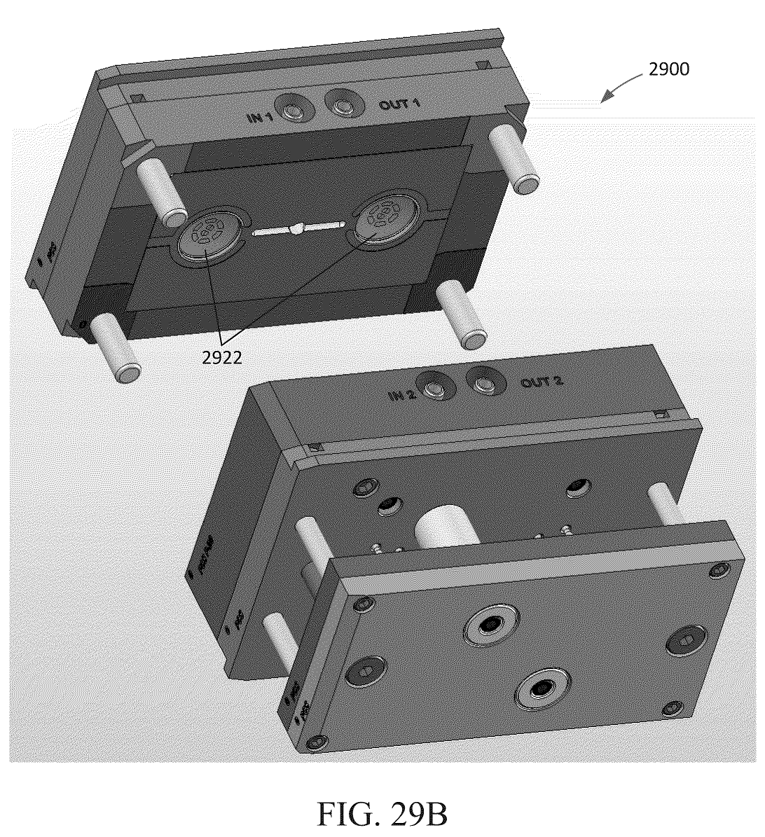

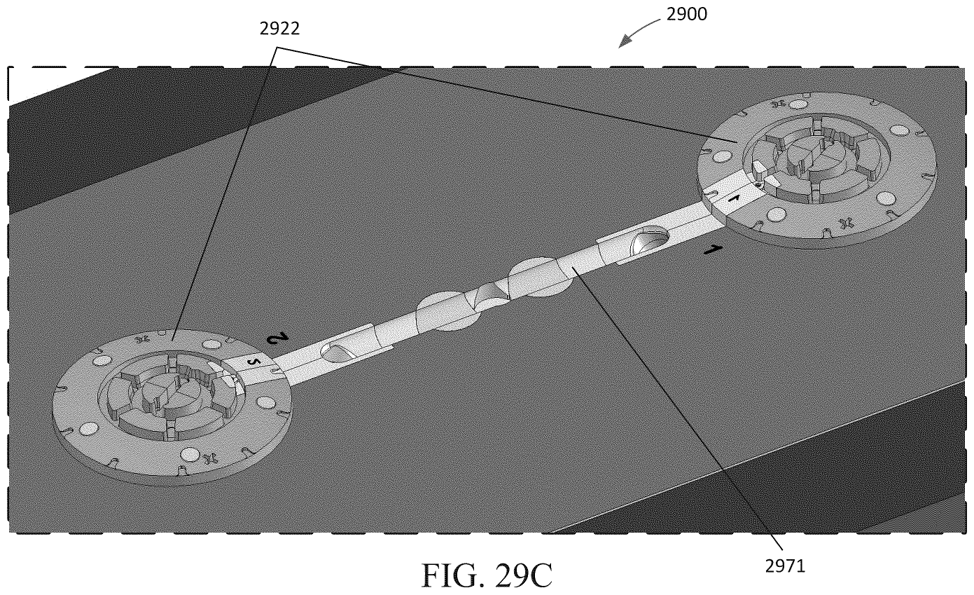

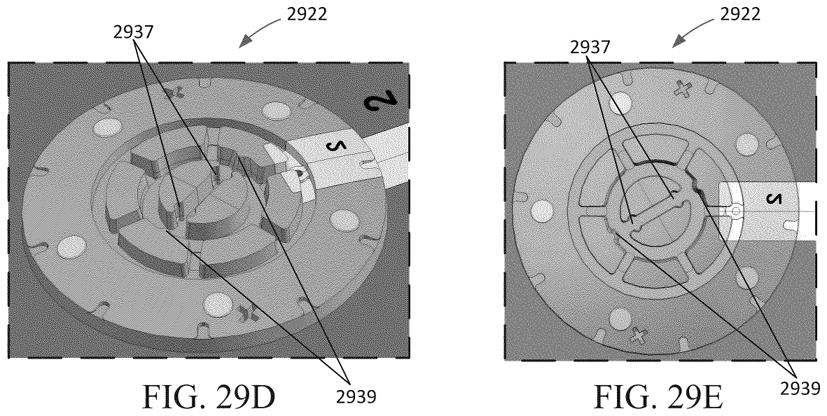

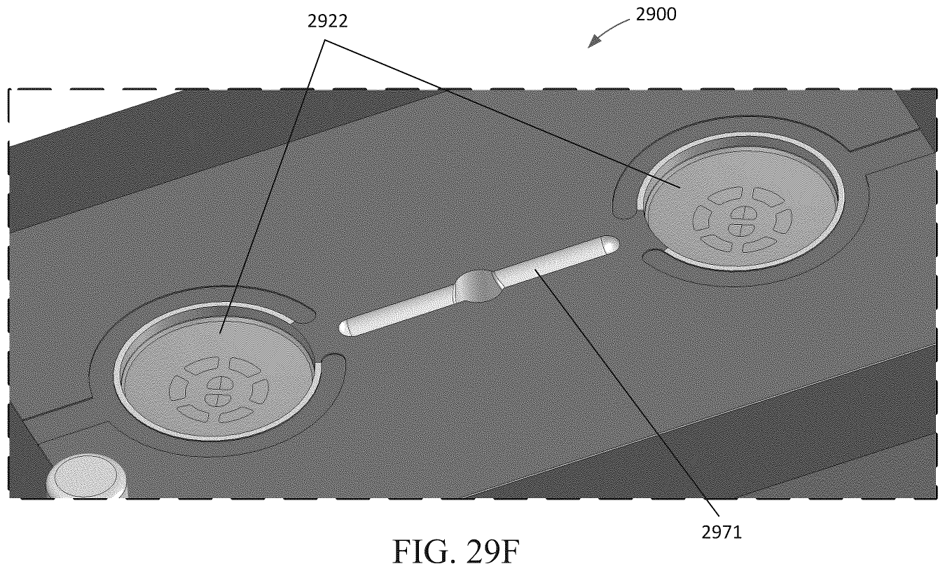

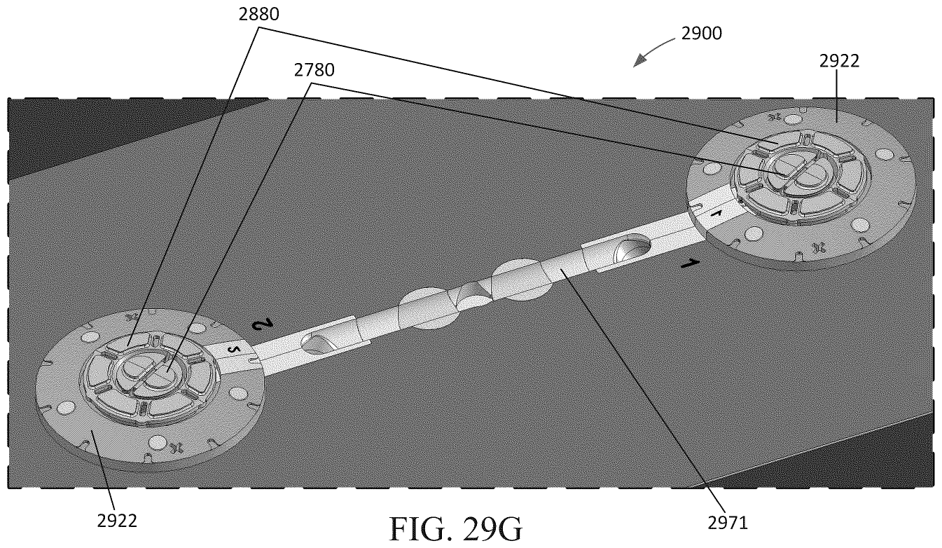

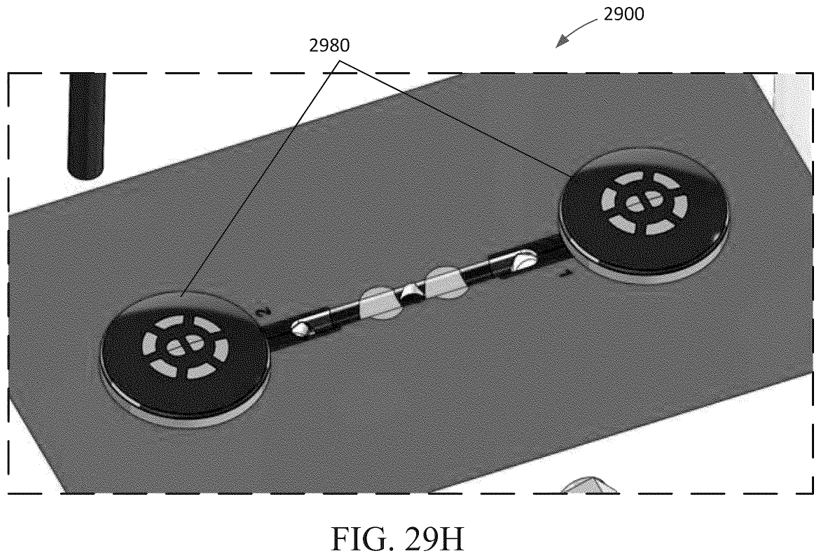

Bridging portions 2303 can help align the emitter chamber covers 2300A, 2300B within a mold when being overmolded with a frame. Bridging portions 2303 are tapered with the widest portions being connected to the emitter chamber covers 2300A, 2300B and the smaller portions being away from the emitter chamber covers 2300A, 2300B. The tapered shape of the bridging portions 2303 can facilitate placing the bridging portions 2303 within receptacles of a mold and properly aligning the emitter chamber covers 2300A, 2300B within the mold relative to other components as shown and/or described in FIGS. 29D-29E.

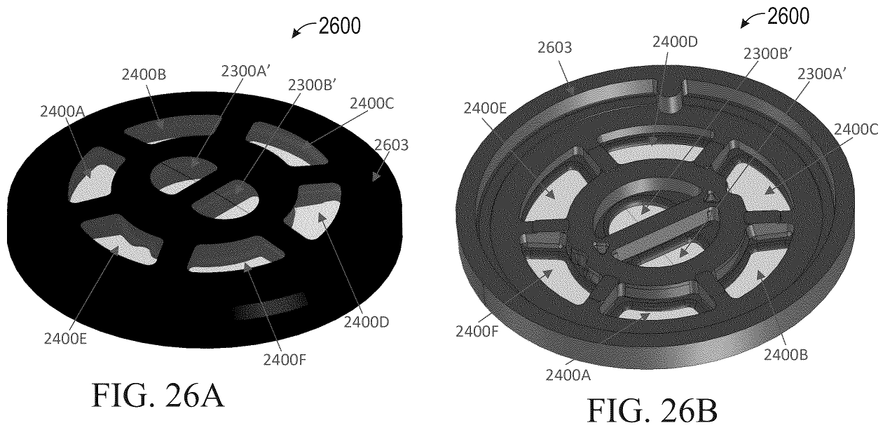

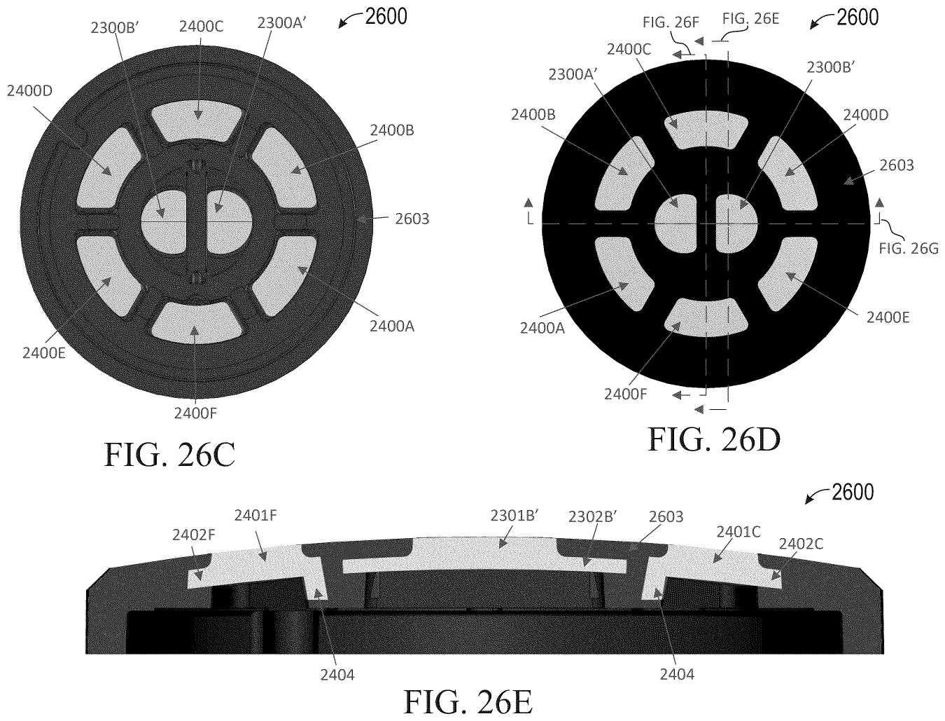

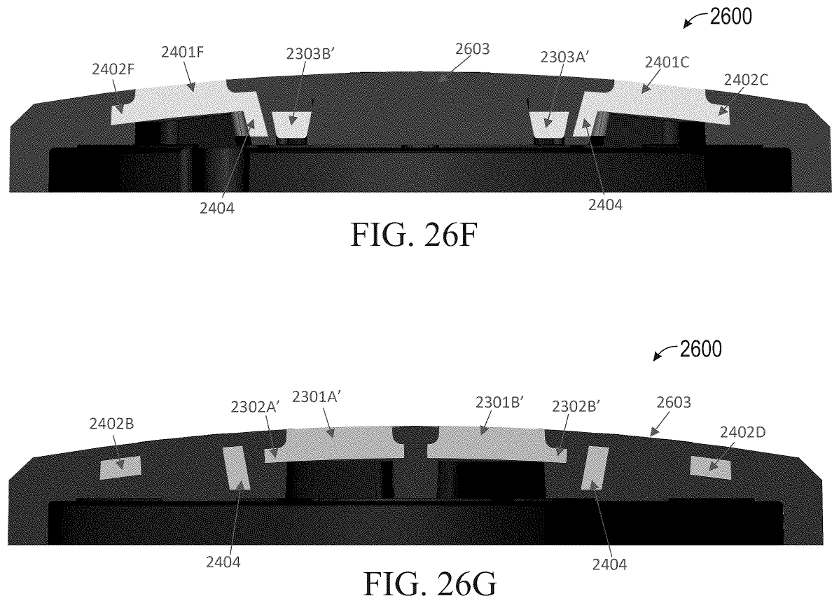

First portion 2301A protrudes from second portion 2302A. First portion 2301A can fit within an opening in a light barrier construct (e.g., a window). First portion 2301A can be sized and shaped to fit within any of the emitter chamber openings (e.g., windows) shown and/or described herein. Optical radiation can pass through first portion 2301A. Second portion 2302A extends around first portion 2301A. Second portion 2302A can be secured within the light barrier construct as shown and/or described in greater detail in FIGS. 25E-25G and/or FIGS. 26E-26G. First portion 2301B can have similar structural and/or operational features as first portion 2301A. Second portion 2302B can have similar structural and/or operational features as second portion 2302A.

FIGS. 23G-23L illustrate various views of example emitter chamber covers 2300A′, 2300B′ including first portions 2301A′, 2301B′ and second portions 2302A′, 2302B′. Emitter chamber covers 2300A′, 2300B′ are connected by bridging portions 2303A′, 2303B′. Emitter chamber covers 2300A′, 2300B′ can have similar structural and/or operational features as emitter chamber covers 2300A, 2300B. Emitter chamber covers 2300A′, 2300B′ may be formed of the same material as emitter chamber covers 2300A, 2300B (e.g., optically transmissive glass). Emitter chamber covers 2300A′, 2300B′ can form a single piece component 2300′ in some implementations. In some of such implementations, such single piece component 2300′ can be integrally formed with and/or fused with light barrier construct 2503 (also referred to as “frame 2503” herein). In some of such implementations, portions of the emitter chamber covers 2300A′, 2300B′ can be embedded within portions of the light barrier construct 2503.

Bridging portions 2303A′, 2303B′ include respective notches 2313A′, 2313B′. Notches 2313A′, 2313B′ reduce the amount of material (e.g., optically transmissive material) that connects emitter chamber cover 2300A′ to emitter chamber cover 2300B′, thus reducing the possibility of light piping between emitter chamber covers 2300A′, 2300B′. For example, notches 2313A′, 2313B′ may force at least some of the optical radiation travelling between emitter chamber covers 2300A′, 2300B′ through bridging portions 2303A′, 2303B′ to either travel along a curved path through bridging portions 2303A′, 2303B′ or to exit and re-enter bridging portions 2303A′, 2303B′, which can reduce the amount of optical radiation that completely traverses bridging portions 2303A′, 2303B′.

FIGS. 23B, 23D, 23H, and 23J are shown with broken lines indicating the outlines of first portions 2301A, 2301B, 2301A′, 2301B′. The broken lines are shown for illustrative purposes only. First and second emitter chamber covers 2300A, 2300B, 2300A′, 2300B′ may not include said broken lines or any other indication marking the outline of first portions 2301A, 2301B, 2301A′, 2301B′.

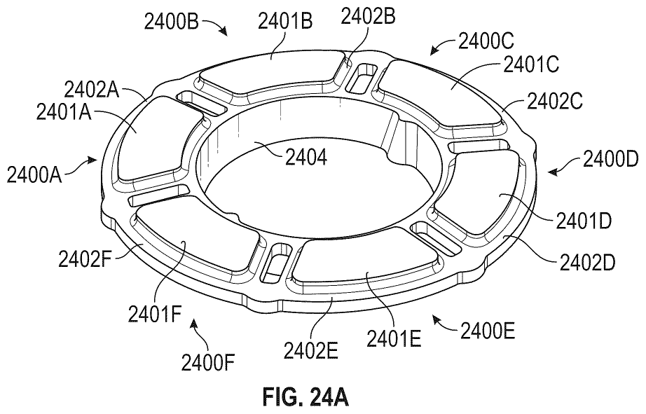

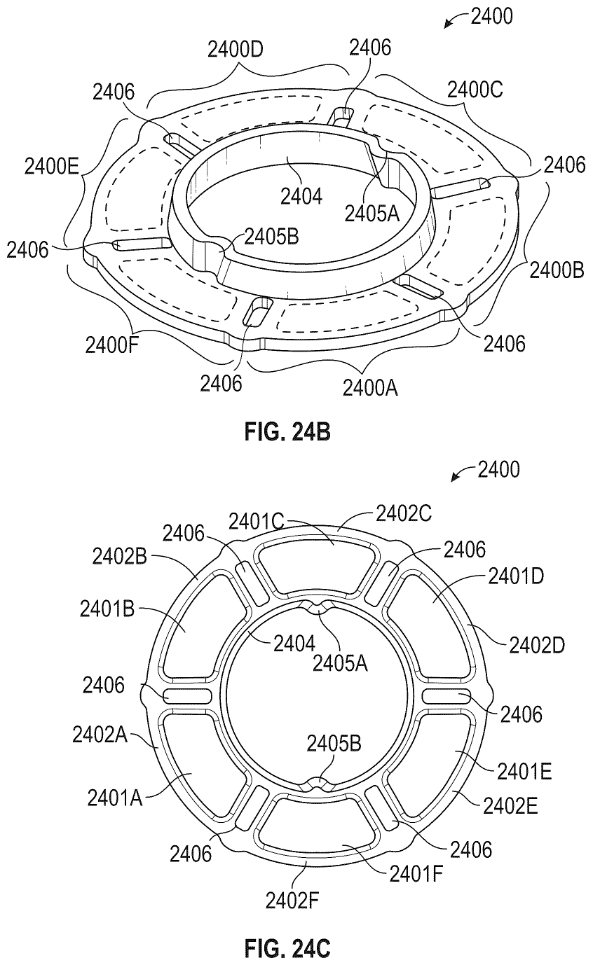

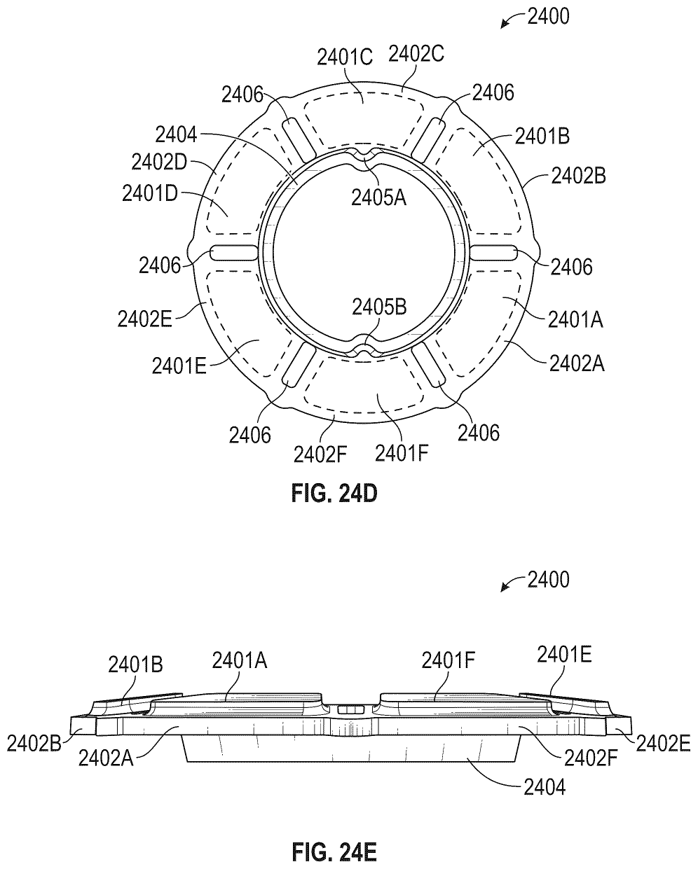

FIGS. 24A-24E illustrate various views of detector chamber covers 2400A-2400F. Each of the respective detector chamber covers 2400 can include a first portion 2401 and a second portion 2402. First portions 2401A-2401F may be referred to as “window portions”, “windows”, “lens portions”, or “lenses”. Second portions 2402A-2402F may be referred to as “support portions” or “supports”. This example shows six detector chamber covers 2400A-2400F. In some implementations, there may be fewer or more than six detector chamber covers, depending on the number of detector chambers according to any of the example sensor assembly implementations shown and/or described herein. Detector chamber covers 2400A-2400F can be arranged in a closed loop (e.g., an annulus) as shown here. Detector chamber covers 2400A-2400F can form a single piece component 2400 in some implementations. In some of such implementations, such single piece component 2400 can be integrally formed with and/or fused with light barrier construct 2503 (also referred to as “frame 2503” herein). In some of such implementations, portions of the detector chamber covers 2400A-2400F can be embedded within portions of the light barrier construct 2503.

Detector chamber covers 2400A, 2400F are optically transmissive (e.g., transparent) to allow optical radiation to pass therethrough. The detector chamber covers 2400A-2400F may be more transparent than the emitter chamber covers 2300A-2300B. Accordingly, emitter chamber covers 2300A, 2300B may diffuse optical radiation more than detector chamber covers 2400A-2400F. The detector chamber covers 2400A-2400F may include texture to provide diffusing properties.

Detector chamber covers 2400A-2400F are connected to each other. For example, detector chamber cover 2400B is connected to detector chamber cover 2400A and detector chamber cover 2400C. Second portions 2402 of adjacent detector chamber covers 2400 may connect with each other. Detector chamber covers 2400A-2400F (and even skirt wall 2404), including first portions 2401 and second portions 2402, may all be formed of the same material such as during an injection molding process. Accordingly, detector chamber covers 2400A-2400F may all form a single integrated structure all manufactured with the same process and/or materials at the same time, which can facilitate manufacturability. Accordingly, first portions 2401 and second portions 2402 may be formed of an optically transmissive material.

Openings 2406 extend through the second portions 2402 of the detector chamber covers 2400A-2400F. Openings 2406 are positioned between adjacent detector chamber covers 2400 such that each opening 2406 extends through adjacent second portions 2402 that are connected to each other. Openings 2406 can reduce light piping between adjacent detector chamber covers 2400 by reducing the amount of optically transmissive material by which adjacent detector chamber covers 2400 are connected.

First portions 2401 protrude from respective second portions 2402. First portions 2401 can fit within openings in a light barrier construct (e.g., a window). First portions 2401 can be sized and shaped to fit within any of the detector chamber openings (e.g., windows) shown and/or described herein. Optical radiation can pass through first portions 2401. Second portions 2402 extend around respective first portions 2401. Second portions 2402 can be secured within the light barrier construct as shown and/or described in greater detail in FIGS. 25E-25G and/or FIGS. 26E-26G. First portions 2401 can each have similar structural and/or operational features as each other. Second portions 2402 can each have similar structural and/or operational features as each other.

Skirt wall 2404 is connected to the detector chamber covers 2400A-2400F (e.g., to the second portions 2402). Skirt wall 2404 extends away from detector chamber covers 2400A-2400F. Skirt wall 2404 can be embedded within light barrier construct and can secure detector chamber covers 2400 to the light barrier construct. In this example, skirt wall 2404 is annular. Skirt wall 2404 can include one or more indentations 2405A, 2405B. Indentations 2405A, 2405B can facilitate orienting the detector chamber covers 2400A-2400F within a mold when being overmolded with a frame. Indentations 2405A, 2405B are tapered with the narrowest portions being connected to the detector chamber covers 2400 and the wider portions being away from the detector chamber covers 2400. The tapered shape of the indentations 2405 can facilitate placing the indentations 2405 over corresponding protrusions of a mold and properly aligning the detector chamber covers 2400A-2400F within the mold relative to other components as shown and/or described in FIGS. 29D-29E.

FIGS. 24B and 24D are shown with broken lines indicating the outlines of first portions 2401. The broken lines are shown for illustrative purposes only. The detector chamber covers 2400A-2400F may not include said broken lines or any other indication marking the outline of first portions 2401.

FIGS. 25A-25G illustrate various views of a light barrier construct 2503 (also referred to as a “frame” herein) overmolded with one or more emitter chamber covers 2300 and one or more detector chamber covers 2400. The emitter chamber covers 2300A, 2300B and detector chamber covers 2400A-2400F are shown with opacity in FIGS. 25A-26G for illustrative purposes only so that they may be visualized. However, emitter chamber covers 2300A, 2300B and detector chamber covers 2400A-2400F may be more transparent than illustrated here. Emitter chamber covers 2300A, 2300B (which can form a single piece component 2300 as described above), detector chamber covers 2400A-F (which can form a single piece component 2400 as described above), and frame 2503 can form a single piece component 2500. Such single piece component 2500 can be connected to other portions of any of the wearable health monitoring devices described herein (for example, a housing of such device) and can be arranged adjacent skin of a user when the device is in use. Such single piece components 2500 can form part of a sensor assembly of any of the wearable health monitoring devices described herein, and such sensor assembly can further include emitter(s) arranged within emitter chamber(s), detector(s) arranged within detector chamber(s), a circuit layer (for example, a printed circuit board), among other components.

The frame 2503 is overmolded on the emitter chamber covers 2300A, 2300B and detector chamber covers 2400A-2400F. The frame 2503, emitter chamber covers 2300A, 2300B, and detector chamber covers 2400A-2400F form a unitary structure such as a single contiguous mass. For example, the frame 2503, emitter chamber covers 2300A, 2300B, and detector chamber covers 2400A-2400F can be fused together such that chemical bonds form between them. The frame 2503, emitter chamber covers 2300, and detector chamber covers 2400A-2400F can be fused through a sintering process. For example, after injection molding the frame 2503, emitter chamber covers 2300A, B, and detector chamber covers 2400A-2400F, they can each be sintered together at the same time such they fuse together to form a single unitary structure.

The frame 2503 is optically opaque. The frame 2503 can comprise glass with an optically absorbent pigment mixed with the glass. The optically absorbent pigment can be distributed throughout the frame 2503 such that all portions of the frame 2503 (e.g., surface of the frame and interior of the frame) are optically opaque. The frame 2503 may have a uniform opacity due to the distribution of optically absorbent pigment throughout the frame. The glass of the frame 2503 may be colored by the optically absorbent pigment. The optically absorbent pigment may be black causing the frame 2503 to also be black. In some implementations, the optically absorbent pigment may be some other color, such as gray, brown, green, blue, purple, etc.

The emitter chamber covers 2300A, 2300B and detector chamber covers 2400A-2400F are optically transmissive. For example, they may be transparent and can allow optical radiation to pass through them. The emitter chamber covers 2300A, 2300B and detector chamber covers 2400A-2400F may be formed of the same material, such as glass. The emitter chamber covers 2300A, 2300B and detector chamber covers 2400A-2400F may not include an optically absorbent pigment. Thus, the unitary structure comprising the frame 2503, emitter chamber covers 2300A, 2300B, and detector chamber covers 2400A-2400F (e.g., that are fused together with chemical bonds forming a single contiguous mass) can thus comprise optically opaque portions and optically transmissive portions.

As shown in at least FIGS. 25E-25G, the first portions 2301 of the emitter chamber covers 2300 and the first portions 2401 of the detector chamber covers 2400A-2400F are exposed and can contact the skin of the user. The second portions 2302 of the emitter chamber covers 2300 and the second portions 2402 of the detector chamber covers 2400A-2400F are embedded within the frame 2503 and are not exposed and thus may not contact the skin of the user. The frame 2503 separates the first portions 2301 of the emitter chamber covers 2300A, 2300B and the first portions 2401 of the detector chamber covers 2400A-2400F. The emitter chamber covers 2300A, 2300B may not be connected to the detector chamber covers 2400A-2400F by any optically transmissive materials which can help to inhibit undesirable light piping cross-talk between emitters and detectors. The only material that connects the emitter chamber covers 2300A, 2300B to the detector chamber covers 2400A-2400F may be the optically opaque frame 2503. The skirt wall 2040 is embedded with the frame 2503 and surrounds the emitter chamber covers 2300A, 2300B.

FIGS. 26A-26G illustrate various views of a light barrier construct 2603 (also referred to as a “frame” herein) overmolded with one or more emitter chamber covers 2300A′, 2300B′ and one or more detector chamber covers 2400A-2400F. The emitter chamber covers 2300A′, 2300B′ and detector chamber covers 2400A-2400F are shown as transparent in FIGS. 26A-26D to illustrate their optical transmissivity. The emitter chamber covers 2300A′, 2300B′ and detector chamber covers 2400A-2400F are shown as having more opacity in FIGS. 26E-26G for illustrative purposes only. The frame 2603, emitter chamber covers 2300A′, 2300B′, and detector chamber covers 2400A-2400F can have similar structural and/or operational features (individually and/or collectively) as the frame 2503, emitter chamber covers 2300A, 2300B, and detector chamber covers 2400A-2400F shown and/or described in FIGS. 25A-25G. Frame 2603 may be formed of the same material(s) as frame 2503 and thus may be optically opaque. Frame 2603 may be overmolded on the emitter chamber covers 2300A′, 2300B′ and detector chamber covers 2400A-2400F. The frame 2603, emitter chamber covers 2300A′, 2300B′, and detector chamber covers 2400A-2400F can be fused together (e.g., via sintering) such that they form a single contiguous mass being chemically bonded together. Frame 2603 may be smaller than frame 2503 (e.g., smaller diameter). Emitter chamber covers 2300A′, 2300B′ (which can form a single piece component 2300′ as described above), detector chamber covers 2400A-F (which can form a single piece component 2400 as described above), and frame 2603 can form a single piece component 2600. Such single piece component 2600 can be connected to other portions of any of the wearable health monitoring devices described herein (for example, a housing of such device) and can be arranged adjacent skin of a user when the device is in use. Such single piece components 2600 can form part of a sensor assembly of any of the wearable health monitoring devices described herein, and such sensor assembly can further include emitter(s) arranged within emitter chamber(s), detector(s) arranged within detector chamber(s), a circuit layer (for example, a printed circuit board), among other components.

FIGS. 27A-27E illustrate various views of a mold assembly 2700 for emitter chamber covers. The mold assembly 2700 includes two halves that mate together to form molds 2722. The molds 2722 are openings between the two halves of the molds assembly 2700 into which material can be placed (e.g., injected) and assume the shape of the molds 2722. Material can be inserted into the molds 2722 via channel 2771. Material can be injected into molds in a viscous/liquid state such as when heated and can then harden into a more solid state as it cools within the molds 2722 and thus take the shape of the molds 2722. Various materials can be injected into the molds 2722 including glass, plastic, or any of the other materials shown and/or described herein. In this example, mold assembly 2700 includes four molds 2722 for creating four sets of emitter chamber covers 2780. Once the sets of emitter chamber covers 2780 have sufficiently hardened after having been injected molded, the sets of emitter chamber covers 2780 can be removed from the mold assembly 2700. In this example, each set of emitter chamber covers 2780 includes two emitter chamber covers that are integrally connected and formed from the same material at the same time during the same injection molding process. Molds 2722 can include cavities 2723 configured for forming bridging portions 2303A′, 2303B′ (see FIGS. 27C and 23H).

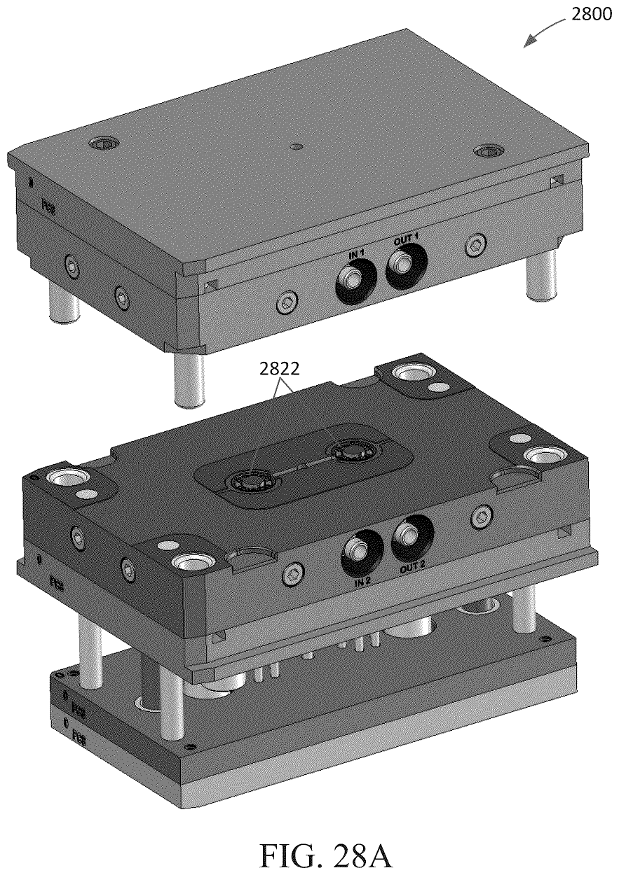

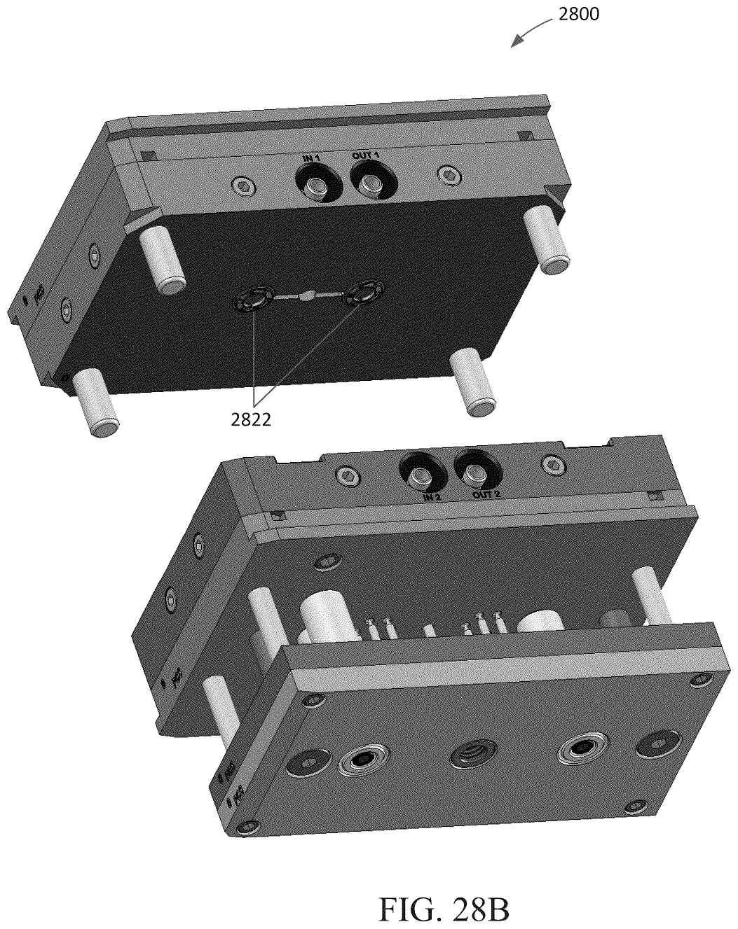

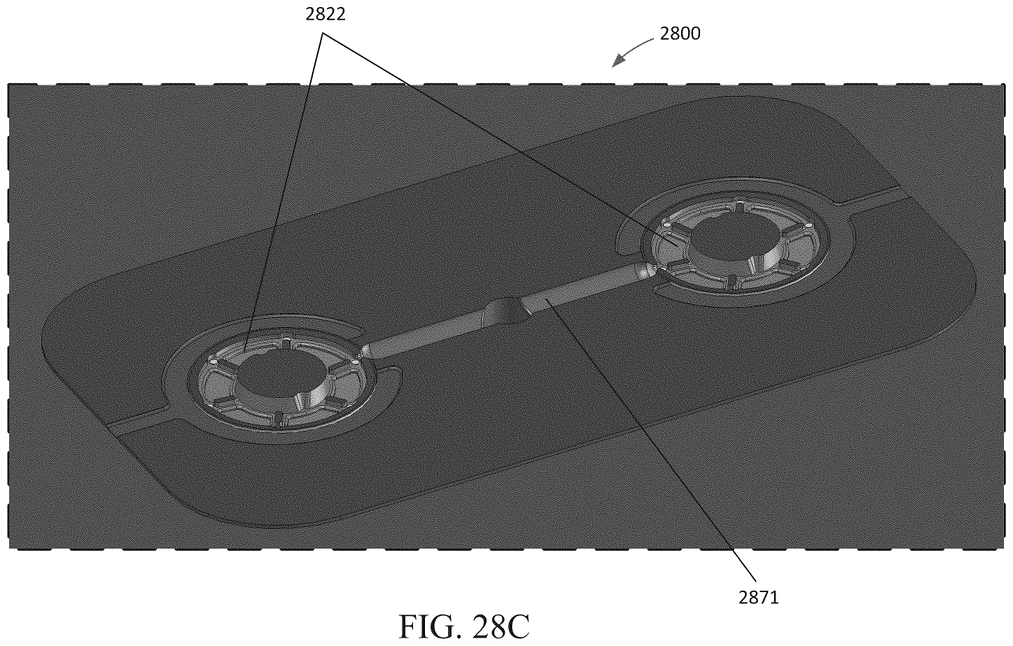

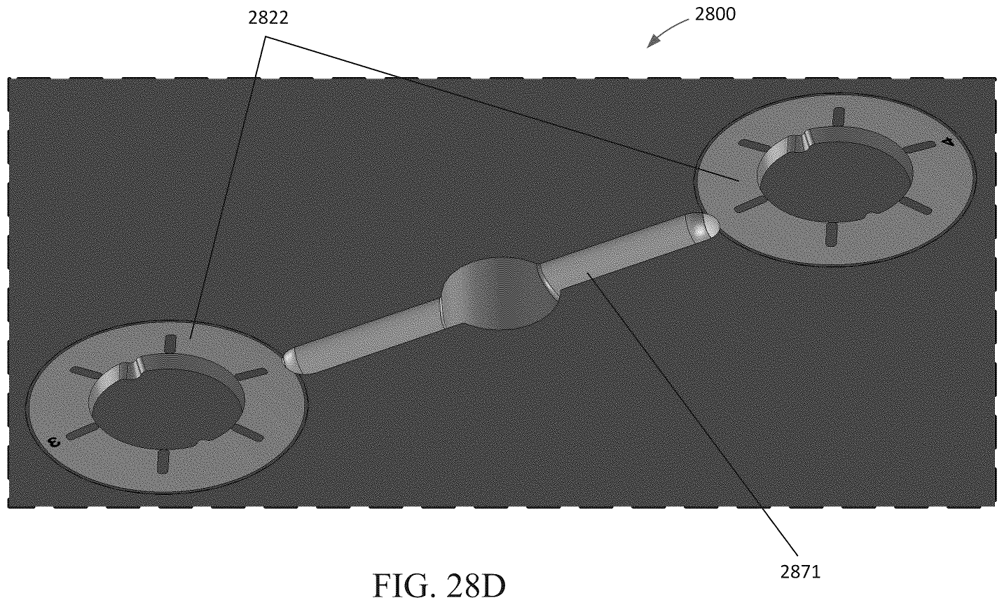

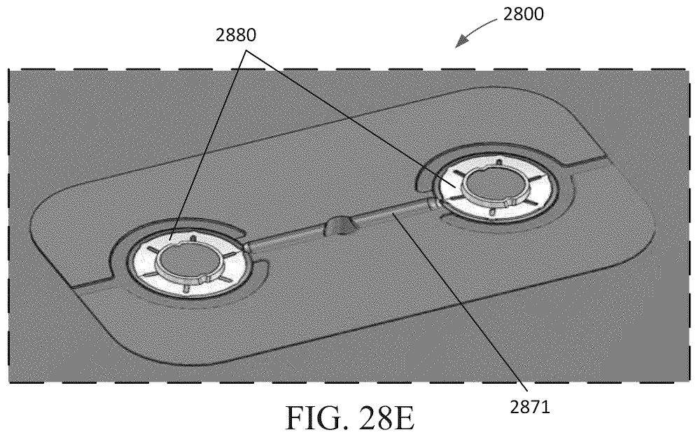

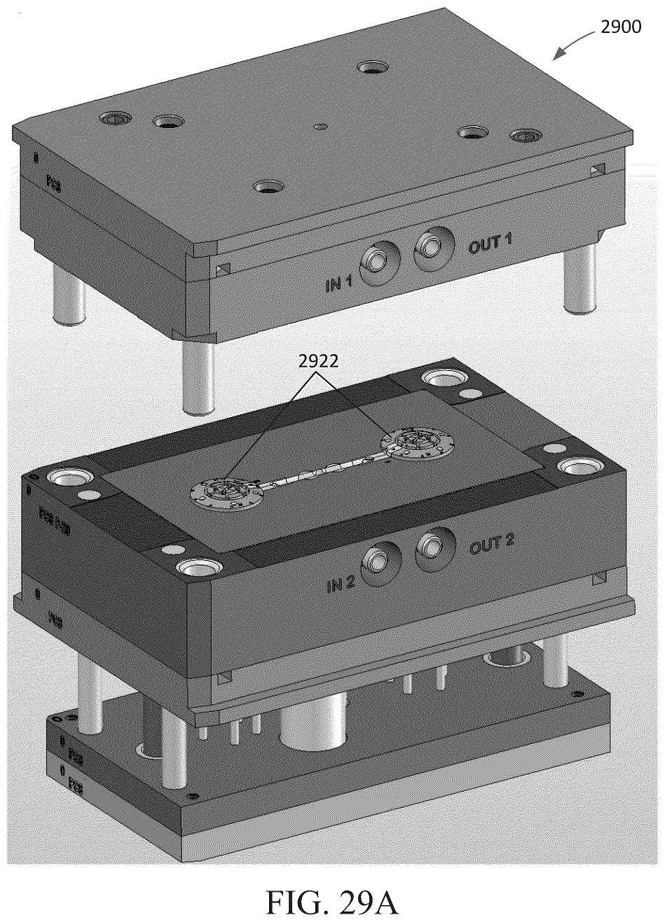

FIGS. 28A-28E illustrate various views of a mold assembly 2800 for detector chamber covers. The mold assembly 2800 includes two halves that mate together to form molds 2822. The molds 2822 are openings between the two halves of the molds assembly 2800 into which material can be placed (e.g., injected) and assume the shape of the molds 2822. Material can be inserted into the molds 2822 via channel 2871. Material can be injected into molds in a viscous/liquid state such as when heated and can then harden into a more solid state as it cools within the molds 2822 and thus take the shape of the molds 2822. Various materials can be injected into the molds 2822 including glass, plastic, or any of the other materials shown and/or described herein. In this example, mold assembly 2800 includes two molds 2822 for creating two sets of detector chamber covers 2880. Once the sets of detector chamber covers 2880 have sufficiently hardened after having been injected molded, the sets of detector chamber covers 2880 can be removed from the mold assembly 2800. In this example, each set of detector chamber covers 2880 includes six detector chamber covers that are integrally connected and formed from the same material at the same time during the same injection molding process.

FIGS. 29A-29E illustrate various views of a mold assembly 2900 for frames (also referred to as light barrier constructs). The mold assembly 2900 includes two halves that mate together to form molds 2922. The molds 2922 are openings between the two halves of the molds assembly 2900 into which material can be placed (e.g., injected) and assume the shape of the molds 2922. Material can be inserted into the molds 2922 via channel 2971. Material can be injected into molds in a viscous/liquid state such as when heated and can then harden into a more solid state as it cools within the molds 2922 and thus take the shape of the molds 2922. Various materials can be injected into the molds 2922 including glass, plastic, or any of the other materials shown and/or described herein. In this example, mold assembly 2900 includes two molds 2922 for creating two frames 2980. The frames 2980 can be injected molded over emitter chamber covers 2780 and detector chamber covers 2880. For example, the emitter chamber covers 2780 and detector chamber covers 2880 (after having already been formed themselves) can be placed on the molds 2922 and material can then be overmolded on the emitter chamber covers 2780 and detector chamber covers 2880 to form the frame 2980. Once the frames 2980 have sufficiently hardened after having been injected molded, the frames 2980 can be removed from the mold assembly 2900 (together with the emitter chamber covers 2780 and detector chamber covers 2880). The overmolded assembly, including the frame 2980, emitter chamber covers 2780, and detector chamber covers 2880 can then undergo post processing and/or fusing to bond the frame 2980, emitter chamber covers 2780, and detector chamber covers 2880 into a single unitary structure.

FIGS. 29D-29E illustrate close-up views of mold 2922. Properly orienting emitter chamber covers 2780 and detector chamber covers 2880 within frame 2980 may be important. For example, emitter chamber covers 2780 and detector chamber covers 2880 may need to be oriented relative to each other and/or relative to the frame 2980 within certain tolerances to ensure consistency and quality of the final molded assembly. Mold 2922 includes one or more receptacles 2937 that can receive bridging portions of emitter chamber covers 2780. Receptacles 2937 can be sized and shaped to correspond to the size and shape of bridging portions of emitter chamber covers 2780. For example, receptacles 2937 may be tapered to facilitate introducing the bridging portions into the receptacles 2937. For example, receptacles 2937 can reorient the emitter chamber covers 2780, if necessary, into the proper position as the emitter chamber covers 2780 are being placed on the mold 2922. Receptacles 2937 can hold the emitter chamber covers 2780 in place to inhibit them from moving when the emitter chamber covers 2780 are being overmolded with frame 2980.

Mold 2922 includes one or more protrusions 2939 that can mate with indentations of detector chamber covers 2880. Protrusions 2939 can be sized and shaped to correspond to the size and shape of indentations of detector chamber covers 2880. For example, protrusions 2939 may be tapered to facilitate mating the protrusions 2939 with indentations of detector chamber covers 2880. For example, protrusions 2939 can reorient the detector chamber covers 2880, if necessary, into the proper position as the detector chamber covers 2880 are being placed on the mold 2922. Protrusions 2939 can hold the detector chamber covers 2880 in place to inhibit them from moving when the detector chamber covers 2880 are being overmolded with frame 2980.

### Additional Considerations

Certain categories of persons, such as caregivers, clinicians, doctors, nurses, and friends and family of a user, may be used interchangeably to describe a person providing care to the user. Furthermore, patients or users used herein interchangeably refer to a person who is wearing a sensor or is connected to a sensor or whose measurements are used to determine a physiological parameter or a condition. Parameters may be, be associated with, and/or be represented by, measured values, display icons, alphanumeric characters, graphs, gages, power bars, trends, or combinations. Real time data may correspond to active monitoring of a user, however, such real time data may not be synchronous to an actual physiological state at a particular moment. Measurement value(s) of a parameter and the parameter used herein such as, SpO2, RR, PaO2 and the like, unless specifically stated otherwise, or otherwise understood with the context as used is generally intended to convey a measurement or determination that is responsive to the physiological parameter.

Although certain implementations and examples have been described herein, it will be understood by those skilled in the art that many aspects of the systems and devices shown and described in the present disclosure may be differently combined and/or modified to form still further implementations or acceptable examples. All such modifications and variations are intended to be included herein within the scope of this disclosure. A wide variety of designs and approaches are possible. No feature, structure, or step disclosed herein is essential or indispensable. The various features and processes described herein may be used independently of one another, or may be combined in various ways. For example, elements may be added to, removed from, or rearranged compared to the disclosed example implementations. All possible combinations and sub-combinations are intended to fall within the scope of this disclosure.

Any methods and processes described herein are not limited to any particular sequence, and the blocks or states relating thereto can be performed in other sequences that are appropriate. For example, described blocks or states may be performed in an order other than that specifically disclosed, or multiple blocks or states may be combined in a single block or state, or certain method or process blocks may be omitted, or certain blocks or states may be performed in a reverse order from what is shown and/or described. The example blocks or states may be performed in serial, in parallel, or in some other manner. Blocks or states may be added to or removed from the disclosed example implementations.

The methods disclosed herein may include certain actions taken by a practitioner; however, they can also include any third-party instruction of those actions, either expressly or by implication.

The methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions stored in a memory or other non-transitory computer-readable storage medium or device (e.g., solid state storage devices, disk drives, etc.). The various functions disclosed herein may be embodied in such program instructions, and/or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. The computer system may be a cloud-based computing system whose processing resources are shared by multiple distinct entities or other users. The systems may also be transmitted as generated data signals (for example, as part of a carrier wave or other analog or digital propagated signal) on a variety of computer-readable transmission mediums, including wireless-based and wired/cable-based mediums, and may take a variety of forms (for example, as part of a single or multiplexed analog signal, or as multiple discrete digital packets or frames).

Many other variations than those described herein will be apparent from this disclosure. For example, depending on the implementation, certain acts, events, or functions of any of the algorithms described herein can be performed in a different sequence, can be added, merged, or left out altogether (for example, not all described acts or events are necessary for the practice of the algorithms). Moreover, in certain implementations, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

Various illustrative logical blocks, processes, routines, and algorithm steps that may be described in connection with the disclosure herein can be implemented as electronic hardware (e.g., ASICs or FPGA devices), computer software that runs on computer hardware, or combinations of both. Various illustrative components, blocks, and steps may be described herein generally in terms of their functionality. Whether such functionality is implemented as specialized hardware versus software running on general-purpose hardware depends upon the particular application and design constraints imposed on the overall system. The described functionality can be implemented in varying ways for each particular application, but such implementation decisions should not be interpreted as causing a departure from the scope of the disclosure.

Moreover, various illustrative logical blocks and processes that may be described in connection with the implementations disclosed herein can be implemented or performed by a machine, such as a general purpose processor, a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. A general purpose processor can be a microprocessor, controller, microcontroller, or state machine, combinations of the same, or the like. A processor can include electrical circuitry configured to process computer-executable instructions. A processor can include an FPGA or other programmable devices that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. Although described herein primarily with respect to digital technology, a processor may also include primarily analog components. For example, some, or all, of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of any method, process, routine, or algorithm described in connection with the disclosure herein can be embodied directly in hardware, in a software program executed by a processor, or in a combination of the two. A software program can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain features, elements, and/or steps are optional. Thus, such conditional language is not generally intended to imply that features, elements, and/or steps are in any way required or that one or more embodiments necessarily include logic for deciding, with or without other input or prompting, whether these features, elements, and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 10 degrees, 5 degrees, 3 degrees, or 1 degree. As another example, in certain embodiments, the terms “generally perpendicular” and “substantially perpendicular” refer to a value, amount, or characteristic that departs from exactly perpendicular by less than or equal to 10 degrees, 5 degrees, 3 degrees, or 1 degree.

As used herein, “real-time” or “substantial real-time” may refer to events (e.g., receiving, processing, transmitting, displaying etc.) that occur at a same time as each other, during a same time as each other, or overlap in time with each other. “Real-time” may refer to events that occur at distinct or non-overlapping times the difference between which is imperceptible and/or inconsequential to humans such as delays arising from electrical conduction or transmission. A human may perceive real-time events as occurring simultaneously, regardless of whether the real-time events occur at an exact same time. As a non-limiting example, “real-time” may refer to events that occur within a time frame of each other that is on the order of milliseconds, seconds, tens of seconds, or minutes. For example, “real-time” may refer to events that occur within a time frame of less than 1 minute, less than 30 seconds, less than 10 seconds, less than 1 second, less than 0.05 seconds, less than 0.01 seconds, less than 0.005 seconds, less than 0.001 seconds, etc.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

As used herein, “system,” “instrument,” “apparatus,” and “device” generally encompass both the hardware (for example, mechanical and electronic) and, in some implementations, associated software (for example, specialized computer programs for operational control) components.

It should be emphasized that many variations and modifications may be made to the herein-described implementations, the elements of which are to be understood as being among other acceptable examples. All such modifications and variations are intended to be included herein within the scope of this disclosure. Any section headings used herein are merely provided to enhance readability and are not intended to limit the scope of the implementations disclosed in a particular section to the features or elements disclosed in that section. The foregoing description details certain implementations. It will be appreciated, however, that no matter how detailed the foregoing appears in text, the systems and methods can be practiced in many ways. As is also stated herein, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

Those of skill in the art would understand that information, messages, and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

What is claimed is:

1. A wearable health monitoring device configured to secure to a wrist area of a wearer, the device comprising:
a housing including a front side and a back side, the back side configured to face tissue of the wearer when the device is worn; and
a sensor assembly positioned on the back side of the housing and comprising:
emitters positioned on a surface of a substrate;
one or more detectors positioned on the surface of the substrate;
a frame positioned adjacent to the surface of the substrate and configured to inhibit transmission of optical radiation through the frame, the frame comprising:
an emitter chamber housing the emitters; and
one or more detector chambers housing the one or more detectors; and
a substance positioned on the surface of the substrate between the emitters and the one or more detectors, wherein the substance is configured to contact the frame between the emitter chamber and the one or more detector chambers, wherein the substance is configured to inhibit the transmission of optical radiation through the substance between the frame and the surface of the substrate from the emitter chamber to the one or more detector chambers.

2. The wearable health monitoring device of claim 1, wherein the substance forms at least one closed loop on the surface of the substrate.

3. The wearable health monitoring device of claim 1, wherein the substance surrounds the emitters on the substrate.

4. The wearable health monitoring device of claim 1, wherein the substance surrounds the one or more detectors on the substrate.

5. The wearable health monitoring device of claim 1, wherein the substance is opaque.

6. The wearable health monitoring device of claim 1, wherein the substance is adhesive and is configured to adhere to the frame and to the surface of the substrate.

7. The wearable health monitoring device of claim 1, wherein the substance is viscous when applied to the surface of the substrate and solidifies after applied to the surface of the substrate.

8. The wearable health monitoring device of claim 1, wherein the substance comprises a compressible foam.

9. The wearable health monitoring device of claim 1, wherein the one or more detector chambers surround the emitter chamber.

10. The wearable health monitoring device of claim 1, wherein each of the one or more detector chambers houses one or more of the one or more detectors.

11. The wearable health monitoring device of claim 1, wherein the frame is opaque and contacts the tissue of the wearer when the device is worn to inhibit transmission of optical radiation along the tissue of the wearer between the emitter chamber and the one or more detector chambers or between the one or more detector chambers.

12. The wearable health monitoring device of claim 1, wherein the emitters form a first group of emitters, wherein the sensor assembly comprises a second group of emitters, wherein the frame comprises a second emitter chamber housing the second group of emitters, wherein a portion of the substance is positioned on the surface of the substrate between the first group of emitters and the second group of emitters, wherein the substance is configured to contact the frame between the emitter chamber and the second emitter chamber, wherein the substance is configured to inhibit the transmission of optical radiation through the substance between the frame and the surface of the substrate from the emitter chamber to the second emitter chamber.

13. The wearable health monitoring device of claim 12, wherein the substance forms a first closed loop around the emitter chamber and a second closed loop around the second emitter chamber.

14. A wearable health monitoring device configured to secure to a wrist area of a wearer, the device comprising:
a housing including a front side and a back side, the back side configured to face tissue of the wearer when the device is worn; and
a sensor assembly positioned on the back side of the housing and comprising:
a first group of emitters positioned on a surface of a substrate;
a second group of emitters positioned on the surface of the substrate;
one or more detectors positioned on the surface of the substrate;
a frame positioned adjacent to the surface of the substrate and configured to inhibit transmission of optical radiation through the frame, the frame comprising:
a first emitter chamber housing the first group of emitters;
a second emitter chamber housing the second group of emitters; and
one or more detector chambers housing the one or more detectors; and
a substance positioned on the surface of the substrate between the first group of emitters and the second group of emitters, wherein the substance is configured to contact the frame between the first emitter chamber and the second emitter chamber, wherein the substance is configured to inhibit the transmission of optical radiation through the substance between the frame and the surface of the substrate from the first emitter chamber to the second emitter chamber.

15. The wearable health monitoring device of claim 14, wherein the substance is opaque.

16. The wearable health monitoring device of claim 14, wherein the substance is adhesive and is configured to adhere to the frame and to the surface of the substrate.

17. The wearable health monitoring device of claim 14, wherein the frame is opaque and contacts the tissue of the wearer when the device is worn to inhibit transmission of optical radiation along the tissue of the wearer between the first and second emitter chambers, between the first and second emitter chambers and the one or more detector chambers, or between the one or more detector chambers.

18. A wearable health monitoring device configured to secure to a wrist area of a wearer, the device comprising:
a housing including a front side and a back side, the back side configured to face tissue of the wearer when the device is worn; and
a sensor assembly positioned by the back side of the housing and comprising:
emitters positioned on a surface of a substrate;
one or more detectors positioned on the surface of the substrate; and
a frame positioned adjacent to the surface of the substrate and configured to inhibit transmission of optical radiation through the frame, the frame comprising:
an emitter chamber housing the emitters and forming an emitter chamber window for optical radiation to pass through; and
one or more detector chambers housing the one or more detectors and forming one or more detector chamber windows for optical radiation to pass through,
wherein the frame is opaque and configured to directly contact the tissue of the wearer when the device is worn to inhibit transmission of optical radiation between the frame and the tissue of the wearer.

19. The wearable health monitoring device of claim 18, wherein the sensor assembly does not comprise a light transmissive material positioned between the frame and the tissue of the wearer.

20. The wearable health monitoring device of claim 18, wherein the frame comprises one or more edges defining the emitter chamber window and the one or more detector chamber windows, wherein the one or more edges of the frame are configured to contact the tissue of the wearer when the device is worn.
